(19) United States
(12) Patent Application Publication
(10) Pub. No.: US 2015/0148432 A1
(43) Pub. Date: May 28, 2015

(54) ASSESSING SUSCEPTIBILITY TO VASCULAR DISORDERS

(71) Applicant: University of Iowa Research Foundation, Iowa City, IA (US)

(72) Inventor: Gregory S. Hageman, Salt Lake City, UT (US)

(21) Appl. No.: 14/331,173

(22) Filed: Jul. 14, 2014

Related U.S. Application Data
(63) Continuation of application No. 12/740,962, filed on Aug. 20, 2010, now abandoned.
(60) Provisional application No. 60/984,702, filed on Nov. 1, 2007.

(51) Int. Cl. C12Q 1/68

(52) U.S. Cl. CPC C12Q 1/6883; C12Q 2600/156
USPC 514/789; 435/6.11; 506/16; 506/9

(57) ABSTRACT

The invention provides methods and reagents for determination of risk and treatment of a vascular disorder such as abdominal aortic aneurysm (AAA) by detecting presence of gene polymorphisms and/or genetic profiles associated with an elevated or a reduced risk of the disorder. In an embodiment, the present invention provides methods and reagents for determining sequence variants in the genome of an individual which facilitate assessment of risk for developing such diseases.

### RELATED APPLICATIONS

This application claims the benefit of the priority date of U.S. provisional application No. 60/984,702, which was filed on Nov. 1, 2007, the contents of which are incorporated herein by reference in their entirety.

### FEDERALLY SPONSORED RESEARCH AND DEVELOPMENT

This invention was made with government support under NIH ROI EY11515 and R24 EY017404, awarded by the National Institutes of Health. The government has certain rights in the invention.

### FIELD OF THE INVENTION

The invention relates to risk determination, diagnosis and prognosis of vascular disorders such as abdominal aortic aneurysm (AAA).

### BACKGROUND OF THE INVENTION

Pathological changes associated with many disorders or conditions are reflected in the protein profile of serum and plasma (because blood comes into contact with most of the tissues in the human body), as well as other body fluid, such as urine. Monitoring the levels (and changes in levels) of such proteins, or “biomarkers” is useful for diagnosis and prognosis of diseases, disorders or conditions. In addition, changes in levels of biomarker can serve as surrogate endpoints for assessing the effects and efficacy of therapeutic interventions.

An aortic aneurysm is a vascular disorder involving swelling or expansion of the aorta resulting from weakness in the aortic wall. Although stretching of the aorta can cause physical discomfort, the serious medical risk is rupture of the aorta, which causes severe pain, internal bleeding and, absent prompt treatment, death. Aneuryms are also a source of blood clots, which can cause many complications, including a heart attack or stroke. The most common aneurysm is abdominal aortic aneurysm (AAA), which occurs in the abdominal aorta that supplies blood to the abdomen, pelvis and legs.

AAA develops slowly over time and is most common in older individuals, with the average age at diagnosis being 65-70 years. Risk factors for AAA include high blood pressure, smoking, cholesterol and obesity. AAA is currently diagnosed by abdominal ultrasound, abdominal CT scanning and aortic angiography. Very little is known about the genetic basis of the disease. Therapeutic options are available for individuals with AAA, including surgical replacement of the abdominal vessel and endovascular stent grafting, and others are being developed. Some patients are afflicted with both AAA and age-related macular degeneration (AMD).

Age-related macular degeneration (AMD) is the leading cause of irreversible vision loss in the developed world, affecting approximately 15% of individuals over the age of 60. The prevalence of AMD increases with age: mild, or early, forms occur in nearly 30%, and advanced forms in about 7%, of the population that is 75 years and older. Clinically, AMD is characterized by a progressive loss of central vision attributable to degenerative changes that occur in the macula, a specialized region of the neural retina and underlying tissues. In the most severe, or exudative, form of the disease neovascular fronds derived from the choroidal vasculature breach Bruch's membrane and the retinal pigment epithelium (RPE) typically leading to detachment and subsequent degeneration of the retina.

Biomarkers for AAA (including AAA in combination with age-related macular degeneration) have been described in US 2008-0118928, and US 2008-0152659, incorporated by reference in their entirety. However, new and methods are needed to assessment of a patient's risk of developing vascular disorders such as AAA and predicting the course of development of the condition. The present invention provides these and other benefits.

### SUMMARY OF THE INVENTION

The invention arises, in part, from a high density, large sample size, genetic association study designed to detect genetic characteristics associated with vascular disorders such as as abdominal aortic aneurysm (AAA), cerebral hemorrhage, and other conditions. The study revealed a large number of new SNPs never before reported and a still larger number of SNPs (and/or combination of certain SNPs) which were not previously reported to be associated with risk for, or protection from, the disease. The invention disclosed herein thus relates to the discovery of genetic polymorphisms that are associated with increased or decreased risk of abdominal aortic aneurysm (AAA). The polymorphisms are found in or near genes such as CR1, C1RL and SDC4. The informative value of many of the specific SNPs disclosed herein has never before been recognized or reported, as far as the inventor is aware. The invention provides methods of screening for individuals at risk of having or developing AAA and/or for predicting the likely progression of early- or mid-stage established disease and/or for predicting the likely outcome of a particular therapeutic or prophylactic strategy.

In one aspect, the invention provides a diagnostic method of determining an individual's risk or propensity for AAA or an AAA-associated vascular disorder, or for predicting the course of progression of AAA, comprising screening (directly or indirectly) for the presence or absence of a genetic profile that includes one or more, typically multiple, single nucleotide polymorphisms selected from Tables 1A and 2A, which are informative of an individual's (increased or decreased) risk for developing AAA. For example, the invention provides a method of determining an individual's risk of AAA or an AAA-associated vascular disorder comprising screening the genome of the individual for the presence or absence of a genetic profile characterized by at least one polymorphism selected from Table 1A and/or Table 2A associated with increased risk for or protection against AAA, wherein the presence of a said genetic profile is considered to be indicative of the individual's relative risk of AAA.

A subset of individuals with AAA have been found to also suffer from AMD. Accordingly, the invention also provides a diagnostic method of determining an individual's risk or propensity for AAA, or for predicting the course of progression, of AAA in combination with AMD (“AAA+AMD”), comprising screening (directly or indirectly) for the presence or absence of a genetic profile that includes one or more, or multiple, single nucleotide polymorphisms selected from Table 2A, which are informative of an individual's (increased or decreased) risk for developing AAA+AMD. Optionally the individual is known or suspected to have, or has at least one symptom of AAA or AMD. Optionally, the individual has been found to have a genetic profile that indicates an increased risk of AAA or AMD. For example, the individual has at least one predisposing polymorphism for AMD or AAA.

In one embodiment, the polymorphisms include at least 1, at least 2, at least 5, or at least 10 single nucleotide polymorphisms selected from the Tables.

In one embodiment, a method for determining an individual's risk or propensity of AAA, or for predicting the course of progression of AAA includes screening for a combination of at least one, typically multiple, predisposing polymorphism and at least one, typically multiple, protective polymorphism set forth in Tables 1A and 2A.

Risk polymorphisms indicate that an individual has increased risk of having, or increased susceptibility to development or progression of a disease or disorder relative to the control population. Protective polymorphisms indicate that the individual has a reduced likelihood of development or progression of a disease or disorder relative to the control population. Neutral polymorphisms do not segregate significantly with risk or protection, and have limited or no diagnostic or prognostic value. Additional, previously known informative polymorphisms can and typically will be included in the screen.

In another embodiment, a method for determining an individual's risk or propensity of AAA or for predicting the course of progression of AAA+AMD includes screening for a combination of at least one, typically multiple, predisposing polymorphism and at least one, typically multiple, protective polymorphism set forth in Table 2A.

In another embodiment, a method for determining an individual's risk or propensity for AAA or for predicting the course of progression of AAA includes screening additionally for deletions within the RCA locus that are associated with AAA risk. An exemplary deletion that is indicative of risk is a deletion at least portions of the FHR3 and FHR1 genes. See, e.g., Hageman et al., 2006, “Extended haplotypes in the complement factor H (CFH) and CFH-related (CFHR) family of genes protect against age-related macular degeneration: characterization, ethnic distribution and evolutionary implications ,”Ann Med. 38:592-604, U.S. Patent Application Publication No. US 2008/152659, and International Pub. No. WO 2008/008986, all of which are incorporated by reference in their entirety.

The methods can include inspecting a data set indicative of genetic characteristics previously derived from analysis of the individual's genome. A data set of genetic characteristics of the individual can include, for example, a listing of single nucleotide polymorphisms in the individual's genome or a complete or partial sequence of the individual's genomic DNA. Alternatively, the methods include obtaining and analyzing a nucleic acid sample (e.g., DNA or RNA) from an individual to determine whether the DNA contains informative polymorphisms, such as by combining a nucleic acid sample from the subject with one or more polynucleotide probes capable of hybridizing selectively to a nucleic acid carrying the polymorphism. In another embodiment, the methods include obtaining a biological sample from the individual and analyzing the sample from the individual to determine whether the individual's proteome contains an allelic variant protein isoform that is a consequence of the presence of a polymorphism in the individual's genome.

In another aspect, the invention provides a method of treating, preventing, or delaying development of symptoms of AAA in an individual (e.g., an individual in whom a genetic profile indicative of elevated risk of developing AAA is detected), comprising prophylactically or therapeutically treating an individual identified as having a genetic profile including one or more single nucleotide polymorphisms (SNPs) selected from Tables 1A and 2A. Optionally, the individual is at increased risk for a combination of AAA and AMD (AAA+AMD).

In yet another aspect, the invention provides a method of treating, preventing, or delaying development of symptoms of AAA and/or AMD in an individual (e.g., an individual in whom a genetic profile indicative of elevated risk for both AAA and AMD is detected), comprising prophylactically or therapeutically treating an individual identified as having a genetic profile including one or more single nucleotide polymorphisms (SNPs) selected from Table 2A. For example, the invention includes a method for therapeutically treating AAA (or prophylactically treating the onset or progression of AAA), the method comprising (i) identifying an individual as having a genetic profile characterized by polymorphisms indicative of risk for developing AAA, wherein the genetic profile comprises at least one polymorphism selected from Table 1A or Table 2A, and (ii) therapeutically or prophylactically treating the individual. Optionally the individual has at least one symptom of AMD, or is believed to have or suspected to be at risk for AAA or AMD. For example, the individual has at least one predisposing polymorphism for AMD or AAA.

In another aspect, the invention provides detectably labeled oligonucleotide probes or primers for hybridization with DNA sequence in the vicinity of at least one polymorphism to facilitate identification of the base present in the individual's genome. In one embodiment, a set of oligonucleotide primers hybridizes adjacent to at least one polymorphism disclosed herein for inducing amplification thereof, thereby facilitating sequencing of the region and determination of the base present in the individual's genome at the sites of the polymorphism. Preferred polymorphisms for detection include the polymorphisms listed in Table 1A and Table 2A. Further, one of skill in the art will appreciate that other methods for detecting polymorphisms are well known in the art.

In another aspect, the invention relates to a healthcare method that includes authorizing the administration of, or authorizing payment for the administration of, an assay to determine an individual's risk of having AAA, or an individual's susceptibility for development or progression of AAA. The method includes screening for the presence or absence of a genetic profile that includes one or more SNPs selected from Tables 1A and 2A (for AAA) or Table 2A (for AAA+AMD).

### DETAILED DESCRIPTION OF THE INVENTION

### I. Definitions and Conventions

The term “polymorphism” refers to the occurrence of two or more genetically determined alternative sequences or alleles in a population. Each divergent sequence is termed an allele, and can be part of a gene or located within an intergenic or non-genic sequence. A diallelic polymorphism has two alleles, and a triallelic polymorphism has three alleles. Diploid organisms can contain two alleles and can be homozygous or heterozygous for allelic forms. The first identified allelic form is arbitrarily designated the reference form or allele; other allelic forms are designated as alternative or variant alleles. The most frequently occurring allelic form in a selected population is typically referred to as the wild-type form.

A “polymorphic site” is the position or locus at which sequence divergence occurs at the nucleic acid level and is sometimes reflected at the amino acid level. The polymorphic region or polymorphic site refers to a region of the nucleic acid where the nucleotide difference that distinguishes the variants occurs, or, for amino acid sequences, a region of the amino acid sequence where the amino acid difference that distinguishes the protein variants occurs. A polymorphic site can be as small as one base pair, often termed a “single nucleotide polymorphism” (SNP). The SNPs can be any SNPs in loci identified herein, including intragenic SNPs in exons, introns, or upstream or downstream regions of a gene, as well as SNPs that are located outside of gene sequences. Examples of such SNPs include, but are not limited to, those provided in the Tables hereinbelow.

Individual amino acids in a sequence are represented herein as AN or NA, wherein A is the amino acid in the sequence and N is the position in the sequence. In the case that position N is polymorphic, it is convenient to designate the more frequent variant as A₁N and the less frequent variant as NA₂. Alternatively, the polymorphic site, N, is represented as A₁NA₂, wherein A₁ is the amino acid in the more common variant and A₂ is the amino acid in the less common variant. Either the one-letter or three-letter codes are used for designating amino acids (see Lehninger, Biochemistry 2nd ed., 1975, Worth Publishers, Inc. New York, N.Y.: pages 73-75, incorporated herein by reference). For example, 150V represents a single-amino-acid polymorphism at amino acid position 50 of a given protein, wherein isoleucine is present in the more frequent protein variant in the population and valine is present in the less frequent variant.

Similar nomenclature can be used in reference to nucleic acid sequences. In the Tables provided herein, each SNP is depicted by “N₁/N₂” where N₁ is a nucleotide present in a first allele referred to as Allele 1, and N₂ is another nucleotide present in a second allele referred to as Allele 2. It will be clear to those of skill in the art that in a double-stranded form, the complementary strand of each allele will contain the complementary base at the polymorphic position.

The term “genotype” as used herein denotes one or more polymorphisms of interest found in an individual, for example, within a gene of interest. Diploid individuals have a genotype that comprises two different sequences (heterozygous) or one sequence (homozygous) at a polymorphic site.

The term “haplotype” refers to a DNA sequence comprising one or more polymorphisms of interest contained on a subregion of a single chromosome of an individual. A haplotype can refer to a set of polymorphisms in a single gene, an intergenic sequence, or in larger sequences including both gene and intergenic sequences, e.g., a collection of genes, or of genes and intergenic sequences. For example, a haplotype can refer to a set of polymorphisms on chromosome 12 within or near the C1RL gene, or on chromosome 20 within or near the SDC4 gene, or on chromosome 1 within or near the CR1 gene, e.g. within the genes and/or within intergenic sequences (i.e., intervening intergenic sequences, upstream sequences, and downstream sequences that are in linkage disequilibrium with polymorphisms in the genic region). The term “haplotype” can refer to a set of single nucleotide polymorphisms (SNPs) found to be statistically associated on a single chromosome. A haplotype can also refer to a combination of polymorphisms (e.g., SNPs) and other genetic markers (e.g., a deletion) found to be statistically associated on a single chromosome. A haplotype, for instance, can also be a set of maternally inherited alleles, or a set of paternally inherited alleles, at any locus.

The term “genetic profile,” as used herein, refers to a collection of one or more single nucleotide polymorphisms including a polymorphism shown in Tables 1A and 2A optionally in combination with other genetic characteristics such as deletions, additions or duplications, and optionally combined with other SNPs associated with AAA risk or protection, including but not limited to those in Tables 1A and 2A. The polymorphisms in both Tables 1A and 2A are associated with risk of AAA. In some cases the subject at risk of AAA develops or is at increased risk of having or developing AMD. The polymorphisms in Table 2A are associated with risk of both AAA+AMD. Thus, a genetic profile, as the phrase is used herein, is not limited to a set of characteristics defining a haplotype, and can include SNPs from diverse regions of the genome. For example, a genetic profile for AAA includes one or a subset of single nucleotide polymorphisms selected from Tables 1A and 2A, optionally in combination with other genetic characteristics associated with AAA. Also for example, a genetic profile for AAA+AMD includes one or a subset of single nucleotide polymorphisms selected from Table 2A, optionally in combination with other genetic characteristics associated with AAA. It is understood that while one SNP in a genetic profile can be informative of an individual's increased or decreased risk (i.e., an individual's propensity or susceptibility) to have or develop a vascular disorder such as AAA, more than one SNP in a genetic profile can and typically will be analyzed and will be more informative of an individual's increased or decreased risk of having or developing a vascular disorder. A genetic profile can include at least one SNP disclosed herein in combination with other polymorphisms or genetic markers (e.g., a deletion) and/or clinical data known to be associated with AAA or AMD. Risk factors for AAA include atherosclerosis, high blood pressure, smoking, high cholesterol, obesity, emphysema, genetic factors including family history, and the male gender. AAA is most frequently seen in males over 60 with one or more risk factors. In some cases, a SNP can reflect a change in regulatory or protein coding sequences that change gene product levels or activity in a manner that results in increased likelihood of development of disease. In addition, it will be understood by a person of skill in the art that one or more SNPs that are part of a genetic profile maybe in linkage disequilibrium with, and serve as a proxy or surrogate marker for, another genetic marker or polymorphism that is causative, protective, or otherwise informative of disease.

The term “gene,” as used herein, refers to a region of a DNA sequence that encodes a polypeptide or protein, intronic sequences, promoter regions, and upstream (i.e., proximal) and downstream (i.e., distal) non-coding transcription control regions (e.g., enhancer and/or repressor regions).

The term “allele,” as used herein, refers to a sequence variant of a genetic sequence (e.g., typically a gene sequence as described hereinabove, optionally a protein coding sequence). For purposes of this application, alleles can but need not be located within a gene sequence. Alleles can be identified with respect to one or more polymorphic positions such as SNPs, while the rest of the gene sequence can remain unspecified. For example, an allele can be defined by the nucleotide present at a single SNP, or by the nucleotides present at a plurality of SNPs. In certain embodiments of the invention, an allele is defined by the genotypes of at least 1, 2, 4, 8 or 16 or more SNPs, (including those provided in Tables 1A and 2A below) in a gene.

The term “linkage” refers to the tendency of genes, alleles, loci, or genetic markers to be inherited together as a result of their location on the same chromosome or as a result of other factors. Linkage can be measured by percent recombination between the two genes, alleles, loci, or genetic markers. Some linked markers can be present within the same gene or gene cluster.

In population genetics, linkage disequilibrium is the non-random association of alleles at two or more loci, not necessarily on the same chromosome. It is not the same as linkage, which describes the association of two or more loci on a chromosome with limited recombination between them. Linkage disequilibrium describes a situation in which some combinations of alleles or genetic markers occur more or less frequently in a population than would be expected from a random formation of haplotypes from alleles based on their frequencies. Non-random associations between polymorphisms at different loci are measured by the degree of linkage disequilibrium (LD). The level of linkage disequilibrium is influenced by a number of factors including genetic linkage, the rate of recombination, the rate of mutation, random drift, non-random mating, and population structure “Linkage disequilibrium” or “allelic association” thus means the preferential association of a particular allele or genetic marker with another specific allele or genetic marker more frequently than expected by chance for any particular allele frequency in the population. A marker in linkage disequilibrium with an informative marker can be useful in detecting susceptibility to disease even if the informative marker does not contribute (or there is no apparent theory as to how it could contribute) to the cause of the disease. A SNP that is in linkage disequilibrium with a causative, protective, or otherwise informative SNP or genetic marker is referred to as a “proxy” or “surrogate” SNP. A proxy SNP can be in at least 50%, 60%, or 70% in linkage disequilibrium with the causative SNP, and preferably is at least about 80%, 90%, and most preferably 95%, or about 100% in LD with the genetic marker.

A “causative” SNP is a SNP having an allele that is directly responsible for a difference in risk of having or developing a disorder or progression of the disorder. Generally, a causative SNP has an allele producing an alteration in gene expression or in the expression, structure, and/or function of a gene product, and therefore is most predictive of a possible clinical phenotype. One such class includes SNPs falling within regions of genes encoding a polypeptide product, i.e. “coding SNPs” (cSNPs). These SNPs can result in an alteration of the amino acid sequence of the polypeptide product (i.e., non-synonymous codon changes) and give rise to the expression of a defective or other variant protein. Furthermore, in the case of nonsense mutations, a SNP can lead to premature termination of a polypeptide product. Such variant products can result in a pathological condition, e.g., genetic disease. Examples of genes in which a SNP within a coding sequence causes a genetic disease include sickle cell anemia and cystic fibrosis.

Causative SNPs do not necessarily have to occur in coding regions; causative SNPs can occur in, for example, any genetic region that can ultimately affect the expression, structure, and/or activity of the protein encoded by a nucleic acid. Such genetic regions include, for example, those involved in transcription, such as SNPs in transcription factor binding domains, SNPs in promoter regions, in areas involved in transcript processing, such as SNPs at intron-exon boundaries that can cause defective splicing, or SNPs in mRNA processing signal sequences such as polyadenylation signal regions. Some SNPs that are not causative SNPs nevertheless are in close association with, and therefore segregate with, a disease-causing sequence. In this situation, the presence of a SNP correlates with the likely presence of, or predisposition to, or an increased risk in developing the disease. These SNPs, although not causative, are nonetheless also useful for diagnostics, disease predisposition screening, and other uses.

An “informative” or “risk-informative” SNP refers to any SNP whose sequence in an individual provides information about that individual's relative risk of having or developing AAA or relative risk of progression of AAA. An informative SNP need not be causative. Indeed, many informative SNPs have no apparent effect on any gene product, but are in linkage disequilibrium with a causative SNP. In such cases, as a general matter, the SNP is increasingly informative when it is more tightly in linkage disequilibrium with a causative SNP. For various informative SNPs, the relative risk of development or progression of AAAis indicated by the presence or absence of a particular allele and/or by the presence or absence of a particular diploid genotype.

A “nucleic acid,” “polynucleotide,” or “oligonucleotide” is a polymeric form of nucleotides of any length, can be DNA or RNA, and can be single- or double-stranded. The polymer can include, without limitation, natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). Nucleic acids and oligonucleotides can also include other polymers of bases having a modified backbone, such as a locked nucleic acid (LNA), a peptide nucleic acid (PNA), a threose nucleic acid (TNA) and any other polymers capable of serving as a template for an amplification reaction using an amplification technique, for example, a polymerase chain reaction, a ligase chain reaction, or non-enzymatic template-directed replication.

Oligonucleotides are usually prepared by synthetic means. Nucleic acids include segments of DNA, or their complements spanning any one of the polymorphic sites shown in the Tables provided herein. Except where otherwise clear from context, reference to one strand of a nucleic acid also refers to its complement strand. The segments are usually between 5 and 100 contiguous bases, and often range from a lower limit of 5, 10, 12, 15, 20, or 25 nucleotides to an upper limit of 10, 15, 20, 25, 30, 50 or 100 nucleotides (where the upper limit is greater than the lower limit). Nucleic acids between 5-10, 5-20, 10-20, 12-30, 15-30, 10-50, 20-50 or 20-100 bases are common. The polymorphic site can occur within any position of the segment. The segments can be from any of the allelic forms of DNA shown in the Tables provided herein.

“Hybridization probes” are nucleic acids capable of binding in a base-specific manner to a complementary strand of nucleic acid. Such probes include nucleic acids and peptide nucleic acids. Hybridization is usually performed under stringent conditions which are known in the art. A hybridization probe can include a “primer.”

The term “primer” refers to a single-stranded oligonucleotide capable of acting as a point of initiation of template-directed DNA synthesis under appropriate conditions, in an appropriate buffer and at a suitable temperature. The appropriate length of a primer depends on the intended use of the primer, but typically ranges from 15 to 30 nucleotides. A primer sequence need not be exactly complementary to a template, but must be sufficiently complementary to hybridize with a template. The term “primer site” refers to the area of the target DNA to which a primer hybridizes. The term “primer pair” means a set of primers including a 5′ upstream primer, which hybridizes to the 5′ end of the DNA sequence to be amplified and a 3′ downstream primer, which hybridizes to the complement of the 3′ end of the sequence to be amplified.

The nucleic acids, including any primers, probes and/or oligonucleotides can be synthesized using a variety of techniques currently available, such as by chemical or biochemical synthesis, and by in vitro or in vivo expression from recombinant nucleic acid molecules, e.g., bacterial or retroviral vectors. For example, DNA can be synthesized using conventional nucleotide phosphoramidite chemistry and the instruments available from Applied Biosystems, Inc. (Foster City, Calif); DuPont (Wilmington, Del.); or Milligen (Bedford, Mass.). When desired, the nucleic acids can be labeled using methodologies well known in the art such as described in U.S. Pat. Nos. 5,464,746; 5,424,414; and 4,948,882 all of which are herein incorporated by reference. In addition, the nucleic acids can comprise uncommon and/or modified nucleotide residues or non-nucleotide residues, such as those known in the art.

An “isolated” nucleic acid molecule, as used herein, is one that is separated from nucleotide sequences which flank the nucleic acid molecule in nature and/or has been completely or partially purified from other biological material (e.g., protein) normally associated with the nucleic acid. For instance, recombinant DNA molecules in heterologous organisms, as well as partially or substantially purified DNA molecules in solution, are “isolated” for present purposes.

The term “target region” refers to a region of a nucleic acid which is to be analyzed and usually includes at least one polymorphic site.

“Stringent” as used herein refers to hybridization and wash conditions at 50° C. or higher. Other stringent hybridization conditions can also be selected. Generally, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 50° C. As other factors can significantly affect the stringency of hybridization, including, among others, base composition, length of the nucleic acid strands, the presence of organic solvents, and the extent of base mismatching, the combination of parameters is more important than the absolute measure of any one.

Generally, increased or decreased risk associated with a polymorphism or genetic profile for a disease is indicated by an increased or decreased frequency, respectively, of the disease in a population or individuals harboring the polymorphism or genetic profile, as compared to otherwise similar individuals, who are for instance matched by age, by population, and/or by presence or absence of other polymorphisms associated with risk for the same or similar diseases. The risk effect of a polymorphism can be of different magnitude in different populations. A polymorphism, haplotype, or genetic profile can be negatively associated (“protective polymorphism”) or positively associated (“predisposing polymorphism”) with a vascular disorder such as AAA. The presence of a predisposing genetic profile in an individual can indicate that the individual has an increased risk for the disease relative to an individual with a different profile. Conversely, the presence of a protective polymorphism or genetic profile in an individual can indicate that the individual has a decreased risk for the disease relative to an individual without the polymorphism or profile.

The terms “susceptibility,” “propensity,” and “risk” refer to either an increased or decreased likelihood of an individual having or developing a disorder (e.g., a condition, illness, disorder or disease) relative to a control and/or non-diseased population. In one example, the control population can be individuals in the population (e.g., matched by age, gender, race and/or ethnicity) without the disorder, or without the genotype or phenotype assayed for.

The terms “diagnose” and “diagnosis” refer to the ability to determine or identify whether an individual has an increased likelihood (e.g., a significant or high, probability) of developing or having AAA (e.g., an area of vascular expansion) or an AAA-associated vascular disorder. As used herein, diagnosis includes a method of screening an individual or a population for increased or decreased risk of a disorder. The term “prognose” or “prognosis” refers to the ability to predict the course of the disease and/or to predict the likely outcome of a particular therapeutic or prophylactic strategy. For example, some types of AAA progress extremely rapidly. The ability to identify patients at risk for development or rapid expansion allows timely prophalyctic and therapeutic intervention.

The term “screen” or “screening” as used herein has a broad meaning. It includes processes intended for diagnosing or for determining the susceptibility, propensity, risk, or risk assessment of an asymptomatic subject for having or developing a disorder later in life. Screening also includes the prognosis of a subject, i.e., when a subject has been diagnosed with a disorder, determining in advance the progress of the disorder as well as the assessment of efficacy of therapy options to treat a disorder. Screening can be done by examining a presenting individual's DNA, RNA, or in some cases, protein, to assess the presence or absence of the various SNPs disclosed herein (and typically other SNPs and genetic or behavioral characteristics) so as to determine where the individual lies on the spectrum of disease risk-neutrality-protection. Proxy SNPs can substitute for any of these SNPs. A sample such as a blood sample can be taken from the individual for purposes of conducting the genetic testing using methods known in the art or yet to be developed. Alternatively, if a health provider has access to a pre-produced data set recording all or part of the individual's genome (e.g. a listing of SNPs in the individual's genome), screening can be done simply by inspection of the database, optimally by computerized inspection. Screening can further comprise the step of producing a report identifying the individual and the identity of alleles at the site of at least one or more polymorphisms shown in Tables 1A and 2A.

### II. Introduction

A study was conducted to elucidate potential associations between vascular discorders and selected SNPs, including SNPs in or near complement system genes and other selected genes. Examples of vascular disorders associated with AAA include aneurysms, such as brain intracranial aneurysm, thoracic aortic aneurysm, popliteal artery aneurysm, or femoral artery aneurysms.

The associations discovered form the basis of the present invention, which provides methods for identifying individuals at increased risk, or at decreased risk, relative to the general population for a vascular disorder such as AAA. The invention also provides methods for identifying individuals at increased or decreased risk for both AAA and AMD. The invention also allows identification of AAA individuals who are at increased or decreased risk for AMD relative to other AAA individuals. The present invention also provides kits, reagents and devices useful for making such determinations. The methods and reagents of the invention are also useful for determining prognosis.

Use of Polymorphisms to Detect Risk and Protection

The present invention provides a method for detecting an individual's increased or decreased risk for a vascular disorder such as AAA by detecting the presence of certain polymorphisms present in the individual's genome that are informative of his or her future disease status (including prognosis and appearance of signs of disease). The presence of such a polymorphism can be regarded as indicative of an individual's risk (increased or decreased) for the disease, especially in individuals who lack other predisposing or protective polymorphisms for the same disease. Even in cases where the predictive contribution of a given polymorphism is relatively minor by itself, genotyping contributes information that nevertheless can be useful in characterizing an individual's predisposition to developing a disease. The information can be particularly useful when combined with genotype information from other loci (e.g., the presence of a certain polymorphism can be more predictive or informative when used in combination with at least one other polymorphism).

III. New SNPs Associated with Propensity for Vascular Disorders

In order to identify new single nucleotide polymorphisms (SNPs) associated with increased or decreased risk of having or developing vascular disorders such as AAA, a pool of selected genes including 74 complement pathway-associated genes (and a number of inflammation-associated genes including toll-like receptors, or TLRs) were selected for SNP discovery. New SNPs in the candidate genes were discovered from a pool of 475 DNA samples derived from study participants with a history of AAA using a multiplexed SNP enrichment technology called Mismatch Repair Detection (ParAllele Biosciences/Affymetrix), an approach that enriches for variants from pooled samples. This SNP discovery phase (also referred to herein as Phase I) was conducted using DNA derived solely from individuals with AAA (including a set of subjects with both AAA and AMD) based upon the rationale that the discovered SNPs might be highly relevant to disease (e.g., AAA-associated).

### IV. Association of SNPs and Vascular Conditions

In Phase II of the study, 1162 DNA samples were employed for genotyping known and newly discovered SNPs in 340 genes. Genes investigated in Phase II included the complement and inflammation-associated genes used for SNP Discovery (Phase I). Particular attention was paid to genes known to participate in inflammation, immune-associated processes, coagulation/fibrinolysis and/or extracellular matrix homeostasis.

In choosing SNPs for these genes, a higher SNP density in the genic regions, which was defined as 5 Kb upstream from the start of transcription until 5 Kb downstream from the end of transcription, was applied. In these regions, an average density of 1 SNP per 10 Kb was used. In the non-genic regions of clusters of complement-related genes, an average of 1 SNP per 20 Kb was employed. The SNPs were chosen from HapMap data in the Caucasian population, the SNP Consortium (Marshall 1999, Science 284[5413]: 406-407), Whitehead, NCBI and the Celera SNP database. Selection included intronic SNPs, variants from the regulatory regions (mainly promoters) and coding SNPs (cSNPs) included in open reading frames. Data obtained by direct screening were used to validate the information extracted from databases. The overall sequence variation of functionally important regions of candidate genes was investigated, not merely a few polymorphisms, using a previously described algorithm for tag selection.

Positive controls included CEPH members (i.e., DNA samples derived from lymphoblastoid cell lines from 61 reference families provided to the NIGMS Repository by the Centre de'Etude du Polymorphism Humain (CEPH), Foundation Jean Dausset in Paris, France) of the HapMap trios; the nomenclature used for these samples is the Coriell sample name (i.e., family relationships were verified by the Coriell Institute for Medical Research Institute for Medical Research). The panel also contained a limited number of X-chromosome probes from two regions. These were included to provide additional information for inferring sample sex. Specifically, if the sample is clearly heterozygous for any X-chromosome markers, it must have two X-chromosomes. However, because there are a limited number of X-chromosome markers in the panel, and because their physical proximity likely means that there are even fewer haplotypes for these markers, we expected that samples with two X-chromosomes might also genotype as homozygous for these markers. The standard procedure for checking sample concordance involved two steps. The first step was to compare all samples with identical names for repeatability. In this study, the only repeats were positive controls and those had repeatability greater than 99.3% (range 99.85% to 100%). The second step was to compare all unique samples to all other unique samples and identify highly concordant sample pairs. Highly concordant sample pairs were used to identify possible tracking errors. The concordance test resulted in 20 sample pairs with concordance greater than 99%.

Samples were genotyped using multiplexed Molecular Inversion Probe (MIP) technology (ParAllele Biosciences/Affymetrix). Successful genotypes were obtained for 3,267 SNPs in 347 genes in 1113 unique samples (out of 1162 unique submitted samples; 3,267 successful assays out 3,308 assays attempted). SNPs with more than 5% failed calls (45 SNPs), SNPs with no allelic variation (354 alleles) and subjects with more than 5% missing genotypes (11 subjects) were deleted.

The resulting genotype data were analyzed in multiple sub-analyses, using a variety of appropriate statistical analyses, as described below.

Information on each polymorphic site indicating the sequence of SNP-associated alleles are shown in Table 4B. Specifically, Table 4B indicates the nucleotide present in Allele 1 and Allele 2 of each SNP. Tables 4C and 4D provide the flanking sequence information for some informative SNPs of the invention (Table 4C) and for certain MRD-designated SNPs (Table 4D). Further, certain SNPs presented in the Tables are identified by MRD designations in the parent U.S. provisional application No. 60/984,702. For example, in Table 1A, rs28362944 is also called MRD 4082.

A. Polymorphisms Associated with AAA:

One genotype association analysis was performed on all SNPs comparing samples derived from individuals with AAA to those derived from an ethnic- and age-matched control cohort. All genotype associations were assessed using a statistical software program known as SAS®. SNPs showing significant association with AAA are shown in Tables 1A and 2A.

Tables 1A and 2A provide allele and genotype frequency data for each SNP from which readily indicate to one of ordinary skill in the art whether each SNP is predisposing or protective for AAA. Table 2A contains a subset of polymorphisms that are risk-predictive for AAA and also risk-predictive forAMD (Table 2A). For example, a particular SNP can be considered to have informative or predictive value for a disease if the frequency of at least one allelic form is increased or decreased in the diseased population compared to a control population (e.g., a population of individuals known to lack the disease or not believed to be at any particular risk for the disease) and the difference is statistically significant. For example, an increased frequency of the minor allele (less frequent allele) in the diseased population compared to control population can be taken to indicate that the polymorphism is associated with increased risk for the disease (e.g., a predisposing polymorphism). A decreased frequency of the minor allele in the diseased population compared to control can be taken to indicate that the polymorphism is associated with decreased risk for the disease (e.g., a protective polymorphism).

Typically, the difference in frequencies between the diseased population and the control population is statistically significant. Statistical significance can be assessed using art-known methods, e.g., Chi Square, Fisher, Odds Ratio, Relative risk, Linkage Disequilibrium, Hardy-Weinberg equilibrium, genotype p-value and allelic p-value. For example, the statistically significant difference (increase or decrease) in distribution between diseased individuals and controls has a p-value (as determined by Genotype-Likelihood Ratio (3 categories) or a Chi Square test, or optionally both) of 0.1 or less. Optionally, SNPs with a p-value of less than 0.1 are considerend significant, those with a p-value of 0.05 or less are more significant, those with a p-value of 0.01 or less even more significant, and those with a p-value of 0.001 very significant. Optionally, the p-value is equal to or less than 0.1, 0.5, 0.01, 0.005, or 0.001 when determined by Genotype-Likelihood Ratio (3 categories), and is also equal to or less than 0.1, 0.5, 0.01, 0.005, or 0.001 when determined by Chi Square test. Optionally, the difference in allele frequency is optionally greater than 5%, between 5% and 10%, greater than 10%, between 10% and 20%, or greater than 20%, 30%, 40%, 60%, 70%, 80% or 90%.

For example, informative polymorphisms for AAA include rs3742089, rs2251252; and rs3737002. Additional informative polymorphisms include rs3764880 and rs4286111. Other informative polymorphisms include rs1126618, rs9943268, rs7416639, rs2227728, rs2227718, rs17259045, rs3814997, rs4657045, rs6003227, rs1859346, rs3742088, rs3756709, rs3751555, rs11580574, rs6875250, rs629275, rs10755538, rs7757078, rs536485, rs2230205, rs30300, rs4441274 and rs12906440. Still other informative polymorphisms include rs17013182, rs2072634, rs61917913 and rs34882957.

Other AAA-informative polymorphisms include polymorphisms that are predictive for both AAA and AMD, such as those discussed below. Some informative polymorphisms are MRD_{—}3991/rs2147021, MRD_{—}3996/rs34509370, MRD_{—}4008/rs12729569, MRD_{—}4082/rs28362944, rs10485243, rs11074715, rs11244834, rs11948133, rs12464480, rs12779767, rs1621212, rs1674923, rs1676717, rs1676736, rs1985671, rs2116142, rs3829467, rs4235376, rs4287571, rs4962543, rs554152, rs6064517, rs698086, and rs737330. These polymorphisms can be used to determine whether a individual has or is at risk of AAA. These polymorphisms can also be used to determine whether a individual has or is at risk of AAA or AMD or both. The individual optionally has at least one symptom or sign of AAA or AMD.

Other informative risk-predictive, e.g., predisposing or protective, polymorphisms are set forth in the Tables 2A and 2A.. In certain embodiments, the genetic profile comprises a combination of at least two SNPs selected from the pairs identified in Tables 3A and 3B.

B. Polymorphisms Associated with Elevated Risk for AAA and AMD:

In certain embodiments, one or more polymorphisms provided herein can have a statistically significant association with AAA and also with one or more disorders that involve dysfunction of the complement system. For example, an individual can have a genetic predisposition based on his/her genetic profile to AAA as well as a disorder associated with dysregulation of the complement system, such as AMD. The individual's genetic profile optionally comprises one or more polymorphisms shown in Tables 1A and 2A, wherein the genetic profile is informative of a combination of AAA and a complement-related disorder, e.g., AMD.

Table 2A include SNPs showing an association with a combination of AAA+AMD. These SNPs are thus associated with AAA in general, and can be used to assess risk not only of AAA in combination with AMD, but AAA in general.

Optionally the individual is known or suspected to have, or has at least one symptom or sign of AAA or AMD.

For example, some very useful risk-predictive polymorphisms for a combination of AAA and AMD (AAA+AMD) include rs12779767, rsl 1244834, rs1674923, rs1676736, rs10801554, rs1329421, and rs1071583. Additional highly useful risk-predictive polymorphisms for AAA+AMD include rs1676717, rs16891811, rs4505816, rsl 0485243, rs737330, rs3108966, rs3104052, and rs4684148. Still other useful predictive polymorphisms include rs1621212, rs6064517, rs6014959, rs28362944, rs4235376, rs7080536, rs331079, rs4657045, rs11580574, rs4385206, rs3012672, rs2986678, rs2986679, rs10846744, rs1463611, rs7658246, and rs9312522. Other risk-predictive polymorphisms are rs11575688 and rs1800888. Any combination of such SNPs can be used.

Other risk-predictive, e.g., predisposing or protective, polymorphisms are set forth in the Tables 1A or 2A or both.

Although the predictive value of the genetic profile can generally be enhanced by the inclusion of multiple SNPs, no one of the SNPs is indispensable. Accordingly, in various embodiments, one or more of the SNPs is omitted from the genetic profile.

In certain embodiments, the genetic profile comprises a combination of at least two SNPs selected from the pairs identified in Table 3B.

C. Genes Containing Polymorphisms Associated with AAA

In some embodiments, the screening incorporates one or more polymorphisms from genes having genetic variations correlating with a risk for AAA, including a combined risk for both AAA and AMD. Some such genes and SNPs disclosed in Tables 1A and 2A. Table 4A also provides gene identifiers based on the EnsEMBL database for some genes included in the invention. Thus the invention includes determining an individual's relative risk (i.e., susceptibility or propensity) of a particular vascular disorder by screening for the presence or absence of a genetic profile that includes one or more single nucleotide polymorphisms (SNPs) in at least one gene of interest. The presence of any one of the SNPs listed in Tables 1A and 2A is informative (i.e., indicative) of an individual's risk (increased or decreased) of the vascular disorder, or for predicting the course of progression of the disease in the individual. The vascular disorder is for example AAA, including AAA carrying an increased risk for AMD as well.

In an embodiment, the vascular disorder is AAA. Genes such as CR1, C1RL, SDC4, ADAM12, CFH, and FCN1 contained SNPs in strong association with AAA (e.g., a greater than 10% difference in genotype and/or allele frequency between diseased and control individuals, and/or a p-value<0.01). Genes such as TLR8, HS3ST4, C1QTNF7, COL19A1, FBLN2 and ENSG00000197467 (COL13A1) contained SNPs also in very high association with AAA (e.g., a 5-10% difference in frequency between diseased and control individuals, and/or a p-value<0.01). Genes such as ADAMTS19, APBA2, C3, C4BPA, FCGR2A, HS3ST4, ILGC1, RFX3, SPOCK, VTN, BMP7, C1NH, C1QTNF7, ENSG00000148702 (HABP2), FBN2, PPIC, SCARB 1 and SPOCK3 contained SNPs also in strong association with AAA (e.g., a 5-10% difference in frequency between diseased and control individuals, and/or a p-value<0.01). Genes such as IBSP/integrin-binding sialoprotein, C2-BF (factorB), ADRB2 and C9 contained SNPs also in high association with AAA (e.g., a 5-10% difference in frequency between diseased and control individuals, and/or a p-value<0.01). Other genes with AAA-associated SNPs are listed in Tables 1A and 2A, with additional raw data provided in Tables 1B and 2B. In an embodiment, the individual is screened for any combination of these genes.

Useful SNPs for the gene ADAMTS19 include rs25816, rs6875250, rs25821, rs30300, rs10072248, rs10070537, or rs30693. Especially useful SNPs include rs rs30300 or rs6875250. Useful SNPs for the gene APBA2 include rs12906440 or rs3751555. Useful SNPs for the gene C1RL include rs3742088, rs61917913, rs744141or rs3742089. Especially useful SNPs include rs3742089. Other useful SNPs include rs3742088 and rs61917913 Useful SNPs for the gene C2-BF(factorB) include rs2072634. Useful SNPs for the gene C3 include rs2230205. Useful SNPs for the gene C4BPA include rs1126618, rs9943268 or rs7416639. Useful SNPs for the gene C9 include rs34882957. Useful SNPs for the gene COL19A1 include rs10755538, rs7757078, rs2502560 or rs1340975. Useful SNPs for the gene CR1 include rs3737002, rs17259045, rs4844599, not known, rs1408078, rs2274567 or rs11118167. Especially useful SNPs include rs3737002, or rs17259045. Useful SNPs for the gene ENSG00000029559 (IBSP/integrin-binding sialoprotein) include rs17013182. Useful SNPs for the gene FCGR2A include rs4657045 or rs11580574. Useful SNPs for the gene HS3ST4 include rs4441276, rs4286111, rs4441274, rs11645232, rs6497910 or rs12103080. Especially useful SNPs include rs4441274 and rs4286111. Useful SNPs for the gene IGLC1 include rs3814997 or rs6003227. Useful SNPs for the gene RFX3 include rs629275, rs536485, rs559746 or rs613518 include rs629275 or rs536485. Useful SNPs for the gene SDC4 include rs2251252. Useful SNPs for the gene SPOCK include rs1859346, rs3756709, rs2905965, rs2905972, rsl 1948133, rs10491299, rs12719499 or rs6873075. Especially useful SNPs include rs1859346 or rs3756709. Useful SNPs for the gene TLR8 include rs5978593, rs3764880, rs3827469, rs5741883 or rs1013150. Especially useful SNPs include rs3764880. Useful SNPs for the gene VTN include rs2227728 or rs2227718. Any combination of SNPs can be used.

D. Genes Containing Polymorphisms Associated with Both AAA and AMD

A subset of individuals suffering from AAA have also been found to suffer from AMD. As mentioned, the invention includes determining an individual's relative risk (i.e., susceptibility or propensity) of a combination of AAA and AMD by screening for the presence or absence of a genetic profile that includes one or more single nucleotide polymorphisms (SNPs) in at least one gene of interest. The presence of any one of the SNPs listed in Table 2A is especially informative (i.e., indicative) of an individual's risk (increased or decreased) of a combination of AAA and AMD (AAA+AMD), or for predicting the course of progression of AAA+AMD in the individual. SNPs of Table 1A can also be used.

Genes such as ADAM12, ‘ENSG00000000971 (CFH) and FCN1 contained SNPs in strong association with AAA+AMD (e.g., a greater than 10% difference in frequency between diseased and control individuals, and/or a p-value<0.01). Genes such as C1QTNF7, COL19A1, ‘ENSG00000197467 (COL13A1) and FBLN2 contained SNPs also in very high association with AAA+AMD (e.g., a 5-10% difference in frequency between diseased and control individuals, and/or a p-value<0.01). Genes such as BMP7, CINH, ENSG00000148702 (HABP2), FBN2, FCGR2A, PPIC, RFX3 , SCARB1and SPOCK3 contained SNPs also in strong association with AAA+AMD (e.g., a 5-10% difference in frequency between diseased and control individuals, and/or a p-value<0.01). Genes such as ADRB2 contained SNPs also in high association with AAA+AMD (e.g., a 5-10% difference in frequency between diseased and control individuals, and/or a p-value<0.01). Other genes with AAAA+AMD-associated SNPs are listed in Tables 1A and 2A, with additional raw data provided in Tables 1B and 2B. In an embodiment, the individual is screened for polymorphisms in any combination of these genes.

Useful SNPs for the gene ADAM12 include rs4962543, rs1621212, rs1676717, rs12779767, rsl 1244834, rs1674923, rs1676736, rs4130179, or rs1674888. Useful SNPs for the gene ADRB2 include rs1800888. These are especially useful for AAA+AMD. ADAM 12, also known as A disintegrin and metalloproteinase domain 12 (GenBank Accession Nos. CAI40682 and CAI40683), is a member of the ADAM (a disintegrin and metalloprotease) protein family that contains pro-, metalloprotease, disintegrin, cysteine-rich, transmembrane and cytoplasmic domains. Members of the ADAM family are membrane-anchored proteins structurally related to snake venom metalloproteases (SVMPs), and have been implicated in a variety of biological processes including modulating proteolysis, signaling, cell-cell and cell-matrix interactions, cell fusion, fertilization, muscle development, and neurogenesis. ADAM12 is involved in skeletal muscle regeneration, specifically at the onset of cell fusion, and in the formation of macrophage-derived giant cells (MGC) and osteoclasts from mononuclearprecursors. ADAM12 is an active metalloprotease, and has been implicated in insulin-like growth factor (IGF) receptor signaling through cleavage of IGF-binding proteins and in epidermal growth factor receptor (EGFR) pathways, through ectodomain shedding of membrane-tethered EGFR ligands. These proteolytic events can regulate diverse cellular responses, such as altered cell differentiation, proliferation, migration, and invasion. ADAM12 can also regulate cell-cell and cell-extracellular matrix contacts through interactions with cell surface receptors, such as integrins and syndecans, potentially influencing the actin cytoskeleton. Moreover, ADAM 12 interacts with several cytoplasmic signaling and adaptor molecules through its intracellular domain, thereby directly transmitting signals to or from the cell interior. ADAM 12 has also emerged as biomarker for human breast cancer. (See, e.g., Gilpin, et al., Journal of Biological Chemistry 273(1):157-166 (1998) and Dyczynska, et al., International Journal of Cancer, 122(11):2634-2640 (2008).)

Useful SNPs for the gene BMP7 include rs6064517, rs6014959, rs6064506, rs6025422, rs6127984, rs8116259, rs162315, or rs162316. Especially useful SNPs include rs6064517 and rs6014959. Useful SNPs for the gene CINH include rs28362944. Useful SNPs for the gene C1QTNF7include rs4235376, rs13116208, rs16891811, rs4698382, rs4505816, rs2192356, or rs2215809. Especially useful SNPs include Useful SNPs for the gene COL19A1 include rs10485243, rs737330, or rs2145905. Useful SNPs for the gene ENSG00000000971 (CFH) include rs1329421, or rs10801554. Useful SNPs for the gene ENSG00000148702 (HABP2)include rs7080536, or rs11575688. Useful SNPs for the gene ENSG00000197467include rs3108966, or rs3104052. Useful SNPs for the gene FBLN2 include rs4684148. Useful SNPs for the gene FBN2 include rs331079, rs10073062, rs27913, or rs468182. Especially useful SNPs include rs331079. Useful SNPs for the gene FCGR2A include rs4657045, or rs11580574. Useful SNPs for the gene FCN1 include rs1071583, or rs2989727. Useful SNPs for the gene PPIC include rs4385206. Useful SNPs for the gene RFX3 include rs3012672, rs2986678, or rs2986679. Useful SNPs for the gene SCARB1 include rs10846744, Useful SNPs for the gene SPOCK3 include rs1463611, rs7658246, rs1579404, rs9312522, or rs9996643include rs1463611, rs7658246, rs9312522. Especially useful SNPs include rs1463611, rs7658246 and rs9312522.

CR1, also known human C3b/C4b receptor or complement receptor type one (GenBank Accession No. CAI16044), is a single chain membrane glycoprotein that plays an important role in immune complex processing. The CR1 family of receptor and regulatory glycoproteins are composed of a tandemly repeated motif (short consensus repeat, SCR, or Sushi elements) of 59-72 amino acid residues in length. CR1 features an internal homology region of seven SCRs in length, known as a long homologous repeat, that is reiterated four times in predominant polymorphic size variant. For other polymorphic forms of CR1, the region can be reiterated three, five and six times. This repeated motif is characteristic of a number of C3b- and C4b-binding proteins that are involved in the control of complement activation. (See, e.g., Hourcade, et al., Journal of Biological Chemistry 265(2):974-980 (1990) and Logar, et al., Molecular Immunology 40(11):831-840 (2004).)

C1RL, also known as complement component 1, r subcomponent-like (GenBank Accession Nos. AAH62428 and NP_{—}057630), encodes for C1r-like serine protease analog, CLSPa, derived from dendritic cells (DC). C1RL shares great homology with complement C1r/C1s and mannose-associated serine proteases. C I RL mRNA is widely expressed, especially abundant in placenta, liver, kidney, pancreas, and myeloid cells, which are a major resources of serine proteases.

C4BPA (complement component 4 binding protein, α, also called C4b binding protein, α chain, NCBI Ref. NM_{—}000715.3; Ensembl:ENSG00000123838; HPRD:00403; MIM:120830) controls the classical pathway of complement activation. It binds as a cofactor to C3b/C4b inactivator (C3bINA), which then hydrolyzes the complement fragment C4b. It also accelerates the degradation of the C4bC2a complex (C3 convertase) by dissociating the complement fragment C2a. Alpha chain binds C4b. It interacts also with anticoagulant protein S and with serum amyloid P component

SDC4, also known as syndecan 4 (GenBank Accession Nos. CAG46871, CAG46842, and NP_{—}002990), is a member of the syndecan family of cell surface receptors that participate in cell-cell and cell-matrix interactions important for development. SDC4 exhibits pro-angiogenic pathway functions by contributing to endothelial tubulogenesis through its interactions with thrombospondin-1 (TSP-1). Further, SDC4 is an intrinsic regulator of inflammatory reactions through its effects on osteopontin (OPN) function. (See, e.g., Nunes, et al., Journal of Cellular Physiology 214(3):828-837 (2008); Kon, et al., The Journal of Experimental Medicine 205(1):25-33 (2008); and Dews, et al., PNAS 104(52):20782-20787 (2007).)

CFH, also known as complement factor H or complement regulatory genes factor H (GenBank Accession Nos. NP_{—}000177, NG_{—}007259 and NM_{—}000186), is a member of the regulator of complement activation (RCA) gene cluster and encodes a protein with twenty short consensus repeat (SCR) domains. The CFH protein is secreted into the bloodstream and has an essential role in the regulation of complement activation, restricting this innate defense mechanism to microbial infections. Mutations in this gene have been associated with hemolytic-uremic syndrome (HUS), chronic hypocomplementemic nephropathy and Membranoproliferative glomerulonephritis type II or dense deposit disease (MPGN II/DDD), age-related macular degeneration (AMD) and colon cancer.

FCN1, also known as ficolin (collagen/fibrinogen domain containing) 1 or M-Ficolin (GenBank Accession Nos. NM_{—}002003 and NP_{—}001994) is a member of the ficolin family of proteins which are characterized by the presence of a leader peptide, a short N-terminal segment, followed by a collagen-like region, and a C-terminal fibrinogen-like domain. The FCN1 protein is pattern recognition molecule of the complement system and is predominantly expressed in the peripheral blood leukocytes, myeloid cells and type II alveolar epithelial cells. FCN1 has been postulated to function as a plasma protein with elastin-binding activity.

HS3ST4 encodes the enzyme heparan sulfate D-glucosaminyl 3-O-sulfotransferase 4, also known as 3-OST-4 (Genbank Accession Nos. ABN79919, ABN79918, ABN79917, ABN79916, ABN79915, ABN79914, ABN79913 and ABN79912). HS3ST4 generates 3-O-sulfated glucosaminyl residues in heparan sulfate by transfer of a sulfuryl group to an N-unsubstituted glucosamine linked to a 2-O-sulfo iduronic acid unit on heparan sulfate. Unlike 3-OST-1, HS3ST4 does not convert non-anticoagulant heparin sulfate to anticoagulant heparan sulfate.

TLR7, also known as toll-like receptor 7 (GenBank Accession No. NP_{—}057646), is a member of the Toll-like receptor (TLR) family which plays a fundamental role in pathogen recognition and activation of innate immunity. Specifically, TLR7 participates in the innate immune response to microbial agents and functions via IVIYD88 and TRAF6, leading to NF-kappa-B activation, cytokine secretion and the inflammatory response. (See, e.g., Du, et al., European Cytokine Network 11(3):362-371 (2000); Horsmans, et al., Hepatology 42(3):724-731 (2005); Hemmi, et al., Nature Immunology 3(2):196-200 (2002); and Chuang, et al., European Cytokine Network 11(3):372-378 (2000)).

TLR8, also know as toll-like receptor 8 (GenBank Accession Nos. NP_{—}619542 and AAQ88663), is a member of the Toll-like receptor (TLR) family which plays a fundamental role in pathogen recognition and activation of innate immunity. Specifically, TLR8 participates in the innate immune response to microbial agents and functions via MYD88 and TRAF6, leading to NF-kappa-B activation, cytokine secretion and the inflammatory response. (See, e.g., Du, et al., European Cytokine Network 11(3):362-371 (2000); Cheng, et al., Translational Research 150(5):311-318 (2007); Jeffries, et al., Journal of Biological Chemistry 278(28):26258-26264 (2003); Peng, et al., Science 309(5739):1380-1384 (2005); and Chuang, et al., European Cytokine Network 11(3):372-378 (2000)).

C1QTNF7, also known as C1q and tumor necrosis factor related protein 7 or complement-c1q tumor necrosis factor-related protein 7 (GenBank Accession Nos. NP_{—}114117 and NP_{—}001128643), was identified during the National Institutes of Health's Mammalian Gene Collection (MGC) project by homology-based searches for TNF paralogs. C1QTNF7 (C1Q/TNF7) is a C1q domain-containing protein that also shares homology with TNF alpha. Overexpression of murine C1qTNF3 can enhance the cell growth/proliferation, indicating it functions as a growth factor; C1Q/TNF7 can exhibit similar properties.

COL13A1, also know as alpha 1 type XIII collagen 2, collagen alpha-1(XIII) chain 2, or collagen type XIII alpha 1 (ENSG00000197467; GenBank Accession Nos. CAI15451, CAI15452 and CAI15450), encodes the alpha chain of one of the nonfibrillar collagens.

COL19A1, also known as collagen alpha-1(Y) chain 3, a1 chain of type XIX collagen 2, alpha 1 type XIX collagen 2, or collagen XIX alpha 1 (GenBank Accession Nos. CAI42319, CAC12699, CAI42322, CAI42716, CAI42497 and NP_{—}001849), is a member of the FACIT collagen family (fibril-associated collagens with interrupted helices).

FBLN1, also known as fibulin 1, (GenBank Accession Nos. NP_{—}001987, CAQ10154, CAQ10155 and CAQ10153.1), is a secreted glycoprotein that becomes incorporated into a fibrillar extracellular matrix. Calcium-binding is required to mediate FBLN1 protein binding to laminin and nidogen. It mediates platelet adhesion via binding fibrinogen. FBLN 1 protein can be important for developmental processes, as well as contributing to the supramolecular organization of ECM architecture, in particular to architecture of basement membranes. FBLN1 protein can also play a role in haemostasis and thrombosis due to its ability to bind fibrinogen and incorporate into blood clots.

FBLN2, also known as fibulin 2 (GenBank Accession No. NP_{—}001004019, AAN05436, AAN05435 and NP_{—}001989), encodes an extracellular matrix protein that belongs to the fibulin family. FBLN2 protein binds various extracellular ligands and calcium, and the binding of FBLN2 to fibronectin and some other ligands has been shown to be calcium dependent.

ITGA6, also know as integrin alpha-6 (GenBank Accession Nos. NP_{—}000201, NP_{—}001073286 and AAHSO585.1), is a member of the integrin family of proteins. Integrins are integral cell-surface proteins composed of an alpha chain and a beta chain. ITGA6 has been found to modulate cell migration during tumor cell invasion and migration, and has been found to be involved with metastasis in a variety of tumors including prostate, liver, gastrointestinal and pancreatic cancers. (See, e.g., Pawar, et al., Experimental Cell Research 313(6):1080-1089 (2007); Hogervorst, et al., The Journal of Cellular Biology 121(1):179-191 (1993); Gulubova, Clinical & Experimental Metastasis 21(6):485-494 (2004); and Lipscomb, et al., Cancer and Metastasis Reviews 24:413-423 (2005).)

### V. Determination of Risk (Screening):

### Determining the Risk of an Individual

The presence in the genome or transcriptome of an individual of one or more polymorphisms listed in Tables IA and 2A is associated with an increased or decreased risk of AAA in general. Accordingly, detection of a polymorphism shown in Tables 1A and 2A in a nucleic acid sample of an individual can indicate that the individual is at increased risk for AAA.

One of skill in the art may refer to Tables 1A and 2A to identify alleles associated with increased (or decreased) likelihood of development and/or progression of AAA. The genotypes depicted in the Tables are organized alphabetically by gene symbol. SNPs identified in a given gene are designated by SNP number (rs#). Table 4B provides information regarding the allelic variation of each SNP, and specifically indicates the nucleotide present at the polymorphic site in either allele 1 or allele 2. For example, Table 4B indicates that allele 1 of the SNP rs30300 in the ADAMST19 gene has a “C” base at the polymorphic site, while allele 2 has a “T” base at this position. The frequencies for both allele 1 and allele 2 are shown in Tables 1A and 2A as percentages in both control and disease populations for individuals homozygous for allele 1, individuals homozygous for allele 2, and heterozygous individuals. For example, Table 1A indicates that the SNP rs30300 is located in the ADAM metallopeptidase with thrombospondin type 1 motif, 19 (ADAMTS19) gene, 1.4% of the control population is homozygous for allele 1 (i.e., the allele which has an “C” base at this position), 77% of the control population is homozygous for allele 2 (i.e., the allel which has a “T” base at this position), and 21.6% of the control population is heterozygous. The overall frequency for allele 2, which is the more frequent (“wild-type”) allele (i.e., the “T” allele) in the control population is 87.8% and the overall frequency for allele 1 in the control population is 12.2%. In the AAA population, 0% of the population is homozygous for allele 1 (the “C” allele), 89.5% of population is homozygous for allele 2 (the “T” allele), and 10.5% of the population is heterozygous. The overall frequency for allele 1 (the “C” allele) in the AAA population is 5.3% and the overall frequency for allele 2 (the “T” allele) in the AAA population is 94.7%. Genotype-Likelihood Ratio (3 categories) and Chi Square values (“Freq. Chi Square (both collapsed-2 categories)”) are provided for each SNP. Table 1A thus indicates that a person having allele 1 has a lesser likelihood of developing AAA than a person not having allele 1 (See Tables 1A and 2A). Allele 2, which is the “T” allele, is the more common allele (i.e. the “wild type” allele). Allele 1, which is the “C” allele, is the rarer allele and is more prevalent in the control population than in the AMD population: it is therefore a “protective polymorphism.” Tables 1A and 2A provide the raw data from which the percentages of allele frequencies as shown in Tables 1A and 2A were calculated.

Similarly, the presence in the genome or transcriptome of an individual of one or more polymorphisms listed in Tables 2A is associated with an increased or decreased risk of AAA as well as AMD (“AAA+AMD”). Accordingly, detection of a polymorphism shown in Table 2A in a nucleic acid sample of an individual can indicate that the individual is at increased risk for AAA+AMD. One of skill in the art will be able to refer to Table 2A to identify alleles associated with increased (or decreased) likelihood of AAA+AMD. Optionally, one or more polymorphisms from Table 1A can be used in determining the risk of AAA+AMD.

In other embodiments, the presence of a combination of multiple (e.g., two or more, three or more, four or more, or five or more) AAA+AMD-associated polymorphisms shown in Tables 1A and 2A indicates an increased (or decreased) risk for AAA+AMD.

An individual's relative risk (i.e., susceptibility or propensity) of a particular vascular disorder can be determined by screening for the presence or absence of a genetic profile that includes one or more single nucleotide polymorphisms (SNPs) selected from Tables 1A and 2A. The vascular disorder is preferably AAA. The presence of any one of the SNPs listed in Tables 1A and 2A is informative (i.e., indicative) of an individual's risk (increased or decreased) of having or developing AAA, or for predicting the course of progression of in the individual. Optionally, the individual's relative risk of both AAA and AMD in combination can be determined using one or more polymorphisms in Table 2A.

The predictive value of a genetic profile for AAA can be increased by screening for a combination of SNPs selected from Tables 1 A and 2A. In one embodiment, the predictive value of a genetic profile is increased by screening for the presence of at least 2 SNPs, at least 3 SNPs, at least 4 SNPs, at least 5 SNPs, at least 6 SNPs, at least 7 SNPs, at least 8 SNPs, at least 9 SNPs, or at least 10 SNPs selected from Tables 1A and 2A.

The predictive value of a genetic profile for AAA (including AAA in combination with AMD) can also be increased by screening for a combination of predisposing and protective polymorphisms. For example, the absence of at least one, typically multiple, predisposing polymorphisms and the presence of at least one, typically multiple, protective polymorphisms can indicate that the individual is not at risk of AAA. Alternatively, the presence of at least one, typically multiple, predisposing SNPs and the absence of at least one, typically multiple, protective SNPs indicate that the individual is at risk of AAA.

In a further embodiment, the determination of an individual's genetic profile can also include screening for a deletion (e.g., a heterozygous deletion) that is associated with AAA risk. Exemplary deletions that are associated with AAA risk include a deletion in FHR3 and FHR1 genes. See, e.g., International Pub. No. WO 2008/013893, incorporated by reference in its entirety. The deletion can encompass one gene, multiple genes, a portion of a gene, or an intergenic region, for example. If the deletion impacts the size, conformation, expression or stability of an encoded protein, the deletion can be detected by assaying the protein, or by querying the nucleic acid sequence of the genome or transcriptome of the individual.

Further, determining an individual's genetic profile can include determining an individual's genotype or haplotype to determine if the individual is at an increased or decreased risk of AAA. In one embodiment, an individual's genetic profile can comprise SNPs that are in linkage disequilibrium with other SNPs associated with AAA that define a haplotype associated with risk or protection of AAA. In another embodiment, a genetic profile can include multiple haplotypes present in the genome or a combination of haplotypes and polymorphisms, such as single nucleotide polymorphisms, in the genome. Optionally, the genetic profile comprises one or more polymorphisms that indicate an increased or decreased risk for contracting both AAA and AMD.

Further studies of the identity of the various SNPs and other genetic characteristics disclosed herein with additional cohorts, and clinical experience with the practice of this invention on populations, will permit ever more precise assessment of AAA risk based on emergent SNP patterns. This work will result in refinement of which particular set of SNPs are characteristic of a genetic profile which is, for example, indicative of an urgent need for intervention, or indicative that the early stage of AAA observed in an individual is unlikely to progress to more serious disease, or is likely to progress rapidly to the wet form of the disease, or that the presenting individual is not at significant risk of AAA, or that a particular AAA therapy is most likely to be successful with this individual and another therapeutic alternative less likely to be productive. Thus, it is anticipated that the practice of the invention disclosed herein, especially when combined with the practice of risk assessment using other known risk-indicative and protection-indicative SNPs, will permit disease management and avoidance with increasing precision.

A single nucleotide polymorphism within a genetic profile as described herein can be detected directly or indirectly. Direct detection refers to determining the presence or absence of a specific SNP identified in the genetic profile using a suitable nucleic acid, such as an oligonucleotide in the form of a probe or primer as described below. Alternatively, direct detection can include querying a pre-produced database comprising all or part of the individual's genome for a specific SNP in the genetic profile. Other direct methods are known to those skilled in the art. Indirect detection refers to determining the presence or absence of a specific SNP identified in the genetic profile by detecting a surrogate or proxy SNP that is in linkage disequilibrium with the SNP in the individual's genetic profile. Detection of a proxy SNP is indicative of a SNP of interest and is increasingly informative to the extent that the SNPs are in linkage disequilibrium, e.g., at least 50%, 60%, 70%, 80%, 90%, 95%, 98%, or about 100% LD. Another indirect method involves detecting allelic variants of proteins accessible in a sample from an individual that are consequent of a risk-associated or protection-associated allele in DNA that alters a codon.

It is also understood that a genetic profile as described herein can include one or more nucleotide polymorphism(s) that are in linkage disequilibrium with a polymorphism that is causative of disease. In this case, the SNP in the genetic profile is a surrogate SNP for the causative polymorphism.

### Detection of SNPs

A disease-associated genetic profile, such as one that is associated with AAA, may comprise multiple, genetically-linked SNPs. Genetically-linked SNPs may be identified by art known methods. Non-random associations between polymorphisms (including single nucleotide polymorphisms, or SNPs) at two or more loci are measured by the degree of linkage disequilibrium (LD). The degree of linkage disequilibrium is influenced by a number of factors including genetic linkage, the rate of recombination, the rate of mutation, random drift, non-random mating and population structure. Moreover, loci that are in LD do not have to be located on the same chromosome, although most typically they occur as clusters of adjacent variations within a restricted segment of DNA. Polymorphisms that are in complete or close LD with a particular disease-associated SNP are also useful for screening, diagnosis, and the like.

SNPs in LD with each other can be identified using art-known methods and SNP databases (e.g., the Perlegen database, at http://genome.perlegen.com/browser/download.html and others). For illustration, SNPs in linkage disequilibrium (LD) with the rs3737002 were identified using the Perlegen database. This database groups SNPs into LD bins such that all SNPs in the bin are highly correlated to each other. For example, AMD-associated SNP rs800292 was identified in the Perlegen database under the identifier ‘afd0678310’. A LD bin (see table below) was then identified that contained linked SNPs—including afd 1007146, afd1168124, afd1167840, and afd1167838—and annotations

| CR1 | | | | | | Allele |
|---|---|---|---|---|---|---|
| SNP ID | | | | | | Frequency |
| Perlegen | | SNP Position | | | | European |
| ‘afd’ ID* | ss ID | Chromosome | Accession | Position | Alleles | American |
| afd1007146 | ss23852667 | 1 | NC_000001.5 | 33394811 | T/C | 0.88 |
| afd1168124 | ss24141999 | 1 | NC_000001.5 | 204844472 | C/T | 0.9 |
| afd1167840 | ss24142016 | 1 | NC_000001.5 | 204958403 | G/A | 0.88 |
| afd1167838 | ss24142017 | 1 | NC_000001.5 | 204958874 | G/T | 0.85 |

*Perlegen AFD identification numbers can be converted into conventional SNP database identifiers (in this case, rs16835467, rs3737002, rs17049197, and rs4844614 in both the CR1 and CR1L genes) using the NCBI database (http://www.ncbi.nlm.nih.gov/sites/entrez?db=snp&cmd=search&term=).

The frequencies of these alleles in disease versus control populations may be determined using the methods described herein.

As a second example, the LD tables computed by HapMap may be downloaded (http://ftp.hapmap.org/ld_data/latest/) and used. Unlike the Perlegen database, the HapMap tables use ‘rs’ SNP identifiers directly. For illustration, the table below shows SNPs in LD with rs800292 (a SNP in the complement factor h gene used here for illustration). All SNPs with an R² value greater than 0.80 were extracted from the database in this illustration.

| | SNP #2 | | | | | | |
|---|---|---|---|---|---|---|---|
| SNP 1 Location | Location | Population | SNP #1 ID | SNP #2 ID | D′ | R² | LOD |
| 194846662 | 194908856 | CEU | rs10801551 | rs800292 | 1 | 0.84 | 19.31 |
| 194850944 | 194908856 | CEU | rs4657825 | rs800292 | 1 | 0.9 | 21.22 |
| 194851091 | 194908856 | CEU | rs12061508 | rs800292 | 1 | 0.83 | 18.15 |
| 194886125 | 194908856 | CEU | rs505102 | rs800292 | 1 | 0.95 | 23.04 |
| 194899093 | 194908856 | CEU | rs6680396 | rs800292 | 1 | 0.84 | 19.61 |
| 194901729 | 194908856 | CEU | rs529825 | rs800292 | 1 | 0.95 | 23.04 |
| 194908856 | 194928161 | CEU | rs800292 | rs12124794 | 1 | 0.84 | 18.81 |
| 194908856 | 194947437 | CEU | rs800292 | rs1831281 | 1 | 0.84 | 19.61 |
| 194908856 | 194969148 | CEU | rs800292 | rs2284664 | 1 | 0.84 | 19.61 |
| 194908856 | 194981223 | CEU | rs800292 | rs10801560 | 1 | 0.84 | 19.61 |
| 194908856 | 194981293 | CEU | rs800292 | rs10801561 | 1 | 0.84 | 19.61 |
| 194908856 | 195089923 | CEU | rs800292 | rs10922144 | 1 | 0.84 | 19.61 |

Thus, publicly available databases such as the HapMap database (http://ftp.hapmap.org/ld_data/latest/), Haploview (Barrett, J. C. et al., Bioinformatics 21, 263 (2005)), and Perlegen (http://genome.perlegen.com/browser/download.html) can be used to calculate linkage disequilibiurm between two SNPs. The frequency of these alleles in disease versus control populations can be determined using the methods described herein. Statistical analyses can be employed to determine the significance of a non-random association between the two SNPs (e.g., Hardy-Weinberg Equilibrium, Genotype likelihood ratio (genotype p value), Chi Square analysis, Fishers Exact test). A statistically significant non-random association between the two SNPs indicates that they are in linkage disequilibrium and that one SNP can serve as a proxy for the second SNP.

The screening step to determine an individual's genetic profile can be conducted by inspecting a data set indicative of genetic characteristics previously derived from analysis of the individual's genome. A data set indicative of an individual's genetic characteristics can include a complete or partial sequence of the individual's genomic DNA, or a SNP map. Inspection of the data set including all or part of the individual's genome can optimally be performed by computer inspection. Screening can further comprise the step of producing a report identifying the individual and the identity of alleles at the site of at least one or more polymorphisms shown in Tables 1A and 2A.

Alternatively, the screening step to determine an individual's genetic profile includes analyzing a nucleic acid (i.e., DNA or RNA) sample obtained from the individual. A sample can be from any source containing nucleic acids (e.g., DNA or RNA) including tissues such as hair, skin, blood, biopsies of the retina, kidney, or liver or other organs or tissues, or sources such as saliva, cheek scrapings, urine, amniotic fluid or CVS samples, and the like. Typically, genomic DNA is analyzed. Alternatively, RNA, cDNA, or protein can be analyzed. Methods for the purification or partial purification of nucleic acids or proteins from a sample, and various protocols for analyzing samples for use in diagnostic assays are well known.

A polymorphism such as a SNP can be conveniently detected using suitable nucleic acids, such as oligonucleotides in the form of primers or probes. Accordingly, the invention not only provides novel SNPs and/or novel combinations of SNPs that are useful in assessing risk for a vascular disorder, but also nucleic acids such as oligonucleotides useful to detect them. A useful oligonucleotide for instance comprises a sequence that hybridizes under stringent hybridization conditions to at least one polymorphism identified herein. Where appropriate, at least one oligonucleotide includes a sequence that is fully complementary to a nucleic acid sequence comprising at least one polymorphism identified herein. Such oligonucleotide(s) can be used to detect the presence of the corresponding polymorphism, for example by hybridizing to the polymorphism under stringent hybridizing conditions, or by acting as an extension primer in either an amplification reaction such as PCR or a sequencing reaction, wherein the corresponding polymorphism is detected either by amplification or sequencing. Suitable detection methods are described below.

An individual's genotype can be determined using any method capable of identifying nucleotide variation, for instance at single nucleotide polymorphic sites. The particular method used is not a critical aspect of the invention. Although considerations of performance, cost, and convenience will make particular methods more desirable than others, it will be clear that any method that can detect one or more polymorphisms of interest can be used to practice the invention. A number of suitable methods are described below.

1) Nucleic Acid Analysis

General

Polymorphisms can be identified through the analysis of the nucleic acid sequence present at one or more of the polymorphic sites. A number of such methods are known in the art. Some such methods can involve hybridization, for instance with probes (probe-based methods). Other methods can involve amplification of nucleic acid (amplification-based methods). Still other methods can include both hybridization and amplification, or neither.

a) Amplification-Based Methods

Preamplification Followed by Sequence Analysis:

Where useful, an amplification product that encompasses a locus of interest can be generated from a nucleic acid sample. The specific polymorphism present at the locus is then determined by further analysis of the amplification product, for instance by methods described below. Allele-independent amplification can be achieved using primers which hybridize to conserved regions of the genes. The genes contain many invariant or monomorphic regions and suitable allele-independent primers can be selected routinely.

Upon generation of an amplified product, polymorphisms of interest can be identified by DNA sequencing methods, such as the chain termination method (Sanger et al., 1977, Proc. Natl. Acad. Sci,. 74:5463-5467) or PCR-based sequencing. Other useful analytical techniques that can detect the presence of a polymorphism in the amplified product include single-strand conformation polymorphism (SSCP) analysis, denaturing gradient gel electropohoresis (DGGE) analysis, and/or denaturing high performance liquid chromatography (DHPLC) analysis. In such techniques, different alleles can be identified based on sequence- and structure-dependent electrophoretic migration of single stranded PCR products. Amplified PCR products can be generated according to standard protocols, and heated or otherwise denatured to form single stranded products, which can refold or form secondary structures that are partially dependent on base sequence. An alternative method, referred to herein as a kinetic-PCR method, in which the generation of amplified nucleic acid is detected by monitoring the increase in the total amount of double-stranded DNA in the reaction mixture, is described in Higuchi et al., 1992, Bio/Technology, 10:413-417, incorporated herein by reference.

Allele-Specific Amplification:

Alleles can also be identified using amplification-based methods. Various nucleic acid amplification methods known in the art can be used in to detect nucleotide changes in a target nucleic acid. Alleles can also be identified using allele-specific amplification or primer extension methods, in which amplification or extension primers and/or conditions are selected that generate a product only if a polymorphism of interest is present.

Amplification Technologies

A preferred method is the polymerase chain reaction (PCR), which is now well known in the art, and described in U.S. Pat. Nos. 4,683,195; 4,683,202; and 4,965,188; each incorporated herein by reference. Other suitable amplification methods include the ligase chain reaction (Wu and Wallace, 1988, Genomics 4:560-569); the strand displacement assay (Walker et al., 1992, Proc. Natl. Acad. Sci. USA 89:392-396, Walker et al. 1992, Nucleic Acids Res. 20:1691-1696, and U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., 1989, Proc. Natl. Acad. Sci. USA, 86:1173-1177); and self-sustained sequence replication (3SR) (Guatelli et al., 1990, Proc. Natl. Acad. Sci. USA, 87:1874-1878 and WO 92/08800); each incorporated herein by reference. Alternatively, methods that amplify the probe to detectable levels can be used, such as QB-replicase amplification (Kramer et al., 1989, Nature, 339:401-402, and Lomeli et al., 1989, Clin. Chem., 35:1826-1831, both of which are incorporated herein by reference). A review of known amplification methods is provided in Abramson et al., 1993, Current Opinion in Biotechnology, 4:41-47, incorporated herein by reference.

Amplification of mRNA

Genotyping also can also be carried out by detecting and analyzing mRNA under conditions when both maternal and paternal chromosomes are transcribed. Amplification of RNA can be carried out by first reverse-transcribing the target RNA using, for example, a viral reverse transcriptase, and then amplifying the resulting cDNA, or using a combined high-temperature reverse-transcription-polymerase chain reaction (RT-PCR), as described in U.S. Pat. Nos. 5,310,652; 5,322,770; 5,561,058; 5,641,864; and 5,693,517; each incorporated herein by reference (see also Myers and Sigua, 1995, in PCR Strategies, supra, chapter 5).

Selection of Allele-Specific Primers

The design of an allele-specific primer can utilize the inhibitory effect of a terminal primer mismatch on the ability of a DNA polymerase to extend the primer. To detect an allele sequence using an allele-specific amplification or extension-based method, a primer complementary to the genes of interest is chosen such that the nucleotide hybridizes at or near the polymorphic position. For instance, the primer can be designed to exactly match the polymorphism at the 3′ terminus such that the primer can only be extended efficiently under stringent hybridization conditions in the presence of nucleic acid that contains the polymorphism. Allele-specific amplification- or extension-based methods are described in, for example, U.S. Pat. Nos. 5,137,806; 5,595,890; 5,639,611; and U.S. Pat. No. 4,851,331, each incorporated herein by reference.

Analysis of Heterozygous Samples

If so desired, allele-specific amplification can be used to amplify a region encompassing multiple polymorphic sites from only one of the two alleles in a heterozygous sample.

b) Probe-Based Methods:

General

Alleles can be also identified using probe-based methods, which rely on the difference in stability of hybridization duplexes formed between a probe and its corresponding target sequence comprising an allele. For example, differential probes can be designed such that under sufficiently stringent hybridization conditions, stable duplexes are formed only between the probe and its target allele sequence, but not between the probe and other allele sequences.

Probe Design

A suitable probe for instance contains a hybridizing region that is either substantially complementary or exactly complementary to a target region of a polymorphism described herein or their complement, wherein the target region encompasses the polymorphic site. The probe is typically exactly complementary to one of the two allele sequences at the polymorphic site. Suitable probes and/or hybridization conditions, which depend on the exact size and sequence of the probe, can be selected using the guidance provided herein and well known in the art. The use of oligonucleotide probes to detect nucleotide variations including single base pair differences in sequence is described in, for example, Conner et al., 1983, Proc. Natl. Acad. Sci. USA, 80:278-282, and U.S. Pat. Nos. 5,468,613 and 5,604,099, each incorporated herein by reference.

Pre-Amplification Before Probe Hybridization

In an embodiment, at least one nucleic acid sequence encompassing one or more polymorphic sites of interest are amplified or extended, and the amplified or extended product is hybridized to one or more probes under sufficiently stringent hybridization conditions. The alleles present are inferred from the pattern of binding of the probes to the amplified target sequences.

Some Known Probe-Based Genotyping Assays

Probe-based genotyping can be carried out using a “TaqMan” or “5′-nuclease assay,” as described in U.S. Pat. Nos. 5,210,015; 5,487,972; and 5,804,375; and Holland et al., 1988, Proc. Natl. Acad. Sci. USA, 88:7276-7280, each incorporated herein by reference. Examples of other techniques that can be used for SNP genotyping include, but are not limited to, Amplifluor, Dye Binding-Intercalation, Fluorescence Resonance Energy Transfer (FRET), Hybridization Signal Amplification Method (HSAM), HYB Probes, Invader/Cleavase Technology (Invader/CFLP), Molecular Beacons, Origen, DNA-Based Ramification Amplification (RAM), rolling circle amplification, Scorpions, Strand displacement amplification (SDA), oligonucleotide ligation (Nickerson et al., Proc. Natl Acad. Sci. USA, 87: 8923-8927) and/or enzymatic cleavage. Popular high-throughput SNP-detection methods also include template-directed dye-terminator incorporation (TDI) assay (Chen and Kwok, 1997, Nucleic Acids Res. 25: 347-353), the 5′-nuclease allele-specific hybridization TaqMan assay (Livak et al. 1995, Nature Genet. 9: 341-342), and the recently described allele-specific molecular beacon assay (Tyagi et al. 1998, Nature Biotech. 16: 49-53).

Assay Formats

Suitable assay formats for detecting hybrids formed between probes and target nucleic acid sequences in a sample are known in the art and include the immobilized target (dot-blot) format and immobilized probe (reverse dot-blot or line-blot) assay formats. Dot blot and reverse dot blot assay formats are described in U.S. Pat. Nos. 5,310,893; 5,451,512; 5,468,613; and 5,604,099; each incorporated herein by reference. In some embodiments multiple assays are conducted using a microfluidic format. See, e.g., Unger et al., 2000, Science 288:113-6.

Nucleic Acids Containing Polymorphisms of Interest

The invention also provides isolated nucleic acid molecules, e.g., oligonucleotides, probes and primers, comprising a portion of the genes, their complements, or variants thereof as identified herein. Preferably the variant comprises or flanks at least one of the polymorphic sites identified herein, such as variants associated with AAA.

Nucleic acids such as primers or probes can be labeled to facilitate detection. Oligonucleotides can be labeled by incorporating a label detectable by spectroscopic, photochemical, biochemical, immunochemical, radiological, radiochemical or chemical means. Useful labels include ³²P, fluorescent dyes, electron-dense reagents, enzymes, biotin, or haptens and proteins for which antisera or monoclonal antibodies are available.

2) Protein-Based or Phenotypic Detection of Polymorphism:

Where polymorphisms are associated with a particular phenotype, then individuals that contain the polymorphism can be identified by checking for the associated phenotype. For example, where a polymorphism causes an alteration in the structure, sequence, expression and/or amount of a protein or gene product, and/or size of a protein or gene product, the polymorphism can be detected by protein-based assay methods.

Techniques for Protein Analysis

Protein-based assay methods include electrophoresis (including capillary electrophoresis and one- and two-dimensional electrophoresis), chromatographic methods such as high performance liquid chromatography (HPLC), thin layer chromatography (TLC), hyperdiffusion chromatography, and mass spectrometry.

Antibodies

Where the structure and/or sequence of a protein is changed by a polymorphism of interest, one or more antibodies that selectively bind to the altered form of the protein can be used. Such antibodies can be generated and employed in detection assays such as fluid or gel precipitin reactions, immunodiffusion (single or double), immunoelectrophoresis, radioimmnunoassay (RIA), enzyme-linked immunosorbent assays (ELISAs), immunofluorescent assays, Western blotting and others.

3) Kits:

In certain embodiments, one or more oligonucleotides of the invention are provided in a kit or on an array useful for detecting the presence of a predisposing or a protective polymorphism in a nucleic acid sample of an individual whose risk for a vascular disorder such as AAA is being assessed. A useful kit can contain oligonucleotide specific for particular alleles of interest as well as instructions for their use to determine risk for a vascular disorder such as AAA, and optionally AMD as well. In some cases, the oligonucleotides can be in a form suitable for use as a probe, for example fixed to an appropriate support membrane. In other cases, the oligonucleotides can be intended for use as amplification primers for amplifying regions of the loci encompassing the polymorphic sites, as such primers are useful in the preferred embodiment of the invention. Alternatively, useful kits can contain a set of primers comprising an allele-specific primer for the specific amplification of alleles. As yet another alternative, a useful kit can contain antibodies to a protein that is altered in expression levels, structure and/or sequence when a polymorphism of interest is present within an individual. Other optional components of the kits include additional reagents used in the genotyping methods as described herein. For example, a kit additionally can contain amplification or sequencing primers which can, but need not, be sequence-specific, enzymes, substrate nucleotides, reagents for labeling and/or detecting nucleic acid and/or appropriate buffers for amplification or hybridization reactions.

4) Arrays:

The present invention also relates to an array, a support with immobilized oligonucleotides useful for practicing the present method. A useful array can contain oligonucleotide probes specific for polymorphisms identified herein. The oligonucleotides can be immobilized on a substrate, e.g., a membrane or glass. The oligonucleotides can, but need not, be labeled. The array can comprise one or more oligonucleotides used to detect the presence of one or more SNPs provided herein. In some embodiments, the array can be a micro-array.

The array can include primers or probes to determine assay the presence or absence of at least two of the SNPs listed in Tables 1A and 2A, sometimes at least three, at least four, at least five or at least six of the SNPs. In one embodiment, the array comprises probes or primers for detection of fewer than about 1000 different SNPs, often fewer than about 100 different SNPs, and sometimes fewer than about 50 different SNPs.

### VI. Follow-Up Procedures

Individuals diagnosed with a vascular disorder (or prognosed with an increased risk of a vascular disorder) using the methods described herein can be therapeutically or prophylactically treated. For example, the presence of AAA can be monitored by standard techniques, e.g., abdominal ultrasound, CT scan of abdomen and/or angiography of the aorta. Symptoms of abdominal aorta rupture include a pulsating sensation in the abdomen; pain in the abdomen or back which can radiate to groin, buttocks, or legs; abdominal rigidity; abdominal mass; anxiety; nausea and vomiting; clammy skin; rapid heart rate when rising to a standing position and/or shock.

The development, onset or progression of AAA can be monitored through periodic (e.g., monthly or yearly) evaluation, e.g., by ultrasound. If desired, the AAA can be treated surgically, for example when the AAA is predicted by the methods described herein to progress rapidly, or when the aneurysm is bigger than 5.5 cm in diameter. Sugical treatments include open repair and endovascular stent grafting.

### VII. Authorization of Treatment or Payment for Treatment

The invention also provides a healthcare method comprising paying for, authorizing payment for or authorizing the practice of the method of screening for susceptibility to AAA or for predicting the course of progression of AAA and optionally AMD in an individual, comprising screening for the presence or absence of genetic profile characterized by polymorphisms in the genome of the individual indicative of risk for AAA, wherein the genetic profile includes one or more single nucleotide polymorphisms selected from Table 2A and/or Table 2A.

According to the methods of the present invention, a third party, e.g., a hospital, clinic, a government entity, reimbursing party, insurance company (e.g., a health insurance company), HMO, third-party payor, or other entity which pays for, or reimburses medical expenses can authorize treatment, authorize payment for treatment, or authorize reimbursement of the costs of treatment. For example, the present invention relates to a healthcare method that includes authorizing the administration of, or authorizing payment or reimbursement for the administration of, an assay for determining an individual's susceptibility for AAA or for predicting the course of progression of AAA as disclosed herein. For example, the healthcare method can include authorizing the administration of, or authorizing payment or reimbursement for the administration of, an assay to determine an individual's susceptibility for development or progression of AAA that includes screening for the presence or absence of a genetic profile that includes one or more SNPs selected from Tables 1A and/or 2A.

### Incorporation by Reference

The entire disclosure of each of the patent documents and scientific articles referred to herein is incorporated by reference for all purposes.

### Equivalents

The invention can be embodied in other specific forms without departing from the spirit or essential characteristics thereof. The examples of the present invention presented below are provided only for illustrative purposes and not to limit the scope of the invention. All changes that come within the meaning and range of equivalency of the claims are intended to be embraced therein.

TABLE 1A
Statistical Analysis of Risk Predictive value of SNPs for AAA

| | | Freq (%) | Freq (%) | | | | | | | | | | |
|---|---|---|---|---|---|---|---|---|---|---|---|---|---|
| | | of Allele1 | of Allele2 | Freq (%) of | Total | Total | Freq (%) | Freq (%) | Freq (%) of | Total | Total | | |
| | | Homo- | Homo- | Heterozygotes | Freq (%) | Freq (%) | of Allele1 | of Allele2 | Heterozygotes | Freq (%) | Freq (%) | Genotype- | Frequencies |
| | | zygotes | zygotes | (Both Alleles) | of Allele1 | of Allele2 | Homozygotes | Homozygotes | (Both Alleles) | of Allele1 | of Allele2 | Likelihood | Chi Square (both |
| | | in Control | in Control | in Control | in Control | in Control | in AAA | in AAA | in AAA | in AAA | in AAA | Ratio (3 | collapsed-2 |
| Gene | SNP | Population | Population | Population | Population | Population | Population | Population | Population | Population | Population | categories) | categories) |
| ADAMTS19 | rs25816 | 61.6 | 3.6 | 34.8 | 79.0 | 21.0 | 77.5 | 5.6 | 16.9 | 85.9 | 14.1 | 0.00946534345255 | 0.06491900000000 |
| ADAMTS19 | rs6875250 | 73.3 | 2.4 | 24.3 | 85.5 | 14.5 | 86.8 | 0.0 | 13.2 | 93.4 | 6.6 | 0.01448446589719 | 0.00912300000000 |
| ADAMTS19 | rs25821 | 3.4 | 63.5 | 33.1 | 19.9 | 80.1 | 5.3 | 77.6 | 17.1 | 13.8 | 86.2 | 0.01672604032140 | 0.08437900000000 |
| ADAMTS19 | rs30300 | 1.4 | 77.0 | 21.6 | 12.2 | 87.8 | 0.0 | 89.5 | 10.5 | 5.3 | 94.7 | 0.02468924488255 | 0.01431700000000 |
| ADAMTS19 | rs10072248 | 1.4 | 76.4 | 22.3 | 12.5 | 87.5 | 0.0 | 88.2 | 11.8 | 5.9 | 94.1 | 0.03718922782105 | 0.02155100000000 |
| ADAMTS19 | rs10070537 | 1.4 | 76.4 | 22.3 | 12.5 | 87.5 | 0.0 | 88.2 | 11.8 | 5.9 | 94.1 | 0.03718922782105 | 0.02155100000000 |
| ADAMTS19 | rs30693 | 77.0 | 1.4 | 21.6 | 87.8 | 12.2 | 88.2 | 0.0 | 11.8 | 94.1 | 5.9 | 0.04812959788309 | 0.02755300000000 |
| ADAMTS2 | rs459668 | 53.7 | 7.4 | 38.9 | 73.1 | 26.9 | 64.5 | 1.3 | 34.2 | 81.6 | 18.4 | 0.03753239550718 | 0.03243500000000 |
| ADAMTS2 | rs467017 | 38.9 | 11.1 | 50.0 | 63.9 | 36.1 | 42.1 | 21.1 | 36.8 | 60.5 | 39.5 | 0.03860131271594 | 0.44837600000000 |
| ADAMTS2 | rs7704836 | 32.1 | 16.2 | 51.7 | 57.9 | 42.1 | 47.4 | 14.5 | 38.2 | 66.4 | 33.6 | 0.04417174606261 | 0.05646200000000 |
| ADAMTS2 | rs191415 | 54.1 | 7.1 | 38.9 | 73.5 | 26.5 | 64.5 | 1.3 | 34.2 | 81.6 | 18.4 | 0.04688182269948 | 0.03932900000000 |
| APBA2 | rs3751555 | 72.7 | 1.4 | 25.9 | 85.7 | 14.3 | 54.2 | 1.4 | 44.4 | 76.4 | 23.6 | 0.01064243475632 | 0.00693400000000 |
| APBA2 | rs12906440 | 72.6 | 1.4 | 26.0 | 85.6 | 14.4 | 56.6 | 2.6 | 40.8 | 77.0 | 23.0 | 0.02989885135437 | 0.00993400000000 |
| C1NH | MRD_4082/ | 0.0 | 85.9 | 14.1 | 7.0 | 93.0 | 1.3 | 96.0 | 2.7 | 2.7 | 97.3 | 0.00186726208905 | 0.04764800000000 |
| | rs28362944 | | | | | | | | | | | | |
| C1QDC1 | rs10843831 | 13.4 | 45.4 | 41.2 | 34.0 | 66.0 | 8.2 | 28.8 | 63.0 | 39.7 | 60.3 | 0.00377261318538 | 0.19454400000000 |
| C1QDC1 | rs10843824 | 13.5 | 45.3 | 41.2 | 34.1 | 65.9 | 9.2 | 30.3 | 60.5 | 39.5 | 60.5 | 0.01062627839570 | 0.21783700000000 |
| C1QDC1 | MRD_4087/ | 45.6 | 13.2 | 41.2 | 66.2 | 33.8 | 31.6 | 9.2 | 59.2 | 61.2 | 38.8 | 0.01936701069678 | 0.24536200000000 |
| | rs7299800 | | | | | | | | | | | | |
| C1QDC1 | rs10843834 | 52.4 | 8.1 | 39.5 | 72.1 | 27.9 | 39.5 | 5.3 | 55.3 | 67.1 | 32.9 | 0.04666234973813 | 0.22296000000000 |
| C1RL | rs3742088 | 0.3 | 88.5 | 11.1 | 5.9 | 94.1 | 1.3 | 73.7 | 25.0 | 13.8 | 86.2 | 0.00808197669747 | 0.00098500000000 |
| C1RL | MRD_4110/ | 0.0 | 94.9 | 5.1 | 2.5 | 97.5 | 0.0 | 85.5 | 14.5 | 7.2 | 92.8 | 0.00834927588604 | 0.00485600000000 |
| | rs61917913 | | | | | | | | | | | | |
| C1RL | rs744141 | 22.0 | 30.7 | 47.3 | 45.6 | 54.4 | 7.9 | 36.8 | 55.3 | 35.5 | 64.5 | 0.01068482329185 | 0.02533900000000 |
| C1RL | rs3742089 | 42.9 | 12.2 | 44.9 | 65.4 | 34.6 | 27.6 | 23.7 | 48.7 | 52.0 | 48.0 | 0.01146342739123 | 0.00232100000000 |
| C1S | MRD_4094/ | 0.0 | 99.7 | 0.3 | 0.2 | 99.8 | 0.0 | 96.1 | 3.9 | 2.0 | 98.0 | 0.01829605244842 | 0.00664300000000 |
| | not known | | | | | | | | | | | | |
| C2-BF(factorB) | rs2072634 | 0.0 | 97.0 | 3.0 | 1.5 | 98.5 | 0.0 | 88.2 | 11.8 | 5.9 | 94.1 | 0.00407777652745 | 0.00163300000000 |
| C3 | rs2230205 | 1.7 | 78.0 | 20.3 | 11.8 | 88.2 | 5.3 | 63.2 | 31.6 | 21.1 | 78.9 | 0.02199072856436 | 0.00317100000000 |
| C4BPA | rs1126618 | 63.5 | 2.4 | 34.1 | 80.6 | 19.4 | 85.5 | 3.9 | 10.5 | 90.8 | 9.2 | 0.00007619271481 | 0.00300300000000 |
| C4BPA | rs9943268 | 40.9 | 11.5 | 47.6 | 64.7 | 35.3 | 64.5 | 7.9 | 27.6 | 78.3 | 21.7 | 0.00104719125988 | 0.00141700000000 |
| C4BPA | rs7416639 | 11.5 | 40.9 | 47.6 | 35.3 | 64.7 | 7.9 | 64.5 | 27.6 | 21.7 | 78.3 | 0.00104719125988 | 0.00141700000000 |
| C6 | MRD_4419/ | 0.0 | 98.3 | 1.7 | 0.9 | 99.1 | 0.0 | 93.4 | 6.6 | 3.3 | 96.7 | 0.03566240123986 | 0.02025100000000 |
| | rs61734263 | | | | | | | | | | | | |
| C7 | rs1055021 | 0.0 | 82.1 | 17.9 | 9.0 | 91.0 | 0.0 | 71.1 | 28.9 | 14.5 | 85.5 | 0.03839121628247 | 0.04372700000000 |
| C8A | MRD_4048/ | 99.7 | 0.0 | 0.3 | 99.8 | 0.2 | 96.1 | 0.0 | 3.9 | 98.0 | 2.0 | 0.01845213405936 | 0.00675300000000 |
| | not known | | | | | | | | | | | | |
| C8A | MRD_4044/ | 0.0 | 99.7 | 0.3 | 0.2 | 99.8 | 0.0 | 96.1 | 3.9 | 2.0 | 98.0 | 0.01860997089668 | 0.00686500000000 |
| | not known | | | | | | | | | | | | |
| C9 | MRD_4392/ | 0.0 | 99.0 | 1.0 | 0.5 | 99.5 | 0.0 | 93.4 | 6.6 | 3.3 | 96.7 | 0.00880761892181 | 0.00300500000000 |
| | rs34882957 | | | | | | | | | | | | |
| COL19A1 | rs10755538 | 72.6 | 1.0 | 26.4 | 85.8 | 14.2 | 86.8 | 0.0 | 13.2 | 93.4 | 6.6 | 0.01801402151155 | 0.01176500000000 |
| COL19A1 | rs7757078 | 1.0 | 72.6 | 26.4 | 14.2 | 85.8 | 0.0 | 86.8 | 13.2 | 6.6 | 93.4 | 0.01801412280700 | 0.01176500000000 |
| COL19A1 | rs2502560 | 16.9 | 31.4 | 51.7 | 42.7 | 57.3 | 22.4 | 42.1 | 35.5 | 40.1 | 59.9 | 0.04051018503774 | 0.56188400000000 |
| COL19A1 | rs1340975 | 31.8 | 16.9 | 51.4 | 57.4 | 42.6 | 42.1 | 22.4 | 35.5 | 59.9 | 40.1 | 0.04619579411841 | 0.58735900000000 |
| CR1 | rs3737002 | 60.1 | 6.1 | 33.8 | 77.0 | 23.0 | 39.5 | 17.1 | 43.4 | 61.2 | 38.8 | 0.00097349458487 | 0.00007437100000 |
| CR1 | MRD_3980/ | 83.1 | 1.0 | 15.9 | 91.0 | 9.0 | 65.8 | 5.3 | 28.9 | 80.3 | 19.7 | 0.00263504051177 | 0.00016500000000 |
| | rs17259045 | | | | | | | | | | | | |
| CR1 | rs4844599 | 1.7 | 71.9 | 26.4 | 14.9 | 85.1 | 0.0 | 57.9 | 42.1 | 21.1 | 78.9 | 0.01321117975988 | 0.06687500000000 |
| CR1 | MRD_3987/ | 96.6 | 0.0 | 3.4 | 98.3 | 1.7 | 98.7 | 1.3 | 0.0 | 98.7 | 1.3 | 0.01997109922995 | 0.72415500000000 |
| | not known | | | | | | | | | | | | |
| CR1 | rs1408078 | 3.4 | 65.2 | 31.4 | 19.1 | 80.9 | 0.0 | 77.6 | 22.4 | 11.2 | 88.8 | 0.02132882977713 | 0.02208100000000 |
| CR1 | rs2274567 | 72.0 | 2.0 | 26.0 | 85.0 | 15.0 | 60.5 | 0.0 | 39.5 | 80.3 | 19.7 | 0.02430920388266 | 0.15823600000000 |
| CR1 | rs11118167 | 2.0 | 71.8 | 26.2 | 15.1 | 84.9 | 0.0 | 60.5 | 39.5 | 19.7 | 80.3 | 0.02596050369405 | 0.16870300000000 |
| CR1L | MRD_4008/ | 47.6 | 0.0 | 52.4 | 73.8 | 26.2 | 61.8 | 0.0 | 38.2 | 80.9 | 19.1 | 0.02649633636471 | 0.07019300000000 |
| | rs12729569 | | | | | | | | | | | | |
| CR3A(ITGAM) | MRD_4129/ | 43.1 | 7.1 | 49.8 | 68.0 | 32.0 | 59.2 | 14.5 | 26.3 | 72.4 | 27.6 | 0.00056903841669 | 0.29591300000000 |
| | rs3764327 | | | | | | | | | | | | |
| CR3A(ITGAM) | rs7206295 | 39.3 | 7.8 | 52.9 | 65.8 | 34.2 | 56.6 | 14.5 | 28.9 | 71.1 | 28.9 | 0.00065573322679 | 0.21670000000000 |
| CR3A(ITGAM) | rs889551 | 41.6 | 7.4 | 51.0 | 67.1 | 32.9 | 56.6 | 14.5 | 28.9 | 71.1 | 28.9 | 0.00154232355556 | 0.34714200000000 |
| CR3A(ITGAM) | MRD_4127/ | 41.6 | 7.8 | 50.7 | 66.9 | 33.1 | 56.6 | 14.5 | 28.9 | 71.1 | 28.9 | 0.00200357043428 | 0.32765100000000 |
| | rs8051304 | | | | | | | | | | | | |
| CR3A(ITGAM) | rs4561481 | 41.2 | 8.1 | 50.7 | 66.6 | 33.4 | 56.6 | 14.5 | 28.9 | 71.1 | 28.9 | 0.00217726333888 | 0.29094500000000 |
| CR3A(ITGAM) | rs3925075 | 22.6 | 23.6 | 53.7 | 49.5 | 50.5 | 34.2 | 27.6 | 38.2 | 53.3 | 46.7 | 0.03927271305208 | 0.40371900000000 |
| ENSG00000126759 | rs766775 | 13.2 | 63.5 | 23.3 | 24.8 | 75.2 | 30.3 | 63.2 | 6.6 | 33.6 | 66.4 | 0.00004708887557 | 0.02997900000000 |
| (CFP/ | | | | | | | | | | | | | |
| properdin) | | | | | | | | | | | | | |
| ENSG00000029559 | rs17013182 | 94.9 | 0.0 | 5.1 | 97.5 | 2.5 | 100.0 | 0.0 | 0.0 | 100.0 | 0.0 | 0.00807757234002 | 0.04741500000000 |
| (IBSP/ | | | | | | | | | | | | | |
| integrin-binding | | | | | | | | | | | | | |
| sialoprotein) | | | | | | | | | | | | | |
| ENSG00000148702 | rs2240878 | 49.3 | 10.1 | 40.5 | 69.6 | 30.4 | 28.9 | 11.8 | 59.2 | 58.6 | 41.4 | 0.00460878931386 | 0.00961500000000 |
| (HABP2) | | | | | | | | | | | | | |
| ENSG00000148702 | rs2000278 | 49.0 | 10.1 | 40.9 | 69.4 | 30.6 | 30.3 | 13.2 | 56.6 | 58.6 | 41.4 | 0.01210721685796 | 0.01086300000000 |
| FCGR2A | rs4657045 | 85.8 | 0.0 | 14.2 | 92.9 | 7.1 | 71.1 | 0.0 | 28.9 | 85.5 | 14.5 | 0.00383472959318 | 0.00380100000000 |
| FCGR2A | rs11580574 | 86.1 | 1.4 | 12.5 | 92.4 | 7.6 | 71.1 | 2.6 | 26.3 | 84.2 | 15.8 | 0.01207195386763 | 0.00190700000000 |
| HS3ST4 | rs4441276 | 43.2 | 7.1 | 49.7 | 68.1 | 31.9 | 52.6 | 14.5 | 32.9 | 69.1 | 30.9 | 0.01440483990883 | 0.81234500000000 |
| HS3ST4 | rs4286111 | 3.7 | 64.2 | 32.1 | 19.8 | 80.2 | 9.2 | 47.4 | 43.4 | 30.9 | 69.1 | 0.01558517778990 | 0.00307600000000 |
| HS3ST4 | rs4441274 | 64.2 | 3.7 | 32.1 | 80.2 | 19.8 | 48.0 | 8.0 | 44.0 | 70.0 | 30.0 | 0.02845981156147 | 0.00671300000000 |
| HS3ST4 | rs11645232 | 72.6 | 2.0 | 25.3 | 85.3 | 14.7 | 59.2 | 6.6 | 34.2 | 76.3 | 23.7 | 0.03432126484532 | 0.00779100000000 |
| HS3ST4 | rs6497910 | 62.8 | 2.4 | 34.8 | 80.2 | 19.8 | 51.3 | 7.9 | 40.8 | 71.7 | 28.3 | 0.04331230056211 | 0.02248000000000 |
| HS3ST4 | rs12103080 | 7.4 | 54.4 | 38.2 | 26.5 | 73.5 | 17.3 | 46.7 | 36.0 | 35.3 | 64.7 | 0.04743745664779 | 0.03233200000000 |
| IGLC1 | rs3814997 | 3.0 | 69.9 | 27.0 | 16.6 | 83.4 | 1.3 | 88.2 | 10.5 | 6.6 | 93.4 | 0.00280130597311 | 0.00184400000000 |
| IGLC1 | rs6003227 | 2.7 | 70.6 | 26.7 | 16.0 | 84.0 | 1.3 | 88.2 | 10.5 | 6.6 | 93.4 | 0.00398019668459 | 0.96303800000000 |
| ITGAX | rs11150614 | 8.1 | 41.6 | 50.3 | 33.3 | 66.7 | 13.2 | 56.6 | 30.3 | 28.3 | 71.7 | 0.00594686655221 | 0.24064700000000 |
| RFX3 | rs629275 | 1.4 | 79.7 | 18.9 | 10.8 | 89.2 | 0.0 | 92.1 | 7.9 | 3.9 | 96.1 | 0.01697148081353 | 0.00972600000000 |
| RFX3 | rs536485 | 80.1 | 1.4 | 18.6 | 89.4 | 10.6 | 92.1 | 0.0 | 7.9 | 96.1 | 3.9 | 0.01984867520194 | 0.01114500000000 |
| RFX3 | rs559746 | 1.7 | 76.7 | 21.5 | 12.5 | 87.5 | 0.0 | 88.0 | 12.0 | 6.0 | 94.0 | 0.04196820532165 | 0.02430700000000 |
| RFX3 | rs613518 | 1.7 | 77.0 | 21.3 | 12.3 | 87.7 | 0.0 | 88.2 | 11.8 | 5.9 | 94.1 | 0.04215534506808 | 0.02438000000000 |
| SDC4 | rs2251252 | 21.2 | 28.3 | 50.5 | 46.4 | 53.6 | 10.5 | 44.7 | 44.7 | 32.9 | 67.1 | 0.00984308768923 | 0.00274100000000 |
| SPOCK | rs1859346 | 53.4 | 7.1 | 39.5 | 73.1 | 26.9 | 32.9 | 9.2 | 57.9 | 61.8 | 38.2 | 0.00551913700925 | 0.00625600000000 |
| SPOCK | rs3756709 | 0.3 | 73.9 | 25.8 | 13.2 | 86.8 | 0.0 | 89.3 | 10.7 | 5.3 | 94.7 | 0.00923889170430 | 0.00711700000000 |
| SPOCK | rs2905965 | 1.0 | 78.0 | 20.9 | 11.5 | 88.5 | 3.9 | 86.8 | 9.2 | 8.6 | 91.4 | 0.01477067727539 | 0.30025900000000 |
| SPOCK | rs2905972 | 1.0 | 78.0 | 21.0 | 11.5 | 88.5 | 4.0 | 86.7 | 9.3 | 8.7 | 91.3 | 0.01570261642967 | 0.31666500000000 |
| SPOCK | rs11948133 | 8.4 | 49.7 | 41.9 | 29.4 | 70.6 | 11.8 | 32.9 | 55.3 | 39.5 | 60.5 | 0.03018103503871 | 0.01694600000000 |
| SPOCK | rs10491299 | 76.0 | 1.0 | 23.0 | 87.5 | 12.5 | 88.2 | 0.0 | 11.8 | 94.1 | 5.9 | 0.03689085563092 | 0.02155100000000 |
| SPOCK | rs12719499 | 75.7 | 0.3 | 24.0 | 87.7 | 12.3 | 77.6 | 3.9 | 18.4 | 86.8 | 13.2 | 0.04114261969929 | 0.78335900000000 |
| SPOCK | rs6873075 | 76.3 | 0.3 | 23.4 | 88.0 | 12.0 | 77.6 | 3.9 | 18.4 | 86.8 | 13.2 | 0.04535849973061 | 0.70639100000000 |
| SPOCK3 | rs10213065 | 3.1 | 61.7 | 35.3 | 20.7 | 79.3 | 10.5 | 57.9 | 31.6 | 26.3 | 73.7 | 0.04133065973186 | 0.13352600000000 |
| TLR7 | rs5935436 | 84.1 | 2.7 | 13.2 | 90.7 | 9.3 | 88.2 | 9.2 | 2.6 | 89.5 | 10.5 | 0.00118630379876 | 0.64367700000000 |
| TLR7 | rs179008 | 68.8 | 12.5 | 18.6 | 78.1 | 21.9 | 68.4 | 23.7 | 7.9 | 72.4 | 27.6 | 0.00857683918213 | 0.13218200000000 |
| TLR7 | rs179011 | 12.8 | 68.2 | 18.9 | 22.3 | 77.7 | 23.7 | 68.4 | 7.9 | 27.6 | 72.4 | 0.00890255925185 | 0.16577600000000 |
| TLR7 | rs864058 | 86.4 | 2.7 | 10.8 | 91.9 | 8.1 | 96.0 | 2.7 | 1.3 | 96.7 | 3.3 | 0.00940290534531 | 0.04168500000000 |
| TLR8 | rs5978593 | 36.5 | 31.1 | 32.4 | 52.7 | 47.3 | 51.3 | 42.1 | 6.6 | 54.6 | 45.4 | 0.00000339741571 | 0.67501700000000 |
| TLR8 | rs3764880 | 65.9 | 12.8 | 21.3 | 76.5 | 23.5 | 56.6 | 34.2 | 9.2 | 61.2 | 38.8 | 0.00004884109081 | 0.00013500000000 |
| TLR8 | rs3827469 | 74.0 | 7.4 | 18.6 | 83.3 | 16.7 | 72.4 | 18.4 | 9.2 | 77.0 | 23.0 | 0.00644317739345 | 0.07124000000000 |
| TLR8 | rs5741883 | 62.8 | 13.5 | 23.6 | 74.7 | 25.3 | 78.9 | 10.5 | 10.5 | 84.2 | 15.8 | 0.01406098400664 | 0.01311000000000 |
| TLR8 | rs1013150 | 12.2 | 68.2 | 19.6 | 22.0 | 78.0 | 14.5 | 77.6 | 7.9 | 18.4 | 81.6 | 0.03453901457576 | 0.34136100000000 |
| VTN | rs2227728 | 0.3 | 82.1 | 17.6 | 9.1 | 90.9 | 1.3 | 94.7 | 3.9 | 3.3 | 96.7 | 0.00250170644553 | 0.01761100000000 |
| VTN | MRD_4187/ | 0.3 | 82.1 | 17.6 | 9.1 | 90.9 | 1.3 | 94.7 | 3.9 | 3.3 | 96.7 | 0.00250170644553 | 0.01761100000000 |
| | rs2227718 | | | | | | | | | | | | |

TABLE 1B
Raw Data for SNP Genotypes in Control and AAA Population

| | | | | Number | Number | Number of | | | | | |
|---|---|---|---|---|---|---|---|---|---|---|---|
| | | Number with. | | of Allele1 | of Allele2 | Heterozygotes | Number with. | | | | Number of |
| | | Undetermined- | Size of | Homozygotes | Homozygotes | (Both Alleles) | Undetermined- | | Number of Allele1 | Number of Allele2 | Heterozygotes |
| | | Genotype in | Control | in Control | in Control | in Control | Genotype | Size of AAA | Homozygotes | Homozygotes | (Both Alleles) |
| Gene | SNP | Control Popn. | Population | Population | Population | Population | in AAA Popn. | Population | in AAA Population | in AAA Population | in AAA Population |
| ADAMTS19 | rs25816 | 17 | 279 | 172 | 10 | 97 | 5 | 71 | 55 | 4 | 12 |
| ADAMTS19 | rs6875250 | 0 | 296 | 217 | 7 | 72 | 0 | 76 | 66 | 0 | 10 |
| ADAMTS19 | rs25821 | 0 | 296 | 10 | 188 | 98 | 0 | 76 | 4 | 59 | 13 |
| ADAMTS19 | rs30300 | 0 | 296 | 4 | 228 | 64 | 0 | 76 | 0 | 68 | 8 |
| ADAMTS19 | rs10072248 | 0 | 296 | 4 | 226 | 66 | 0 | 76 | 0 | 67 | 9 |
| ADAMTS19 | rs10070537 | 0 | 296 | 4 | 226 | 66 | 0 | 76 | 0 | 67 | 9 |
| ADAMTS19 | rs30693 | 0 | 296 | 228 | 4 | 64 | 0 | 76 | 67 | 0 | 9 |
| ADAMTS2 | rs459668 | 0 | 296 | 159 | 22 | 115 | 0 | 76 | 49 | 1 | 26 |
| ADAMTS2 | rs467017 | 0 | 296 | 115 | 33 | 148 | 0 | 76 | 32 | 16 | 28 |
| ADAMTS2 | rs7704836 | 0 | 296 | 95 | 48 | 153 | 0 | 76 | 36 | 11 | 29 |
| ADAMTS2 | rs191415 | 0 | 296 | 160 | 21 | 115 | 0 | 76 | 49 | 1 | 26 |
| APBA2 | rs3751555 | 10 | 286 | 208 | 4 | 74 | 4 | 72 | 39 | 1 | 32 |
| APBA2 | rs12906440 | 4 | 292 | 212 | 4 | 76 | 0 | 76 | 43 | 2 | 31 |
| C1NH | MRD_4082/ | 26 | 270 | 0 | 232 | 38 | 1 | 75 | 1 | 72 | 2 |
| | rs28362944 | | | | | | | | | | |
| C1QDC1 | rs10843831 | 12 | 284 | 38 | 129 | 117 | 3 | 73 | 6 | 21 | 46 |
| C1QDC1 | rs10843824 | 0 | 296 | 40 | 134 | 122 | 0 | 76 | 7 | 23 | 46 |
| C1QDC1 | MRD_4087/ | 0 | 296 | 135 | 39 | 122 | 0 | 76 | 24 | 7 | 45 |
| | rs7299800 | | | | | | | | | | |
| C1QDC1 | rs10843834 | 0 | 296 | 155 | 24 | 117 | 0 | 76 | 30 | 4 | 42 |
| C1RL | rs3742088 | 0 | 296 | 1 | 262 | 33 | 0 | 76 | 1 | 56 | 19 |
| C1RL | MRD_4110/ | 0 | 296 | 0 | 281 | 15 | 0 | 76 | 0 | 65 | 11 |
| | rs61917913 | | | | | | | | | | |
| C1RL | rs744141 | 0 | 296 | 65 | 91 | 140 | 0 | 76 | 6 | 28 | 42 |
| C1RL | rs3742089 | 0 | 296 | 127 | 36 | 133 | 0 | 76 | 21 | 18 | 37 |
| C1S | MRD_4094/ | 0 | 296 | 0 | 295 | 1 | 0 | 76 | 0 | 73 | 3 |
| | not known | | | | | | | | | | |
| C2- | rs2072634 | 0 | 296 | 0 | 287 | 9 | 0 | 76 | 0 | 67 | 9 |
| BF(factorB) | | | | | | | | | | | |
| C3 | rs2230205 | 0 | 296 | 5 | 231 | 60 | 0 | 76 | 4 | 48 | 24 |
| C4BPA | rs1126618 | 0 | 296 | 188 | 7 | 101 | 0 | 76 | 65 | 3 | 8 |
| C4BPA | rs9943268 | 0 | 296 | 121 | 34 | 141 | 0 | 76 | 49 | 6 | 21 |
| C4BPA | rs7416639 | 0 | 296 | 34 | 121 | 141 | 0 | 76 | 6 | 49 | 21 |
| C6 | MRD_4419/ | 2 | 294 | 0 | 289 | 5 | 0 | 76 | 0 | 71 | 5 |
| | rs61734263 | | | | | | | | | | |
| C7 | rs1055021 | 0 | 296 | 0 | 243 | 53 | 0 | 76 | 0 | 54 | 22 |
| C8A | MRD_4048/ | 1 | 295 | 294 | 0 | 1 | 0 | 76 | 73 | 0 | 3 |
| | not known | | | | | | | | | | |
| C8A | MRD_4044/ | 2 | 294 | 0 | 293 | 1 | 0 | 76 | 0 | 73 | 3 |
| | not known | | | | | | | | | | |
| C9 | MRD_4392/ | 0 | 296 | 0 | 293 | 3 | 0 | 76 | 0 | 71 | 5 |
| | rs34882957 | | | | | | | | | | |
| COL19A1 | rs10755538 | 0 | 296 | 215 | 3 | 78 | 0 | 76 | 66 | 0 | 10 |
| COL19A1 | rs7757078 | 0 | 296 | 3 | 215 | 78 | 0 | 76 | 0 | 66 | 10 |
| COL19A1 | rs2502560 | 0 | 296 | 50 | 93 | 153 | 0 | 76 | 17 | 32 | 27 |
| COL19A1 | rs1340975 | 0 | 296 | 94 | 50 | 152 | 0 | 76 | 32 | 17 | 27 |
| CR1 | rs3737002 | 0 | 296 | 178 | 18 | 100 | 0 | 76 | 30 | 13 | 33 |
| CR1 | MRD_3980/ | 0 | 296 | 246 | 3 | 47 | 0 | 76 | 50 | 4 | 22 |
| | rs17259045 | | | | | | | | | | |
| CR1 | rs4844599 | 1 | 295 | 5 | 212 | 78 | 0 | 76 | 0 | 44 | 32 |
| CR1 | MRD_3987/ | 6 | 290 | 280 | 0 | 10 | 0 | 76 | 75 | 1 | 0 |
| | not known | | | | | | | | | | |
| CR1 | rs1408078 | 0 | 296 | 10 | 193 | 93 | 0 | 76 | 0 | 59 | 17 |
| CR1 | rs2274567 | 0 | 296 | 213 | 6 | 77 | 0 | 76 | 46 | 0 | 30 |
| CR1 | rs11118167 | 2 | 294 | 6 | 211 | 77 | 0 | 76 | 0 | 46 | 30 |
| CR1L | MRD_4008/ | 0 | 296 | 141 | 0 | 155 | 0 | 76 | 47 | 0 | 29 |
| | rs12729569 | | | | | | | | | | |
| CR3A(ITGAM) | MRD_4129/ | 1 | 295 | 127 | 21 | 147 | 0 | 76 | 45 | 11 | 20 |
| | rs3764327 | | | | | | | | | | |
| CR3A(ITGAM) | rs7206295 | 1 | 295 | 116 | 23 | 156 | 0 | 76 | 43 | 11 | 22 |
| CR3A(ITGAM) | rs889551 | 0 | 296 | 123 | 22 | 151 | 0 | 76 | 43 | 11 | 22 |
| CR3A(ITGAM) | MRD_4127/ | 0 | 296 | 123 | 23 | 150 | 0 | 76 | 43 | 11 | 22 |
| | rs8051304 | | | | | | | | | | |
| CR3A(ITGAM) | rs4561481 | 0 | 296 | 122 | 24 | 150 | 0 | 76 | 43 | 11 | 22 |
| CR3A(ITGAM) | rs3925075 | 0 | 296 | 67 | 70 | 159 | 0 | 76 | 26 | 21 | 29 |
| ENSG00000126759 | rs766775 | 0 | 296 | 39 | 188 | 69 | 0 | 76 | 23 | 48 | 5 |
| (CFP/ | | | | | | | | | | | |
| properdin) | | | | | | | | | | | |
| ENSG00000029559 | rs17013182 | 0 | 296 | 281 | 0 | 15 | 0 | 76 | 76 | 0 | 0 |
| (IBSP/ | | | | | | | | | | | |
| integrin- | | | | | | | | | | | |
| binding | | | | | | | | | | | |
| sialoprotein) | | | | | | | | | | | |
| ENSG00000148702 | rs2240878 | 0 | 296 | 146 | 30 | 120 | 0 | 76 | 22 | 9 | 45 |
| (HABP2) | | | | | | | | | | | |
| ENSG00000148702 | rs2000278 | 0 | 296 | 145 | 30 | 121 | 0 | 76 | 23 | 10 | 43 |
| FCGR2A | rs4657045 | 0 | 296 | 254 | 0 | 42 | 0 | 76 | 54 | 0 | 22 |
| FCGR2A | rs11580574 | 0 | 296 | 255 | 4 | 37 | 0 | 76 | 54 | 2 | 20 |
| HS3ST4 | rs4441276 | 0 | 296 | 128 | 21 | 147 | 0 | 76 | 40 | 11 | 25 |
| HS3ST4 | rs4286111 | 0 | 296 | 11 | 190 | 95 | 0 | 76 | 7 | 36 | 33 |
| HS3ST4 | rs4441274 | 0 | 296 | 190 | 11 | 95 | 1 | 75 | 36 | 6 | 33 |
| HS3ST4 | rs11645232 | 0 | 296 | 215 | 6 | 75 | 0 | 76 | 45 | 5 | 26 |
| HS3ST4 | rs6497910 | 0 | 296 | 186 | 7 | 103 | 0 | 76 | 39 | 6 | 31 |
| HS3ST4 | rs12103080 | 0 | 296 | 22 | 161 | 113 | 1 | 75 | 13 | 35 | 27 |
| IGLC1 | rs3814997 | 0 | 296 | 9 | 207 | 80 | 0 | 76 | 1 | 67 | 8 |
| IGLC1 | rs6003227 | 0 | 296 | 8 | 209 | 79 | 0 | 76 | 1 | 67 | 8 |
| ITGAX | rs11150614 | 0 | 296 | 24 | 123 | 149 | 0 | 76 | 10 | 43 | 23 |
| RFX3 | rs629275 | 0 | 296 | 4 | 236 | 56 | 0 | 76 | 0 | 70 | 6 |
| RFX3 | rs536485 | 0 | 296 | 237 | 4 | 55 | 0 | 76 | 70 | 0 | 6 |
| RFX3 | rs559746 | 8 | 288 | 5 | 221 | 62 | 1 | 75 | 0 | 66 | 9 |
| RFX3 | rs613518 | 0 | 296 | 5 | 228 | 63 | 0 | 76 | 0 | 67 | 9 |
| SDC4 | rs2251252 | 3 | 293 | 62 | 83 | 148 | 0 | 76 | 8 | 34 | 34 |
| SPOCK | rs1859346 | 0 | 296 | 158 | 21 | 117 | 0 | 76 | 25 | 7 | 44 |
| SPOCK | rs3756709 | 1 | 295 | 1 | 218 | 76 | 1 | 75 | 0 | 67 | 8 |
| SPOCK | rs2905965 | 0 | 296 | 3 | 231 | 62 | 0 | 76 | 3 | 66 | 7 |
| SPOCK | rs2905972 | 1 | 295 | 3 | 230 | 62 | 1 | 75 | 3 | 65 | 7 |
| SPOCK | rs11948133 | 0 | 296 | 25 | 147 | 124 | 0 | 76 | 9 | 25 | 42 |
| SPOCK | rs10491299 | 0 | 296 | 225 | 3 | 68 | 0 | 76 | 67 | 0 | 9 |
| SPOCK | rs12719499 | 0 | 296 | 224 | 1 | 71 | 0 | 76 | 59 | 3 | 14 |
| SPOCK | rs6873075 | 1 | 295 | 225 | 1 | 69 | 0 | 76 | 59 | 3 | 14 |
| SPOCK3 | rs10213065 | 1 | 295 | 9 | 182 | 104 | 0 | 76 | 8 | 44 | 24 |
| TLR7 | rs5935436 | 0 | 296 | 249 | 8 | 39 | 0 | 76 | 67 | 7 | 2 |
| TLR7 | rs179008 | 1 | 295 | 203 | 37 | 55 | 0 | 76 | 52 | 18 | 6 |
| TLR7 | rs179011 | 0 | 296 | 38 | 202 | 56 | 0 | 76 | 18 | 52 | 6 |
| TLR7 | rs864058 | 1 | 295 | 255 | 8 | 32 | 1 | 75 | 72 | 2 | 1 |
| TLR8 | rs5978593 | 0 | 296 | 108 | 92 | 96 | 0 | 76 | 39 | 32 | 5 |
| TLR8 | rs3764880 | 0 | 296 | 195 | 38 | 63 | 0 | 76 | 43 | 26 | 7 |
| TLR8 | rs3827469 | 0 | 296 | 219 | 22 | 55 | 0 | 76 | 55 | 14 | 7 |
| TLR8 | rs5741883 | 0 | 296 | 186 | 40 | 70 | 0 | 76 | 60 | 8 | 8 |
| TLR8 | rs1013150 | 0 | 296 | 36 | 202 | 58 | 0 | 76 | 11 | 59 | 6 |
| VTN | rs2227728 | 0 | 296 | 1 | 243 | 52 | 0 | 76 | 1 | 72 | 3 |
| VTN | MRD_4187/ | 0 | 296 | 1 | 243 | 52 | 0 | 76 | 1 | 72 | 3 |
| | rs2227718 | | | | | | | | | | |

TABLE 2A
Statistical Analysis of Risk-Predictive Value of SNPs fpr AAA + AMD

| | | Freq (%) | Freq (%) | | | | | | | | | | |
|---|---|---|---|---|---|---|---|---|---|---|---|---|---|
| | | of Allele1 | of Allele2 | Freq (%) of | Total | Total | Freq (%) | Freq (%) | Freq (%) of | Total | Total | | |
| | | Homo- | Homo- | Heterozygotes | Freq (%) | Freq (%) | of Allele1 | of Allele2 | Heterozygotes | Freq (%) | Freq (%) | Genotype- | Frequencies |
| | | zygotes | zygotes | (Both Alleles) | of Allele1 | of Allele2 | Homozygotes | Homozygotes | (Both Alleles) | of Allele1 | of Allele2 | Likelihood | Chi Square (both |
| | | in Control | in Control | in Control | in Control | in Control | in AAA | in AAA | in AAA | in AAA | in AAA | Ratio (3 | collapsed-2 |
| Gene | SNP | Population | Population | Population | Population | Population | Population | Population | Population | Population | Population | categories) | categories) |
| ADAM12 | rs4962543 | 47.6 | 14.9 | 37.5 | 66.4 | 33.6 | 55.7 | 1.4 | 42.9 | 77.1 | 22.9 | 0.00115032440099 | 0.01377500000000 |
| ADAM12 | rs1621212 | 29.7 | 17.2 | 53.0 | 56.3 | 43.8 | 52.9 | 12.9 | 34.3 | 70.0 | 30.0 | 0.00149370551840 | 0.00294500000000 |
| ADAM12 | rs1676717 | 17.6 | 29.0 | 53.4 | 44.3 | 55.7 | 13.0 | 52.2 | 34.8 | 30.4 | 69.6 | 0.00151887511182 | 0.00296000000000 |
| ADAM12 | rs12779767 | 41.9 | 10.8 | 47.3 | 65.5 | 34.5 | 30.0 | 25.7 | 44.3 | 52.1 | 47.9 | 0.00651015586679 | 0.00315300000000 |
| ADAM12 | rs11244834 | 10.8 | 41.4 | 47.8 | 34.7 | 65.3 | 25.7 | 30.0 | 44.3 | 47.9 | 52.1 | 0.00717120449836 | 0.00391900000000 |
| ADAM12 | rs1674923 | 15.2 | 38.2 | 46.6 | 38.5 | 61.5 | 27.1 | 24.3 | 48.6 | 51.4 | 48.6 | 0.02313154308755 | 0.00520100000000 |
| ADAM12 | rs1676736 | 38.2 | 15.2 | 46.6 | 61.5 | 38.5 | 24.3 | 27.1 | 48.6 | 48.6 | 51.4 | 0.02313154308756 | 0.00520100000000 |
| ADAM12 | rs1674888 | 33.4 | 17.9 | 48.6 | 57.8 | 42.2 | 27.1 | 8.6 | 64.3 | 59.3 | 40.7 | 0.03313334657090 | 0.74383400000000 |
| ADRB2 | rs1800888 | 98.3 | 0.3 | 1.4 | 99.0 | 1.0 | 90.0 | 0.0 | 10.0 | 95.0 | 5.0 | 0.00356751745165 | 0.00132000000000 |
| APOD | rs2280520 | 2.4 | 69.3 | 28.4 | 16.6 | 83.4 | 4.3 | 85.7 | 10.0 | 9.3 | 90.7 | 0.00227636958690 | 0.03106100000000 |
| APOD | rs4677695 | 2.4 | 69.9 | 27.7 | 16.2 | 83.8 | 4.3 | 85.7 | 10.0 | 9.3 | 90.7 | 0.00322805474437 | 0.03831100000000 |
| APOD | rs4677692 | 69.9 | 2.4 | 27.7 | 83.8 | 16.2 | 85.7 | 4.3 | 10.0 | 90.7 | 9.3 | 0.00322805474437 | 0.03831100000000 |
| BMP7 | rs6064517 | 83.8 | 1.0 | 15.2 | 91.4 | 8.6 | 64.3 | 1.4 | 34.3 | 81.4 | 18.6 | 0.00217067777749 | 0.00055400000000 |
| BMP7 | rs6014959 | 83.4 | 1.4 | 15.3 | 91.0 | 9.0 | 64.3 | 1.4 | 34.3 | 81.4 | 18.6 | 0.00247335646260 | 0.00102600000000 |
| BMP7 | rs6064506 | 28.7 | 27.7 | 43.6 | 50.5 | 49.5 | 20.0 | 15.7 | 64.3 | 52.1 | 47.9 | 0.00664365904290 | 0.72766700000000 |
| BMP7 | rs6025422 | 29.7 | 26.4 | 43.9 | 51.7 | 48.3 | 24.3 | 12.9 | 62.9 | 55.7 | 44.3 | 0.00823247897107 | 0.39109500000000 |
| BMP7 | rs6127984 | 14.2 | 40.2 | 45.6 | 37.0 | 63.0 | 4.3 | 47.1 | 48.6 | 28.6 | 71.4 | 0.03967795481190 | 0.06090900000000 |
| BMP7 | rs8116259 | 40.2 | 14.2 | 45.6 | 63.0 | 37.0 | 47.1 | 4.3 | 48.6 | 71.4 | 28.6 | 0.03967795481190 | 0.06090900000000 |
| BMP7 | rs162315 | 5.1 | 64.5 | 30.4 | 20.3 | 79.7 | 5.7 | 48.6 | 45.7 | 28.6 | 71.4 | 0.04513630722508 | 0.03257500000000 |
| BMP7 | rs162316 | 5.1 | 64.5 | 30.4 | 20.3 | 79.7 | 5.7 | 48.6 | 45.7 | 28.6 | 71.4 | 0.04513630722508 | 0.03257500000000 |
| C1NH | MRD_4082/ | 0.0 | 85.9 | 14.1 | 7.0 | 93.0 | 0.0 | 96.9 | 3.1 | 1.5 | 98.5 | 0.00530126568380 | 0.01752500000000 |
| | rs28362944 | | | | | | | | | | | | |
| C1QTNF7 | rs4235376 | 0.7 | 88.8 | 10.5 | 5.9 | 94.1 | 0.0 | 74.3 | 25.7 | 12.9 | 87.1 | 0.00523301586261 | 0.00452800000000 |
| C1QTNF7 | rs13116208 | 8.5 | 46.8 | 44.7 | 30.8 | 69.2 | 21.4 | 42.9 | 35.7 | 39.3 | 60.7 | 0.01370839455219 | 0.05524700000000 |
| C1QTNF7 | rs16891811 | 2.1 | 75.5 | 22.4 | 13.3 | 86.7 | 8.7 | 62.3 | 29.0 | 23.2 | 76.8 | 0.01772693843616 | 0.00353900000000 |
| C1QTNF7 | rs4698382 | 79.7 | 2.0 | 18.2 | 88.9 | 11.1 | 68.6 | 0.0 | 31.4 | 84.3 | 15.7 | 0.02089298500683 | 0.13522300000000 |
| C1QTNF7 | rs4505816 | 75.0 | 2.4 | 22.6 | 86.3 | 13.7 | 61.4 | 8.6 | 30.0 | 76.4 | 23.6 | 0.02255862743859 | 0.00370900000000 |
| C1QTNF7 | rs2192356 | 7.9 | 47.4 | 44.7 | 30.2 | 69.8 | 18.8 | 43.5 | 37.7 | 37.7 | 62.3 | 0.03898838625621 | 0.09114800000000 |
| C1QTNF7 | rs2215809 | 3.4 | 75.3 | 21.3 | 14.0 | 86.0 | 2.9 | 61.4 | 35.7 | 20.7 | 79.3 | 0.04877020626574 | 0.04785900000000 |
| C1RL | MRD_4110/ | 0.0 | 94.9 | 5.1 | 2.5 | 97.5 | 2.9 | 90.0 | 7.1 | 6.4 | 93.6 | 0.02758602381634 | 0.01995200000000 |
| | rs61917913 | | | | | | | | | | | | |
| C2-BF | rs4151670 | 92.9 | 0.0 | 7.1 | 96.5 | 3.5 | 98.6 | 0.0 | 1.4 | 99.3 | 0.7 | 0.03858866067236 | 0.07746600000000 |
| (factorB) | | | | | | | | | | | | | |
| C3 | MRD_4273/ | 6.7 | 52.4 | 40.9 | 27.1 | 72.9 | 1.5 | 66.7 | 31.8 | 17.4 | 82.6 | 0.04191430933243 | 0.02130100000000 |
| | rs2547438 | | | | | | | | | | | | |
| C3AR1 | rs10846411 | 12.5 | 44.4 | 43.1 | 34.1 | 65.9 | 2.9 | 44.3 | 52.9 | 29.3 | 70.7 | 0.02079661846701 | 0.27989100000000 |
| C5 | rs10116271 | 23.6 | 32.1 | 44.3 | 45.8 | 54.2 | 22.9 | 17.1 | 60.0 | 52.9 | 47.1 | 0.02100530416723 | 0.13124000000000 |
| C6 | rs10071904 | 2.4 | 67.6 | 30.1 | 17.4 | 82.6 | 5.7 | 78.6 | 15.7 | 13.6 | 86.4 | 0.02224816205693 | 0.27450600000000 |
| C6 | rs6892389 | 73.0 | 2.0 | 25.0 | 85.5 | 14.5 | 81.4 | 5.7 | 12.9 | 87.9 | 12.1 | 0.02921877440089 | 0.46564600000000 |
| C6 | rs2910644 | 79.1 | 2.7 | 18.2 | 88.2 | 11.8 | 64.3 | 2.9 | 32.9 | 80.7 | 19.3 | 0.03323790726041 | 0.01919700000000 |
| C6 | MRD_4420/ | 0.7 | 94.6 | 4.7 | 3.0 | 97.0 | 0.0 | 87.1 | 12.9 | 6.4 | 93.6 | 0.04685696181743 | 0.05578400000000 |
| | rs61733159 | | | | | | | | | | | | |
| C7 | rs1055021 | 0.0 | 82.1 | 17.9 | 9.0 | 91.0 | 4.3 | 78.6 | 17.1 | 12.9 | 87.1 | 0.00663562701388 | 0.16037500000000 |
| C7 | rs2271708 | 99.7 | 0.0 | 0.3 | 99.8 | 0.2 | 95.7 | 0.0 | 4.3 | 97.9 | 2.1 | 0.01479556366548 | 0.00438300000000 |
| CLUL1 | rs10502288 | 8.1 | 56.4 | 35.5 | 25.8 | 74.2 | 5.7 | 37.1 | 57.1 | 34.3 | 65.7 | 0.00429471460876 | 0.04416800000000 |
| CLUL1 | rs8093432 | 65.2 | 3.7 | 31.1 | 80.7 | 19.3 | 48.6 | 5.7 | 45.7 | 71.4 | 28.6 | 0.03881295483255 | 0.01502500000000 |
| COL12A1 | rs4708174 | 2.7 | 71.3 | 26.0 | 15.7 | 84.3 | 0.0 | 82.9 | 17.1 | 8.6 | 91.4 | 0.04090976949578 | 0.03024600000000 |
| COL19A1 | rs10485243 | 64.3 | 4.1 | 31.6 | 80.1 | 19.9 | 49.3 | 11.6 | 39.1 | 68.8 | 31.2 | 0.02045203598462 | 0.00407600000000 |
| COL19A1 | rs737330 | 62.5 | 4.1 | 33.4 | 79.2 | 20.8 | 48.6 | 11.4 | 40.0 | 68.6 | 31.4 | 0.02665684333072 | 0.00691500000000 |
| COL19A1 | rs2145905 | 0.8 | 72.6 | 26.6 | 14.1 | 85.9 | 6.3 | 66.7 | 27.0 | 19.8 | 80.2 | 0.03632897656497 | 0.10483600000000 |
| CR3 | rs235328 | 56.9 | 8.8 | 34.2 | 74.1 | 25.9 | 57.1 | 1.4 | 41.4 | 77.9 | 22.1 | 0.03667975146539 | 0.35322700000000 |
| ENSG00000000971 | rs10801554 | 15.2 | 39.5 | 45.3 | 37.8 | 62.2 | 28.6 | 27.1 | 44.3 | 50.7 | 49.3 | 0.02275934557795 | 0.00521700000000 |
| (CFH) | | | | | | | | | | | | | |
| ENSG00000000971 | rs1329421 | 39.5 | 15.2 | 45.3 | 62.2 | 37.8 | 27.1 | 28.6 | 44.3 | 49.3 | 50.7 | 0.02275934557795 | 0.00521700000000 |
| ENSG00000126759 | rs766775 | 13.2 | 63.5 | 23.3 | 24.8 | 75.2 | 21.4 | 70.0 | 8.6 | 25.7 | 74.3 | 0.00654155600933 | 0.82818100000000 |
| (properdin) | | | | | | | | | | | | | |
| ENSG00000148702 | rs11575688 | 0.0 | 96.9 | 3.1 | 1.5 | 98.5 | 1.4 | 88.4 | 10.1 | 6.5 | 93.5 | 0.01168833521131 | 0.00066700000000 |
| (HABP2) | | | | | | | | | | | | | |
| ENSG00000148702 | rs7080536 | 0.0 | 95.2 | 4.8 | 2.4 | 97.6 | 0.0 | 85.3 | 14.7 | 7.4 | 92.6 | 0.00702829644318 | 0.00351400000000 |
| ENSG00000197467 | rs3108966 | 45.3 | 9.8 | 44.9 | 67.7 | 32.3 | 61.4 | 4.3 | 34.3 | 78.6 | 21.4 | 0.03372986330004 | 0.01202800000000 |
| ENSG00000197467 | rs3104052 | 45.4 | 9.8 | 44.7 | 67.8 | 32.2 | 61.4 | 4.3 | 34.3 | 78.6 | 21.4 | 0.03532204127310 | 0.01249500000000 |
| FBLN2 | rs4684148 | 10.2 | 47.1 | 42.7 | 31.5 | 68.5 | 4.3 | 65.2 | 30.4 | 19.6 | 80.4 | 0.01731212405203 | 0.00543200000000 |
| FBN2 | rs331079 | 1.0 | 80.1 | 18.9 | 10.5 | 89.5 | 0.0 | 92.9 | 7.1 | 3.6 | 96.4 | 0.01754876925976 | 0.01087500000000 |
| FBN2 | rs10073062 | 49.7 | 5.7 | 44.6 | 72.0 | 28.0 | 57.1 | 12.9 | 30.0 | 72.1 | 27.9 | 0.02756653539265 | 0.96533500000000 |
| FBN2 | rs27913 | 67.2 | 3.0 | 29.7 | 82.1 | 17.9 | 72.9 | 8.6 | 18.6 | 82.1 | 17.9 | 0.03778259847953 | 0.98931100000000 |
| FBN2 | rs468182 | 67.2 | 3.0 | 29.7 | 82.1 | 17.9 | 72.9 | 8.6 | 18.6 | 82.1 | 17.9 | 0.03778259847953 | 0.98931100000000 |
| FCGR2A | rs4657045 | 85.8 | 0.0 | 14.2 | 92.9 | 7.1 | 71.4 | 0.0 | 28.6 | 85.7 | 14.3 | 0.00620559921940 | 0.00599300000000 |
| FCGR2A | rs11580574 | 86.1 | 1.4 | 12.5 | 92.4 | 7.6 | 71.4 | 2.9 | 25.7 | 84.3 | 15.7 | 0.01907758419599 | 0.00275600000000 |
| FCN1 | rs1071583 | 37.8 | 17.2 | 44.9 | 60.3 | 39.7 | 54.3 | 7.1 | 38.6 | 73.6 | 26.4 | 0.01420147783817 | 0.00348400000000 |
| FCN1 | rs2989727 | 17.9 | 35.8 | 46.3 | 41.0 | 59.0 | 8.6 | 50.0 | 41.4 | 29.3 | 70.7 | 0.03652975053835 | 0.01021900000000 |
| FCN2 | rs3124953 | 5.4 | 58.5 | 36.1 | 23.5 | 76.5 | 13.0 | 68.1 | 18.8 | 22.5 | 77.5 | 0.00509527337794 | 0.28821500000000 |
| FHR5 | MRD_3914/ | 100.0 | 0.0 | 0.0 | 100.0 | 0.0 | 95.5 | 0.0 | 4.5 | 97.8 | 2.2 | 0.00136701139762 | 0.00026400000000 |
| | rs41306229 | | | | | | | | | | | | |
| FHR5 | MRD_3905/ | 3.0 | 57.8 | 39.2 | 22.6 | 77.4 | 8.6 | 68.6 | 22.9 | 20.0 | 80.0 | 0.00994191724806 | 0.49939500000000 |
| | not known | | | | | | | | | | | | |
| FHR5 | rs3748557 | 57.8 | 4.1 | 38.2 | 76.9 | 23.1 | 67.1 | 10.0 | 22.9 | 78.6 | 21.4 | 0.01623526582267 | 0.66396400000000 |
| FHR5 | MRD_3906/ | 57.8 | 3.7 | 38.5 | 77.0 | 23.0 | 68.6 | 8.6 | 22.9 | 80.0 | 20.0 | 0.01965380073064 | 0.44802700000000 |
| | not known | | | | | | | | | | | | |
| HS3ST4 | rs9938946 | 75.7 | 3.0 | 21.3 | 86.3 | 13.7 | 64.3 | 0.0 | 35.7 | 82.1 | 17.9 | 0.00968050279783 | 0.20683700000000 |
| HS3ST4 | rs4441276 | 43.2 | 7.1 | 49.7 | 68.1 | 31.9 | 37.1 | 20.0 | 42.9 | 58.6 | 41.4 | 0.01007177526315 | 0.03247300000000 |
| HS3ST4 | rs7197707 | 19.9 | 31.4 | 48.6 | 44.3 | 55.7 | 7.1 | 41.4 | 51.4 | 32.9 | 67.1 | 0.01663171601116 | 0.01400900000000 |
| HS3ST4 | rs3923426 | 78.7 | 2.7 | 18.6 | 88.0 | 12.0 | 68.6 | 0.0 | 31.4 | 84.3 | 15.7 | 0.01677409805582 | 0.23447100000000 |
| HS3ST4 | rs9928833 | 4.4 | 68.9 | 26.7 | 17.7 | 82.3 | 0.0 | 62.9 | 37.1 | 18.6 | 81.4 | 0.01975658669630 | 0.81671400000000 |
| HS3ST4 | rs4286111 | 3.7 | 64.2 | 32.1 | 19.8 | 80.2 | 7.2 | 47.8 | 44.9 | 29.7 | 70.3 | 0.03893698393991 | 0.01061500000000 |
| HS3ST4 | rs11074715 | 38.2 | 12.5 | 49.3 | 62.8 | 37.2 | 50.0 | 4.3 | 45.7 | 72.9 | 27.1 | 0.04223152020324 | 0.02564100000000 |
| HS3ST4 | rs4287571 | 12.2 | 39.2 | 48.6 | 36.5 | 63.5 | 4.3 | 51.4 | 44.3 | 26.4 | 73.6 | 0.04465332394615 | 0.02442300000000 |
| HS3ST4 | rs8044250 | 73.4 | 3.8 | 22.8 | 84.8 | 15.2 | 82.6 | 0.0 | 17.4 | 91.3 | 8.7 | 0.04661658407932 | 0.04680300000000 |
| HS3ST4 | rs4441274 | 64.2 | 3.7 | 32.1 | 80.2 | 19.8 | 48.6 | 7.1 | 44.3 | 70.7 | 29.3 | 0.04939745838776 | 0.01378500000000 |
| HS3ST4 | rs4523929 | 50.0 | 12.8 | 37.2 | 68.6 | 31.4 | 34.3 | 14.3 | 51.4 | 60.0 | 40.0 | 0.04959700832089 | 0.05226300000000 |
| ITGA6 | rs12471315 | 9.1 | 48.3 | 42.6 | 30.4 | 69.6 | 0.0 | 58.0 | 42.0 | 21.0 | 79.0 | 0.00211022708462 | 0.02796700000000 |
| ITGA6 | rs12464480 | 7.5 | 51.4 | 41.2 | 28.1 | 71.9 | 0.0 | 61.4 | 38.6 | 19.3 | 80.7 | 0.00517415713203 | 0.03420300000000 |
| ITGA6 | rs10497383 | 69.3 | 4.7 | 26.0 | 82.3 | 17.7 | 75.7 | 0.0 | 24.3 | 87.9 | 12.1 | 0.04202005324775 | 0.11024600000000 |
| ITGA6 | rs10497384 | 4.7 | 69.3 | 26.0 | 17.7 | 82.3 | 0.0 | 75.7 | 24.3 | 12.1 | 87.9 | 0.04202043702854 | 0.11024600000000 |
| ITGA6 | rs1076594 | 42.7 | 10.2 | 47.1 | 66.3 | 33.7 | 54.3 | 2.9 | 42.9 | 75.7 | 24.3 | 0.04391850899102 | 0.03117600000000 |
| MASP1 | rs698086 | 12.2 | 39.9 | 48.0 | 36.1 | 63.9 | 11.6 | 59.4 | 29.0 | 26.1 | 73.9 | 0.00828249856101 | 0.02489300000000 |
| MASP1 | MRD_4324/ | 0.0 | 95.9 | 4.1 | 2.0 | 98.0 | 0.0 | 100.0 | 0.0 | 0.0 | 100.0 | 0.02271791227625 | 0.08940100000000 |
| | not known | | | | | | | | | | | | |
| MBLL | rs4238207 | 12.5 | 48.1 | 39.3 | 32.2 | 67.8 | 2.9 | 38.6 | 58.6 | 32.1 | 67.9 | 0.00218537000398 | 0.98900400000000 |
| MBLL | rs9300398 | 46.6 | 12.8 | 40.5 | 66.9 | 33.1 | 38.6 | 2.9 | 58.6 | 67.9 | 32.1 | 0.00303032103263 | 0.82699300000000 |
| NEP | rs9864287 | 36.8 | 18.9 | 44.3 | 59.0 | 41.0 | 29.0 | 7.2 | 63.8 | 60.9 | 39.1 | 0.00479693846587 | 0.67975900000000 |
| PPIC | rs4385206 | 78.7 | 1.7 | 19.6 | 88.5 | 11.5 | 60.0 | 1.4 | 38.6 | 79.3 | 20.7 | 0.00525102317531 | 0.00377900000000 |
| PPID | rs7689418 | 58.1 | 6.4 | 35.5 | 75.8 | 24.2 | 41.4 | 2.9 | 55.7 | 69.3 | 30.7 | 0.00696842005169 | 0.10891500000000 |
| PPID | rs8396 | 58.4 | 6.4 | 35.1 | 76.0 | 24.0 | 42.9 | 2.9 | 54.3 | 70.0 | 30.0 | 0.01099365134327 | 0.14019700000000 |
| RFX3 | rs3012672 | 1.4 | 67.2 | 31.4 | 17.1 | 82.9 | 5.8 | 52.2 | 42.0 | 26.8 | 73.2 | 0.02060709444196 | 0.00842600000000 |
| RFX3 | rs2986678 | 74.0 | 1.7 | 24.3 | 86.1 | 13.9 | 57.1 | 4.3 | 38.6 | 76.4 | 23.6 | 0.02097679403162 | 0.00448000000000 |
| RFX3 | rs2986679 | 74.0 | 1.7 | 24.3 | 86.1 | 13.9 | 57.1 | 4.3 | 38.6 | 76.4 | 23.6 | 0.02097679403162 | 0.00448000000000 |
| SCARB1 | rs10846744 | 1.4 | 74.3 | 24.3 | 13.5 | 86.5 | 2.9 | 52.9 | 44.3 | 25.0 | 75.0 | 0.00276809240197 | 0.00078300000000 |
| SLC22A4 | rs1050152 | 32.8 | 14.9 | 52.4 | 59.0 | 41.0 | 51.4 | 15.7 | 32.9 | 67.9 | 32.1 | 0.00724385258196 | 0.05243500000000 |
| SPOCK | rs11948441 | 55.1 | 6.4 | 38.5 | 74.3 | 25.7 | 48.6 | 0.0 | 51.4 | 74.3 | 25.7 | 0.00476173023570 | 0.99249700000000 |
| SPOCK | rs1528969 | 54.7 | 6.4 | 38.9 | 74.2 | 25.8 | 48.6 | 0.0 | 51.4 | 74.3 | 25.7 | 0.00517591310273 | 0.97472500000000 |
| SPOCK | rs13178069 | 4.8 | 69.0 | 26.2 | 17.9 | 82.1 | 0.0 | 65.7 | 34.3 | 17.1 | 82.9 | 0.02551321470307 | 0.84232400000000 |
| SPOCK | rs3756709 | 0.3 | 73.9 | 25.8 | 13.2 | 86.8 | 2.9 | 81.4 | 15.7 | 10.7 | 89.3 | 0.04324108799332 | 0.42398800000000 |
| SPOCK3 | rs1463611 | 0.7 | 80.7 | 18.6 | 10.0 | 90.0 | 0.0 | 95.7 | 4.3 | 2.1 | 97.9 | 0.00250779939299 | 0.00279200000000 |
| SPOCK3 | rs7658246 | 71.6 | 2.4 | 26.0 | 84.6 | 15.4 | 87.0 | 1.4 | 11.6 | 92.8 | 7.2 | 0.02002807184809 | 0.01279200000000 |
| SPOCK3 | rs1579404 | 3.7 | 58.1 | 38.2 | 22.8 | 77.2 | 12.9 | 54.3 | 32.9 | 29.3 | 70.7 | 0.02340449973216 | 0.10655200000000 |
| SPOCK3 | rs9312522 | 78.7 | 0.7 | 20.6 | 89.0 | 11.0 | 91.4 | 0.0 | 8.6 | 95.7 | 4.3 | 0.02614175695907 | 0.01609400000000 |
| SPOCK3 | rs9996643 | 2.4 | 70.9 | 26.7 | 15.7 | 84.3 | 1.4 | 85.7 | 12.9 | 7.9 | 92.1 | 0.02890729109518 | 0.01670100000000 |
| TLR7 | rs179008 | 68.8 | 12.5 | 18.6 | 78.1 | 21.9 | 68.6 | 24.3 | 7.1 | 72.1 | 27.9 | 0.00626232661600 | 0.12991300000000 |
| TLR7 | rs179011 | 12.8 | 68.2 | 18.9 | 22.3 | 77.7 | 22.9 | 70.0 | 7.1 | 26.4 | 73.6 | 0.01013814946028 | 0.29685600000000 |
| TLR8 | rs1013150 | 12.2 | 68.2 | 19.6 | 22.0 | 78.0 | 17.1 | 78.6 | 4.3 | 19.3 | 80.7 | 0.00197000407670 | 0.48822600000000 |
| TLR8 | rs5744080 | 50.0 | 24.0 | 26.0 | 63.0 | 37.0 | 52.9 | 37.1 | 10.0 | 57.9 | 42.1 | 0.00381856799087 | 0.25888600000000 |
| TLR8 | rs178996 | 8.4 | 74.3 | 17.2 | 17.1 | 82.9 | 14.3 | 81.4 | 4.3 | 16.4 | 83.6 | 0.00501040829282 | 0.85766400000000 |
| TLR8 | rs3827469 | 74.0 | 7.4 | 18.6 | 83.3 | 16.7 | 71.4 | 18.6 | 10.0 | 76.4 | 23.6 | 0.01104596123753 | 0.05803400000000 |
| TLR8 | rs5935445 | 52.4 | 21.6 | 26.0 | 65.4 | 34.6 | 55.7 | 32.9 | 11.4 | 61.4 | 38.6 | 0.01108547003624 | 0.38023100000000 |
| TLR8 | rs5978593 | 36.5 | 31.1 | 32.4 | 52.7 | 47.3 | 48.6 | 35.7 | 15.7 | 56.4 | 43.6 | 0.01322817549187 | 0.42674600000000 |
| TLR8 | rs5741883 | 62.8 | 13.5 | 23.6 | 74.7 | 25.3 | 68.6 | 21.4 | 10.0 | 73.6 | 26.4 | 0.01499932772764 | 0.79014600000000 |
| TLR8 | rs5744088 | 8.8 | 74.7 | 16.6 | 17.1 | 82.9 | 12.9 | 81.4 | 5.7 | 15.7 | 84.3 | 0.03145512538987 | 0.70156600000000 |

TABLE 2B
Raw Data for SNP Genotypes in Control and AAA + AMD Population

| | | | | Number | Number | Number of | | | | | |
|---|---|---|---|---|---|---|---|---|---|---|---|
| | | Number with. | | of Allele1 | of Allele2 | Heterozygotes | Number with. | | | | Number of |
| | | Undetermined- | Size of | Homozygotes | Homozygotes | (Both Alleles) | Undetermined- | | Number of Allele1 | Number of Allele2 | Heterozygotes |
| | | Genotype in | Control | in Control | in Control | in Control | Genotype | Size of AAA | Homozygotes | Homozygotes | (Both Alleles) |
| Gene | SNP | Control Popn. | Population | Population | Population | Population | in AAA Popn. | Population | in AAA Population | in AAA Population | in AAA Population |
| ADAM12 | rs4962543 | 0 | 296 | 141 | 44 | 111 | 0 | 70 | 39 | 1 | 30 |
| ADAM12 | rs1621212 | 0 | 296 | 88 | 51 | 157 | 0 | 70 | 37 | 9 | 24 |
| ADAM12 | rs1676717 | 6 | 290 | 51 | 84 | 155 | 1 | 69 | 9 | 36 | 24 |
| ADAM12 | rs12779767 | 0 | 296 | 124 | 32 | 140 | 0 | 70 | 21 | 18 | 31 |
| ADAM12 | rs11244834 | 1 | 295 | 32 | 122 | 141 | 0 | 70 | 18 | 21 | 31 |
| ADAM12 | rs1674923 | 0 | 296 | 45 | 113 | 138 | 0 | 70 | 19 | 17 | 34 |
| ADAM12 | rs1676736 | 0 | 296 | 113 | 45 | 138 | 0 | 70 | 17 | 19 | 34 |
| ADAM12 | rs1674888 | 0 | 296 | 99 | 53 | 144 | 0 | 70 | 19 | 6 | 45 |
| ADRB2 | rs1800888 | 0 | 296 | 291 | 1 | 4 | 0 | 70 | 63 | 0 | 7 |
| APOD | rs2280520 | 0 | 296 | 7 | 205 | 84 | 0 | 70 | 3 | 60 | 7 |
| APOD | rs4677695 | 0 | 296 | 7 | 207 | 82 | 0 | 70 | 3 | 60 | 7 |
| APOD | rs4677692 | 0 | 296 | 207 | 7 | 82 | 0 | 70 | 60 | 3 | 7 |
| BMP7 | rs6064517 | 0 | 296 | 248 | 3 | 45 | 0 | 70 | 45 | 1 | 24 |
| BMP7 | rs6014959 | 1 | 295 | 246 | 4 | 45 | 0 | 70 | 45 | 1 | 24 |
| BMP7 | rs6064506 | 0 | 296 | 85 | 82 | 129 | 0 | 70 | 14 | 11 | 45 |
| BMP7 | rs6025422 | 0 | 296 | 88 | 78 | 130 | 0 | 70 | 17 | 9 | 44 |
| BMP7 | rs6127984 | 0 | 296 | 42 | 119 | 135 | 0 | 70 | 3 | 33 | 34 |
| BMP7 | rs8116259 | 0 | 296 | 119 | 42 | 135 | 0 | 70 | 33 | 3 | 34 |
| BMP7 | rs162315 | 0 | 296 | 15 | 191 | 90 | 0 | 70 | 4 | 34 | 32 |
| BMP7 | rs162316 | 0 | 296 | 15 | 191 | 90 | 0 | 70 | 4 | 34 | 32 |
| C1NH | MRD_4082/ | 26 | 270 | 0 | 232 | 38 | 5 | 65 | 0 | 63 | 2 |
| | rs28362944 | | | | | | | | | | |
| C1QTNF7 | rs4235376 | 1 | 295 | 2 | 262 | 31 | 0 | 70 | 0 | 52 | 18 |
| C1QTNF7 | rs13116208 | 1 | 295 | 25 | 138 | 132 | 0 | 70 | 15 | 30 | 25 |
| C1QTNF7 | rs16891811 | 6 | 290 | 6 | 219 | 65 | 1 | 69 | 6 | 43 | 20 |
| C1QTNF7 | rs4698382 | 0 | 296 | 236 | 6 | 54 | 0 | 70 | 48 | 0 | 22 |
| C1QTNF7 | rs4505816 | 0 | 296 | 222 | 7 | 67 | 0 | 70 | 43 | 6 | 21 |
| C1QTNF7 | rs2192356 | 5 | 291 | 23 | 138 | 130 | 1 | 69 | 13 | 30 | 26 |
| C1QTNF7 | rs2215809 | 0 | 296 | 10 | 223 | 63 | 0 | 70 | 2 | 43 | 25 |
| C1RL | MRD_4110/ | 0 | 296 | 0 | 281 | 15 | 0 | 70 | 2 | 63 | 5 |
| | rs61917913 | | | | | | | | | | |
| C2-BF | rs4151670 | 0 | 296 | 275 | 0 | 21 | 0 | 70 | 69 | 0 | 1 |
| (factorB) | | | | | | | | | | | |
| C3 | MRD_4273/ | 27 | 269 | 18 | 141 | 110 | 4 | 66 | 1 | 44 | 21 |
| | rs2547438 | | | | | | | | | | |
| C3AR1 | rs10846411 | 1 | 295 | 37 | 131 | 127 | 0 | 70 | 2 | 31 | 37 |
| C5 | rs10116271 | 0 | 296 | 70 | 95 | 131 | 0 | 70 | 16 | 12 | 42 |
| C6 | rs10071904 | 0 | 296 | 7 | 200 | 89 | 0 | 70 | 4 | 55 | 11 |
| C6 | rs6892389 | 0 | 296 | 216 | 6 | 74 | 0 | 70 | 57 | 4 | 9 |
| C6 | rs2910644 | 0 | 296 | 234 | 8 | 54 | 0 | 70 | 45 | 2 | 23 |
| C6 | MRD_4420/ | 0 | 296 | 2 | 280 | 14 | 0 | 70 | 0 | 61 | 9 |
| | rs61733159 | | | | | | | | | | |
| C7 | rs1055021 | 0 | 296 | 0 | 243 | 53 | 0 | 70 | 3 | 55 | 12 |
| C7 | rs2271708 | 0 | 296 | 295 | 0 | 1 | 0 | 70 | 67 | 0 | 3 |
| CLUL1 | rs10502288 | 0 | 296 | 24 | 167 | 105 | 0 | 70 | 4 | 26 | 40 |
| CLUL1 | rs8093432 | 0 | 296 | 193 | 11 | 92 | 0 | 70 | 34 | 4 | 32 |
| COL12A1 | rs4708174 | 0 | 296 | 8 | 211 | 77 | 0 | 70 | 0 | 58 | 12 |
| COL19A1 | rs10485243 | 2 | 294 | 189 | 12 | 93 | 1 | 69 | 34 | 8 | 27 |
| COL19A1 | rs737330 | 0 | 296 | 185 | 12 | 99 | 0 | 70 | 34 | 8 | 28 |
| COL19A1 | rs2145905 | 33 | 263 | 2 | 191 | 70 | 7 | 63 | 4 | 42 | 17 |
| CR3 | rs235328 | 1 | 295 | 168 | 26 | 101 | 0 | 70 | 40 | 1 | 29 |
| ENSG00000000971 | rs10801554 | 0 | 296 | 45 | 117 | 134 | 0 | 70 | 20 | 19 | 31 |
| (CFH) | | | | | | | | | | | |
| ENSG00000000971 | rs1329421 | 0 | 296 | 117 | 45 | 134 | 0 | 70 | 19 | 20 | 31 |
| ENSG00000126759 | rs766775 | 0 | 296 | 39 | 188 | 69 | 0 | 70 | 15 | 49 | 6 |
| (properdin) | | | | | | | | | | | |
| ENSG00000148702 | rs11575688 | 1 | 295 | 0 | 286 | 9 | 1 | 69 | 1 | 61 | 7 |
| (HABP2) | | | | | | | | | | | |
| ENSG00000148702 | rs7080536 | 2 | 294 | 0 | 280 | 14 | 2 | 68 | 0 | 58 | 10 |
| ENSG00000197467 | rs3108966 | 0 | 296 | 134 | 29 | 133 | 0 | 70 | 43 | 3 | 24 |
| ENSG00000197467 | rs3104052 | 1 | 295 | 134 | 29 | 132 | 0 | 70 | 43 | 3 | 24 |
| FBLN2 | rs4684148 | 1 | 295 | 30 | 139 | 126 | 1 | 69 | 3 | 45 | 21 |
| FBN2 | rs331079 | 0 | 296 | 3 | 237 | 56 | 0 | 70 | 0 | 65 | 5 |
| FBN2 | rs10073062 | 0 | 296 | 147 | 17 | 132 | 0 | 70 | 40 | 9 | 21 |
| FBN2 | rs27913 | 0 | 296 | 199 | 9 | 88 | 0 | 70 | 51 | 6 | 13 |
| FBN2 | rs468182 | 0 | 296 | 199 | 9 | 88 | 0 | 70 | 51 | 6 | 13 |
| FCGR2A | rs4657045 | 0 | 296 | 254 | 0 | 42 | 0 | 70 | 50 | 0 | 20 |
| FCGR2A | rs11580574 | 0 | 296 | 255 | 4 | 37 | 0 | 70 | 50 | 2 | 18 |
| FCN1 | rs1071583 | 0 | 296 | 112 | 51 | 133 | 0 | 70 | 38 | 5 | 27 |
| FCN1 | rs2989727 | 0 | 296 | 53 | 106 | 137 | 0 | 70 | 6 | 35 | 29 |
| FCN2 | rs3124953 | 2 | 294 | 16 | 172 | 106 | 1 | 69 | 9 | 47 | 13 |
| FHR5 | MRD_3914/ | 0 | 296 | 296 | 0 | 0 | 3 | 67 | 64 | 0 | 3 |
| | rs41306229 | | | | | | | | | | |
| FHR5 | MRD_3905/ | 0 | 296 | 9 | 171 | 116 | 0 | 70 | 6 | 48 | 16 |
| | not known | | | | | | | | | | |
| FHR5 | rs3748557 | 0 | 296 | 171 | 12 | 113 | 0 | 70 | 47 | 7 | 16 |
| FHR5 | MRD_3906/ | 0 | 296 | 171 | 11 | 114 | 0 | 70 | 48 | 6 | 16 |
| | not known | | | | | | | | | | |
| HS3ST4 | rs9938946 | 0 | 296 | 224 | 9 | 63 | 0 | 70 | 45 | 0 | 25 |
| HS3ST4 | rs4441276 | 0 | 296 | 128 | 21 | 147 | 0 | 70 | 26 | 14 | 30 |
| HS3ST4 | rs7197707 | 0 | 296 | 59 | 93 | 144 | 0 | 70 | 5 | 29 | 36 |
| HS3ST4 | rs3923426 | 0 | 296 | 233 | 8 | 55 | 0 | 70 | 48 | 0 | 22 |
| HS3ST4 | rs9928833 | 0 | 296 | 13 | 204 | 79 | 0 | 70 | 0 | 44 | 26 |
| HS3ST4 | rs4286111 | 0 | 296 | 11 | 190 | 95 | 1 | 69 | 5 | 33 | 31 |
| HS3ST4 | rs11074715 | 0 | 296 | 113 | 37 | 146 | 0 | 70 | 35 | 3 | 32 |
| HS3ST4 | rs4287571 | 0 | 296 | 36 | 116 | 144 | 0 | 70 | 3 | 36 | 31 |
| HS3ST4 | rs8044250 | 7 | 289 | 212 | 11 | 66 | 1 | 69 | 57 | 0 | 12 |
| HS3ST4 | rs4441274 | 0 | 296 | 190 | 11 | 95 | 0 | 70 | 34 | 5 | 31 |
| HS3ST4 | rs4523929 | 0 | 296 | 148 | 38 | 110 | 0 | 70 | 24 | 10 | 36 |
| ITGA6 | rs12471315 | 0 | 296 | 27 | 143 | 126 | 1 | 69 | 0 | 40 | 29 |
| ITGA6 | rs12464480 | 2 | 294 | 22 | 151 | 121 | 0 | 70 | 0 | 43 | 27 |
| ITGA6 | rs10497383 | 0 | 296 | 205 | 14 | 77 | 0 | 70 | 53 | 0 | 17 |
| ITGA6 | rs10497384 | 0 | 296 | 14 | 205 | 77 | 0 | 70 | 0 | 53 | 17 |
| ITGA6 | rs1076594 | 1 | 295 | 126 | 30 | 139 | 0 | 70 | 38 | 2 | 30 |
| MASP1 | rs698086 | 0 | 296 | 36 | 118 | 142 | 1 | 69 | 8 | 41 | 20 |
| MASP1 | MRD_4324/ | 0 | 296 | 0 | 284 | 12 | 0 | 70 | 0 | 70 | 0 |
| | not known | | | | | | | | | | |
| MBLL | rs4238207 | 1 | 295 | 37 | 142 | 116 | 0 | 70 | 2 | 27 | 41 |
| MBLL | rs9300398 | 0 | 296 | 138 | 38 | 120 | 0 | 70 | 27 | 2 | 41 |
| NEP | rs9864287 | 0 | 296 | 109 | 56 | 131 | 1 | 69 | 20 | 5 | 44 |
| PPIC | rs4385206 | 0 | 296 | 233 | 5 | 58 | 0 | 70 | 42 | 1 | 27 |
| PPID | rs7689418 | 0 | 296 | 172 | 19 | 105 | 0 | 70 | 29 | 2 | 39 |
| PPID | rs8396 | 0 | 296 | 173 | 19 | 104 | 0 | 70 | 30 | 2 | 38 |
| RFX3 | rs3012672 | 0 | 296 | 4 | 199 | 93 | 1 | 69 | 4 | 36 | 29 |
| RFX3 | rs2986678 | 0 | 296 | 219 | 5 | 72 | 0 | 70 | 40 | 3 | 27 |
| RFX3 | rs2986679 | 0 | 296 | 219 | 5 | 72 | 0 | 70 | 40 | 3 | 27 |
| SCARB1 | rs10846744 | 0 | 296 | 4 | 220 | 72 | 0 | 70 | 2 | 37 | 31 |
| SLC22A4 | rs1050152 | 0 | 296 | 97 | 44 | 155 | 0 | 70 | 36 | 11 | 23 |
| SPOCK | rs11948441 | 0 | 296 | 163 | 19 | 114 | 0 | 70 | 34 | 0 | 36 |
| SPOCK | rs1528969 | 0 | 296 | 162 | 19 | 115 | 0 | 70 | 34 | 0 | 36 |
| SPOCK | rs13178069 | 2 | 294 | 14 | 203 | 77 | 0 | 70 | 0 | 46 | 24 |
| SPOCK | rs3756709 | 1 | 295 | 1 | 218 | 76 | 0 | 70 | 2 | 57 | 11 |
| SPOCK3 | rs1463611 | 0 | 296 | 2 | 239 | 55 | 0 | 70 | 0 | 67 | 3 |
| SPOCK3 | rs7658246 | 0 | 296 | 212 | 7 | 77 | 1 | 69 | 60 | 1 | 8 |
| SPOCK3 | rs1579404 | 0 | 296 | 11 | 172 | 113 | 0 | 70 | 9 | 38 | 23 |
| SPOCK3 | rs9312522 | 0 | 296 | 233 | 2 | 61 | 0 | 70 | 64 | 0 | 6 |
| SPOCK3 | rs9996643 | 0 | 296 | 7 | 210 | 79 | 0 | 70 | 1 | 60 | 9 |
| TLR7 | rs179008 | 1 | 295 | 203 | 37 | 55 | 0 | 70 | 48 | 17 | 5 |
| TLR7 | rs179011 | 0 | 296 | 38 | 202 | 56 | 0 | 70 | 16 | 49 | 5 |
| TLR8 | rs1013150 | 0 | 296 | 36 | 202 | 58 | 0 | 70 | 12 | 55 | 3 |
| TLR8 | rs5744080 | 0 | 296 | 148 | 71 | 77 | 0 | 70 | 37 | 26 | 7 |
| TLR8 | rs178996 | 0 | 296 | 25 | 220 | 51 | 0 | 70 | 10 | 57 | 3 |
| TLR8 | rs3827469 | 0 | 296 | 219 | 22 | 55 | 0 | 70 | 50 | 13 | 7 |
| TLR8 | rs5935445 | 0 | 296 | 155 | 64 | 77 | 0 | 70 | 39 | 23 | 8 |
| TLR8 | rs5978593 | 0 | 296 | 108 | 92 | 96 | 0 | 70 | 34 | 25 | 11 |
| TLR8 | rs5741883 | 0 | 296 | 186 | 40 | 70 | 0 | 70 | 48 | 15 | 7 |
| TLR8 | rs5744088 | 0 | 296 | 26 | 221 | 49 | 0 | 70 | 9 | 57 | 4 |

TABLE 3A
Exemplary pairwise combinations of informative SNPs for AAA

| | rs3737002 | rs2251252 | rs3742089 | rs3764880 | rs4286111 | rs1126618 | rs9943268 | rs7416639 | rs2227728 |
|---|---|---|---|---|---|---|---|---|---|
| rs3737002 | | X | X | X | X | X | X | X | X |
| rs2251252 | X | | X | X | X | X | X | X | X |
| rs3742089 | X | X | | X | X | X | X | X | X |
| rs3764880 | X | X | X | | X | X | X | X | X |
| rs4286111 | X | X | X | X | | X | X | X | X |
| rs1126618 | X | X | X | X | X | | X | X | X |
| rs9943268 | X | X | X | X | X | X | | X | X |
| rs7416639 | X | X | X | X | X | X | X | | X |
| rs2227728 | X | X | X | X | X | X | X | X | |
| rs2227718 | X | X | X | X | X | X | X | X | X |
| rs17259045 | X | X | X | X | X | X | X | X | X |
| rs4657045 | X | X | X | X | X | X | X | X | X |
| rs6003227 | X | X | X | X | X | X | X | X | X |
| rs1859346 | X | X | X | X | X | X | X | X | X |
| rs3742088 | X | X | X | X | X | X | X | X | X |
| rs3756709 | X | X | X | X | X | X | X | X | X |
| rs3751555 | X | X | X | X | X | X | X | X | X |
| rs11580574 | X | X | X | X | X | X | X | X | X |
| rs6875250 | X | X | X | X | X | X | X | X | X |
| rs629275 | X | X | X | X | X | X | X | X | X |
| rs10755538 | X | X | X | X | X | X | X | X | X |
| rs7757078 | X | X | X | X | X | X | X | X | X |
| rs536485 | X | X | X | X | X | X | X | X | X |
| rs2230205 | X | X | X | X | X | X | X | X | X |
| rs30300 | X | X | X | X | X | X | X | X | X |
| rs4441274 | X | X | X | X | X | X | X | X | X |
| rs12906440 | X | X | X | X | X | X | X | X | X |

| | rs2227718 | rs17259045 | rs4657045 | rs6003227 | rs1859346 | rs3742088 | rs3756709 | rs3751555 | rs11580574 |
|---|---|---|---|---|---|---|---|---|---|
| rs3737002 | X | X | X | X | X | X | X | X | X |
| rs2251252 | X | X | X | X | X | X | X | X | X |
| rs3742089 | X | X | X | X | X | X | X | X | X |
| rs3764880 | X | X | X | X | X | X | X | X | X |
| rs4286111 | X | X | X | X | X | X | X | X | X |
| rs1126618 | X | X | X | X | X | X | X | X | X |
| rs9943268 | X | X | X | X | X | X | X | X | X |
| rs7416639 | X | X | X | X | X | X | X | X | X |
| rs2227728 | X | X | X | X | X | X | X | X | X |
| rs2227718 | | X | X | X | X | X | X | X | X |
| rs17259045 | X | | X | X | X | X | X | X | X |
| rs4657045 | X | X | | X | X | X | X | X | X |
| rs6003227 | X | X | X | | X | X | X | X | X |
| rs1859346 | X | X | X | X | | X | X | X | X |
| rs3742088 | X | X | X | X | X | | X | X | X |
| rs3756709 | X | X | X | X | X | X | | X | X |
| rs3751555 | X | X | X | X | X | X | X | | X |
| rs11580574 | X | X | X | X | X | X | X | X | |
| rs6875250 | X | X | X | X | X | X | X | X | X |
| rs629275 | X | X | X | X | X | X | X | X | X |
| rs10755538 | X | X | X | X | X | X | X | X | X |
| rs7757078 | X | X | X | X | X | X | X | X | X |
| rs536485 | X | X | X | X | X | X | X | X | X |
| rs2230205 | X | X | X | X | X | X | X | X | X |
| rs30300 | X | X | X | X | X | X | X | X | X |
| rs4441274 | X | X | X | X | X | X | X | X | X |
| rs12906440 | X | X | X | X | X | X | X | X | X |

| | rs6875250 | rs629275 | rs10755538 | rs7757078 | rs7757078 | rs7757078 | rs7757078 | rs7757078 | rs7757078 |
|---|---|---|---|---|---|---|---|---|---|
| rs3737002 | X | X | X | X | X | X | X | X | X |
| rs2251252 | X | X | X | X | X | X | X | X | X |
| rs3742089 | X | X | X | X | X | X | X | X | X |
| rs3764880 | X | X | X | X | X | X | X | X | X |
| rs4286111 | X | X | X | X | X | X | X | X | X |
| rs1126618 | X | X | X | X | X | X | X | X | X |
| rs9943268 | X | X | X | X | X | X | X | X | X |
| rs7416639 | X | X | X | X | X | X | X | X | X |
| rs2227728 | X | X | X | X | X | X | X | X | X |
| rs2227718 | X | X | X | X | X | X | X | X | X |
| rs17259045 | X | X | X | X | X | X | X | X | X |
| rs4657045 | X | X | X | X | X | X | X | X | X |
| rs6003227 | X | X | X | X | X | X | X | X | X |
| rs1859346 | X | X | X | X | X | X | X | X | X |
| rs3742088 | X | X | X | X | X | X | X | X | X |
| rs3756709 | X | X | X | X | X | X | X | X | X |
| rs3751555 | X | X | X | X | X | X | X | X | X |
| rs11580574 | X | X | X | X | X | X | X | X | X |
| rs6875250 | | X | X | X | X | X | X | X | X |
| rs629275 | X | | X | X | X | X | X | X | X |
| rs10755538 | X | X | | X | X | X | X | X | X |
| rs7757078 | X | X | X | | X | X | X | X | X |
| rs536485 | X | X | X | X | | X | X | X | X |
| rs2230205 | X | X | X | X | X | | X | X | X |
| rs30300 | X | X | X | X | X | X | | X | X |
| rs4441274 | X | X | X | X | X | X | X | | X |
| rs12906440 | X | X | X | X | X | X | X | X | |

TABLE 3B
Exemplary pairwise combinations of informative SNPs for AAA + AMD

| | rs12779767 | rs11244834 | rs1674923 | rs1676736 | rs10801554 | rs1329421 | rs1071583 | rs1676717 | rs16891811 |
|---|---|---|---|---|---|---|---|---|---|
| rs12779767 | | X | X | X | X | X | X | X | X |
| rs11244834 | X | | X | X | X | X | X | X | X |
| rs1674923 | X | X | | X | X | X | X | X | X |
| rs1676736 | X | X | X | | X | X | X | X | X |
| rs10801554 | X | X | X | X | | X | X | X | X |
| rs1329421 | X | X | X | X | X | | X | X | X |
| rs1071583 | X | X | X | X | X | X | | X | X |
| rs1676717 | X | X | X | X | X | X | X | | X |
| rs16891811 | X | X | X | X | X | X | X | X | |
| rs4505816 | X | X | X | X | X | X | X | X | X |
| rs10485243 | X | X | X | X | X | X | X | X | X |
| rs737330 | X | X | X | X | X | X | X | X | X |
| rs3108966 | X | X | X | X | X | X | X | X | X |
| rs3104052 | X | X | X | X | X | X | X | X | X |
| rs4684148 | X | X | X | X | X | X | X | X | X |
| rs1621212 | X | X | X | X | X | X | X | X | X |
| rs6064517 | X | X | X | X | X | X | X | X | X |
| rs6014959 | X | X | X | X | X | X | X | X | X |
| rs28362944 | X | X | X | X | X | X | X | X | X |
| rs4235376 | X | X | X | X | X | X | X | X | X |
| rs7080536 | X | X | X | X | X | X | X | X | X |
| rs331079 | X | X | X | X | X | X | X | X | X |
| rs4657045 | X | X | X | X | X | X | X | X | X |
| rs11580574 | X | X | X | X | X | X | X | X | X |
| rs4385206 | X | X | X | X | X | X | X | X | X |
| rs3012672 | X | X | X | X | X | X | X | X | X |
| rs2986678 | X | X | X | X | X | X | X | X | X |

| | rs4505816 | rs10485243 | rs737330 | rs3108966 | rs3104052 | rs4684148 | rs1621212 | rs6064517 | rs6014959 |
|---|---|---|---|---|---|---|---|---|---|
| rs12779767 | X | X | X | X | X | X | X | X | X |
| rs11244834 | X | X | X | X | X | X | X | X | X |
| rs1674923 | X | X | X | X | X | X | X | X | X |
| rs1676736 | X | X | X | X | X | X | X | X | X |
| rs10801554 | X | X | X | X | X | X | X | X | X |
| rs1329421 | X | X | X | X | X | X | X | X | X |
| rs1071583 | X | X | X | X | X | X | X | X | X |
| rs1676717 | X | X | X | X | X | X | X | X | X |
| rs16891811 | X | X | X | X | X | X | X | X | X |
| rs4505816 | | X | X | X | X | X | X | X | X |
| rs10485243 | X | | X | X | X | X | X | X | X |
| rs737330 | X | X | | X | X | X | X | X | X |
| rs3108966 | X | X | X | | X | X | X | X | X |
| rs3104052 | X | X | X | X | | X | X | X | X |
| rs4684148 | X | X | X | X | X | | X | X | X |
| rs1621212 | X | X | X | X | X | X | | X | X |
| rs6064517 | X | X | X | X | X | X | X | | X |
| rs6014959 | X | X | X | X | X | X | X | X | |
| rs28362944 | X | X | X | X | X | X | X | X | X |
| rs4235376 | X | X | X | X | X | X | X | X | X |
| rs7080536 | X | X | X | X | X | X | X | X | X |
| rs331079 | X | X | X | X | X | X | X | X | X |
| rs4657045 | X | X | X | X | X | X | X | X | X |
| rs11580574 | X | X | X | X | X | X | X | X | X |
| rs4385206 | X | X | X | X | X | X | X | X | X |
| rs3012672 | X | X | X | X | X | X | X | X | X |
| rs2986678 | X | X | X | X | X | X | X | X | X |

| | rs28362944 | rs4235376 | rs7080536 | rs331079 | rs4657045 | rs11580574 | rs4385206 | rs3012672 | rs2986678 |
|---|---|---|---|---|---|---|---|---|---|
| rs12779767 | X | X | X | X | X | X | X | X | X |
| rs11244834 | X | X | X | X | X | X | X | X | X |
| rs1674923 | X | X | X | X | X | X | X | X | X |
| rs1676736 | X | X | X | X | X | X | X | X | X |
| rs10801554 | X | X | X | X | X | X | X | X | X |
| rs1329421 | X | X | X | X | X | X | X | X | X |
| rs1071583 | X | X | X | X | X | X | X | X | X |
| rs1676717 | X | X | X | X | X | X | X | X | X |
| rs16891811 | X | X | X | X | X | X | X | X | X |
| rs4505816 | X | X | X | X | X | X | X | X | X |
| rs10485243 | X | X | X | X | X | X | X | X | X |
| rs737330 | X | X | X | X | X | X | X | X | X |
| rs3108966 | X | X | X | X | X | X | X | X | X |
| rs3104052 | X | X | X | X | X | X | X | X | X |
| rs4684148 | X | X | X | X | X | X | X | X | X |
| rs1621212 | X | X | X | X | X | X | X | X | X |
| rs6064517 | X | X | X | X | X | X | X | X | X |
| rs6014959 | X | X | X | X | X | X | X | X | X |
| rs28362944 | | X | X | X | X | X | X | X | X |
| rs4235376 | X | | X | X | X | X | X | X | X |
| rs7080536 | X | X | | X | X | X | X | X | X |
| rs331079 | X | X | X | | X | X | X | X | X |
| rs4657045 | X | X | X | X | | X | X | X | X |
| rs11580574 | X | X | X | X | X | | X | X | X |
| rs4385206 | X | X | X | X | X | X | | X | X |
| rs3012672 | X | X | X | X | X | X | X | | X |
| rs2986678 | X | X | X | X | X | X | X | X | |

TABLE 4A
Gene Identifiers Based on the EnsEMBL Database

| Gene Name | Gene ID |
|---|---|
| ADAM12 | ENSG00000148848 |
| ADAMTS19 | ENSG00000145808 |
| ADRB2 | ENSG00000169252 |
| APBA2 | ENSG00000034053 |
| BF (factor B) | ENSG00000166285 |
| BMP7 | ENSG00000101144 |
| C1NH | ENSG00000149131 |
| C1Qa | ENSG00000173372 |
| C1QDC1 | ENSG00000110888 |
| C1QG | ENSG00000159189 |
| C1QR1 | ENSG00000125810 |
| C1QTNF1 | ENSG00000173918 |
| C1QTNF2 | ENSG00000145861 |
| C1QTNF3 | ENS000000113411 |
| C1QTNF5 | ENSG00000184824 |
| C1QTNF6 | ENSG00000133466 |
| C1QTNF7 | ENSG00000163145 |
| C1R | ENSG00000159403 |
| C1RL | ENSG00000139178 |
| C1S | ENSG00000182326 |
| C2 | ENSG00000166278 |
| C2-BF(factor B) | ENSG00000204359 |
| C3 | ENSG00000125730 |
| C3AR1 | ENSG00000171860 |
| C4BPA | ENSG00000123838 |
| C4BPAL2 | ENSG00000123838 |
| C4BPB | ENSG00000123843 |
| C5 | ENSG00000106804 |
| C5R1 | ENSG00000197405 |
| C6 | ENSG00000039537 |
| C7 | ENSG00000112936 |
| C8A | ENSG00000157131 |
| C8B | ENSG00000021852 |
| C8G | ENSG00000176919 |
| C9 | ENSG00000113600 |
| CD97 | ENSG00000123146 |
| CFH | ENSG00000000971 |
| CLU | ENSG00000120885 |
| CLUL1 | ENSG00000079101 |
| COL12A1 | ENSG00000111799 |
| COL13A1 | ENSG00000197467 |
| COL19A1 | ENSG00000082293 |
| CPAMD8 | ENSG00000160111 |
| CPN1 | ENSG00000120054 |
| CPN2 | ENSG00000178772 |
| CR1 | ENSG00000203710 |
| CR1L | ENSG00000197721 |
| CR2 | ENSG00000117322 |
| CR3 | ENSG00000160255 |
| CR3A (ITGAM) | ENSG00000169896 |
| CRP | ENSG00000132693 |
| DAF (CD55) | ENSG00000196352 |
| DF (factor D) | ENSG00000197766 |
| Fl3B | ENSG00000143278 |
| FBLN1 | ENSG00000077942 |
| FBLN2 | ENSG00000163520 |
| FBN2 | ENSG00000138829 |
| FCGR2A | ENSG00000143226 |
| FCN1 | ENSG00000085265 |
| FCN2 | ENSG00000160339 |
| FCN3 | ENSG00000142748 |
| FHR1 (CFHL1/HFL1) | ENSG00000080910 |
| FHR2 (CFHL3/FHL3) | ENSG00000134391 |
| FHR3 | ENSG00000116785 |
| FHR4 | ENSG00000134365 |
| FHR5 | ENSG00000134389 |
| HABP2 | ENSG00000148702 |
| HCK | ENSG00000101336 |
| HP (a & b chains) | ENSG00000197711 |
| HS3ST4 | ENS000000182601 |
| IBSP/integrin-binding sialoprotein | ENSG00000029559 |
| IF | IF |
| IFNAR1 | ENSG00000142166 |
| IFNAR2 | ENSG00000159110 |
| IGLC1 | **ENSG00000211679 |
| ITGA6 | ENSG00000091409 |
| ITGAX | ENSG00000140678 |
| LMAN1 | ENSG00000074695 |
| MASP1 | ENSG00000127241 |
| MASP2 | ENSG00000009724 |
| MBL2 | ENSG00000165471 |
| MBLL | ENSG00000139793 |
| MCP | ENSG00000117335 |
| PPIC | **ENSG00000168938 |
| RFX3 | ENSG00000080298 |
| SCARB1 | ENSG00000073060 |
| SDC4 | ENSG00000124145 |
| SERPINA3 | ENS000000196136 |
| SPOCK | ENSG00000152377 |
| SPOCK3 | ENSG00000196104 |
| TGFBR2 | ENSG00000163513 |
| TLR1 | ENSG00000174125 |
| TLR2 | ENSG00000137462 |
| TLR3 | ENSG00000164342 |
| TLR4 | ENSG00000136869 |
| TLR5 | ENSG00000187554 |
| TLR6 | ENSG00000174130 |
| TLR7 | ENSG00000196664 |
| TLR8 | ENSG00000101916 |
| TLR9 | ENSG00000173366 |
| VTN | ENSG00000109072 |

TABLE 4B
Sequence Information for Allele 1 and
Allele 2 for SNPs in Tables 1A and 2A

| | | Chromo- | |
|---|---|---|---|
| | | some | Allele 1/ |
| Gene | SNP | No. | Allele 2 |
| ADAM12 | rs11244834 | 10 | C/T |
| ADAM12 | rs12779767 | 10 | C/T |
| ADAM12 | rs1621212 | 10 | C/T |
| ADAM12 | rs1674888 | 10 | A/G |
| ADAM12 | rs1674923 | 10 | C/T |
| ADAM12 | rs1676717 | 10 | A/G |
| ADAM12 | rs1676736 | 10 | C/T |
| ADAM12 | rs4962543 | 10 | C/G |
| ADAMTS19 | rs10070537 | 5 | A/G |
| ADAMTS19 | rs10072248 | 5 | C/T |
| ADAMTS19 | rs25816 | 5 | A/G |
| ADAMTS19 | rs25821 | 5 | A/G |
| ADAMTS19 | rs30300 | 5 | C/T |
| ADAMTS19 | rs30693 | 5 | G/T |
| ADAMTS19 | rs6875250 | 5 | A/T |
| ADAMTS2 | rs191415 | 5 | C/T |
| ADAMTS2 | rs459668 | 5 | C/T |
| ADAMTS2 | rs467017 | 5 | A/C |
| ADAMTS2 | rs7704836 | 5 | A/G |
| ADRB2 | rs1800888 | 5 | C/T |
| APBA2 | rs12906440 | 15 | A/C |
| APBA2 | rs3751555 | 15 | C/G |
| APOD | rs2280520 | | A/G |
| APOD | rs4677692 | | A/G |
| APOD | rs4677695 | | A/G |
| BMP7 | rs162315 | 20 | A/G |
| BMP7 | rs162316 | 20 | A/G |
| BMP7 | rs6014959 | 20 | A/G |
| BMP7 | rs6025422 | 20 | A/G |
| BMP7 | rs6064506 | 20 | C/G |
| BMP7 | rs6064517 | 20 | C/T |
| BMP7 | rs6127984 | 20 | A/G |
| BMP7 | rs8116259 | 20 | A/G |
| C1NH | MRD_4082/ | 11 | C/T |
| | rs28362944 | | |
| C1QDC1 | MRD 4087/ | 12 | G/T |
| | rs7299800 | | |
| C1QDC1 | rs10843824 | 12 | C/T |
| C1QDC1 | rs10843831 | 12 | C/T |
| C1QDC1 | rs10843834 | 12 | C/T |
| C1QTNF7 | rs13116208 | 4 | G/T |
| C1QTNF7 | rs16891811 | 4 | A/G |
| C1QTNF7 | rs2192356 | 4 | A/G |
| C1QTNF7 | rs2215809 | 4 | A/C |
| C1QTNF7 | rs4235376 | 4 | C/T |
| C1QTNF7 | rs4505816 | 4 | C/G |
| C1QTNF7 | rs4698382 | 4 | A/G |
| C1RL | MRD_4110/ | 12 | A/G |
| | rs61917913 | | |
| C1RL | rs3742088 | 12 | G/T |
| C1RL | rs3742089 | 12 | A/G |
| C1RL | rs744141 | 12 | C/G |
| C1S | MRD_4094/ | 12 | A/G |
| | not known | | |
| C2-BF(factorB) | rs2072634 | 6 | A/G |
| C2-BF(factorB) | rs4151670 | 6 | C/T |
| C3 | MRD_4273/ | 19 | G/T |
| | rs2547438 | | |
| C3 | rs2230205 | 19 | A/G |
| C3AR1 | rs10846411 | 12 | A/G |
| C4BPA | rs1126618 | 1 | C/T |
| C4BPA | rs7416639 | 1 | A/G |
| C4BPA | rs9943268 | 1 | G/T |
| C5 | rs10116271 | 9 | C/T |
| C6 | MRD_4419/ | 5 | A/T |
| | rs61734263 | | |
| C6 | MRD_4420/ | 5 | G/T |
| | rs61733159 | | |
| C6 | rs10071904 | 5 | A/G |
| C6 | rs2910644 | 5 | C/T |
| C6 | rs6892389 | 5 | A/G |
| C7 | rs1055021 | 5 | A/C |
| C7 | rs2271708 | 5 | A/G |
| C8A | MRD_4044/ | 1 | A/C |
| | not known | | |
| C8A | MRD_4048/ | 1 | C/G |
| | not known | | |
| C9 | MRD_4392/ | 5 | A/G |
| | rs34882957 | | |
| CLUL1 | rs10502288 | 18 | A/G |
| CLUL1 | rs8093432 | 18 | A/G |
| COL12A1 | rs4708174 | 6 | A/C |
| COL19A1 | rs10485243 | 6 | A/T |
| COL19A1 | rs10755538 | 6 | C/T |
| COL19A1 | rs1340975 | 6 | A/T |
| COL19A1 | rs2145905 | 6 | A/G |
| COL19A1 | rs2502560 | 6 | G/T |
| COL19A1 | rs737330 | 6 | A/G |
| COL19A1 | rs7757078 | 6 | C/T |
| CR1 | MRD_3980/ | 1 | A/G |
| | rs17259045 | | |
| CR1 | MRD_3987/ | 1 | A/G |
| | not known | | |
| CR1 | rs11118167 | 1 | C/T |
| CR1 | rs1408078 | 1 | A/G |
| CR1 | rs2274567 | 1 | A/G |
| CR1 | rs3737002 | 1 | C/T |
| CR1 | rs4844599 | 1 | G/T |
| CR1L | MRD_4008/ | 1 | A/G |
| | rs12729569 | | |
| CR3 | rs235328 | 21 | C/G |
| CR3A(ITGAM) | MRD_4127/ | 16 | A/C |
| | rs8051304 | | |
| CR3A(ITGAM) | MRD_4129/ | 16 | C/T |
| | rs3764327 | | |
| CR3A(ITGAM) | rs3925075 | 16 | C/T |
| CR3A(ITGAM) | rs4561481 | 16 | A/G |
| CR3A(ITGAM) | rs7206295 | 16 | C/T |
| CR3A(ITGAM) | rs889551 | 16 | C/T |
| ENSG00000000971 | rs10801554 | 1 | C/T |
| ENSG00000000971 | rs1329421 | 1 | A/T |
| ENSG00000008056- | rs766775 | | A/T |
| ENSG00000126759 | | | |
| ENSG00000029559 | rs17013182 | 4 | A/G |
| ENSG00000148702 | rs11575688 | 10 | C/G |
| ENSG00000148702 | rs2000278 | 10 | G/T |
| ENSG00000148702 | rs2240878 | 10 | G/T |
| ENSG00000148702- | rs7080536 | 10 | A/G |
| ENSG00000197893 | | | |
| ENSG00000197467 | rs3104052 | 10 | C/T |
| ENSG00000197467 | rs3108966 | 10 | G/T |
| FBLN2 | rs4684148 | 3 | A/G |
| FBN2 | rs10073062 | 5 | G/T |
| FBN2 | rs27913 | 5 | C/T |
| FBN2 | rs331079 | 5 | C/G |
| FBN2 | rs468182 | 5 | A/G |
| FCGR2A | rs11580574 | 1 | C/G |
| FCGR2A | rs4657045 | 1 | C/G |
| FCN1 | rs1071583 | 9 | C/T |
| FCN1 | rs2989727 | 9 | C/T |
| FCN2 | rs3124953 | 9 | A/G |
| FHR5 | MRD_3905/ | 1 | A/G |
| | not known | | |
| FHR5 | MRD_3906/ | 1 | C/T |
| | not known | | |
| FHR5 | MRD_3914/ | 1 | C/T |
| | rs41306229 | | |
| FHR5 | rs3748557 | 1 | A/T |
| HS3ST4 | rs11074715 | 16 | A/G |
| HS3ST4 | rs11645232 | 16 | C/G |
| HS3ST4 | rs12103080 | 16 | A/G |
| HS3ST4 | rs3923426 | 16 | C/T |
| HS3ST4 | rs4286111 | 16 | A/G |
| HS3ST4 | rs4287571 | 16 | G/T |
| HS3ST4 | rs4441274 | 16 | A/T |
| HS3ST4 | rs4441276 | 16 | A/G |
| HS3ST4 | rs4523929 | 16 | G/T |
| HS3ST4 | rs6497910 | 16 | C/T |
| HS3ST4 | rs7197707 | 16 | A/G |
| HS3ST4 | rs8044250 | 16 | A/G |
| HS3ST4 | rs9928833 | 16 | C/T |
| HS3ST4 | rs9938946 | 16 | A/G |
| IGLC1 | rs3814997 | 22 | C/T |
| IGLC1 | rs6003227 | 22 | A/G |
| ITGA6 | rs10497383 | 2 | A/C |
| ITGA6 | rs10497384 | 2 | A/G |
| ITGA6 | rs1076594 | 2 | A/G |
| ITGA6 | rs12464480 | 2 | C/T |
| ITGA6 | rs12471315 | 2 | A/T |
| ITGAX | rs11150614 | 16 | A/G |
| MASP1 | MRD_4324/ | 3 | C/T |
| | not known | | |
| MASP1 | rs698086 | 3 | A/G |
| MBLL | rs4238207 | 13 | C/T |
| MBLL | rs9300398 | 13 | C/T |
| NEP | rs9864287 | 3 | A/T |
| PPIC | rs4385206 | 5 | C/T |
| PPID | rs7689418 | 4 | G/T |
| PPID | rs8396 | 4 | A/G |
| RFX3 | rs2986678 | 9 | A/G |
| RFX3 | rs2986679 | 9 | C/T |
| RFX3 | rs3012672 | 9 | C/G |
| RFX3 | rs536485 | 9 | A/C |
| RFX3 | rs559746 | 9 | A/T |
| RFX3 | rs613518 | 9 | C/T |
| RFX3 | rs629275 | 9 | A/G |
| SCARB1 | rs10846744 | 12 | C/G |
| SDC4 | rs2251252 | 20 | A/G |
| SLC22A4 | rs1050152 | 5 | C/T |
| SPOCK | rs10491299 | 5 | C/T |
| SPOCK | rs11948133 | 5 | G/T |
| SPOCK | rs11948441 | 5 | C/T |
| SPOCK | rs12719499 | 5 | C/T |
| SPOCK | rs13178069 | 5 | C/T |
| SPOCK | rs1528969 | 5 | C/T |
| SPOCK | rs1859346 | 5 | A/C |
| SPOCK | rs2905965 | 5 | C/G |
| SPOCK | rs2905972 | 5 | C/T |
| SPOCK | rs3756709 | 5 | C/T |
| SPOCK | rs6873075 | 5 | A/C |
| SPOCK3 | rs10213065 | 4 | A/C |
| SPOCK3 | rs1463611 | 4 | A/G |
| SPOCK3 | rs1579404 | 4 | A/C |
| SPOCK3 | rs7658246 | 4 | A/T |
| SPOCK3 | rs9312522 | 4 | A/G |
| SPOCK3 | rs9996643 | 4 | A/G |
| TLR7 | rs179008 | | A/T |
| TLR7 | rs179011 | | A/C |
| TLR7 | rs5935436 | | C/T |
| TLR7 | rs864058 | | C/T |
| TLR8 | rs1013150 | | A/G |
| TLR8 | rs178996 | | G/T |
| TLR8 | rs3764880 | | A/G |
| TLR8 | rs3827469 | | A/G |
| TLR8 | rs5741883 | | C/T |
| TLR8 | rs5744080 | | C/T |
| TLR8 | rs5744088 | | C/G |
| TLR8 | rs5935445 | | G/T |
| TLR8 | rs5978593 | | A/G |
| VTN | MRD_4187/ | 17 | A/C |
| | rs2227718 | | |
| VTN | rs2227728 | 17 | C/T |

TABLE 4C
Sequence Information for Some Predictive SNPs

| | | Chromo- | | |
|---|---|---|---|---|
| | | some | Allele 1/ | |
| Gene | SNP | No. | Allele 2 | SNP Flanking Sequence |
| ADAM12 | rs11244834 | 10 | C/T | actctgctgtaagctctattttccac[c/t]tgctattttcttccacactgaccca |
| ADAM12 | rs12779767 | 10 | C/T | tgtatgtgtgtgtatgtgggcacgtg[c/t]gtatatttgtgtgtgtgcatgtgca |
| ADAM12 | rs1621212 | 10 | C/T | gattttattttaaattctaagcagat[c/t]atgttttcatttttacaaagagatt |
| ADAM12 | rs1674923 | 10 | C/T | cctgccaccacactgtgctcactttt[c/t]cctctagctcatgctactctagcca |
| ADAM12 | rs1676717 | 10 | A/G | taaaatgctctgtgcctcttaagcag[a/g]atttatatgctgaggaatatatttt |
| ADAM12 | rs1676736 | 10 | C/T | tttagaatttgtgctcttaaccactg[c/t]gtggcgctaccagaccttacaggat |
| ADAM12 | rs4962543 | 10 | C/G | atggaaacagtcctccaagggacagg[c/g]tatgtctagacgcaatccagacccc |
| ADAMTS19 | rs30300 | 5 | C/T | attgttggtgccacaattttgtgacc[c/t]ttggtaaggtattaagcctctgtgt |
| ADAMTS19 | rs6875250 | 5 | A/T | atccttatgtgccactgattctttaa[a/t]taccatcatatctgctgtgccatat |
| ADRB2 | rs1800888 | 5 | C/T | gatggtgtggattgtgtcaggcctta[c/t]ctccttcttgcccattcagatgcac |
| APBA2 | rs12906440 | 15 | A/C | gcaccaggtgtcagctgggcaacgcc[a/c]cgctgcaactggaggtgccagcaat |
| APBA2 | rs3751555 | 15 | C/G | accctcccacccggctgcatacccgg[c/g]cagggctcccacagagacaaggagg |
| APBA2 | rs3829467 | 15 | C/T | gtggaagacaccctctggtccccctg[c/t]gcccccatgccaggctcatgggctc |
| BMP7 | rs6014959 | 20 | A/G | ggctcagggaggccgggtaactttca[a/g]aggtcacaaatcaggtgagcggctg |
| BMP7 | rs6064517 | 20 | C/T | gcatggttgtcctttaaacctctttc[c/t]ggtgtgggaagcaggagaatatgag |
| C1NH | MRD_4082/ | 11 | C/T | gctccgaggctggctggctccgcagg[c/t]ccgctgacgtcgccgcccagatggc |
| | rs28362944 | | | |
| C1QTNF7 | rs16891811 | 4 | A/G | cttttataagtatttcaaatcaaatt[a/g]tgggtaatgactgggaagtagttaa |
| C1QTNF7 | rs4235376 | 4 | C/T | tcccatgcccattatcagttttgaaa[c/t]gggtcaggaaaagctaagctagctg |
| C1QTNF7 | rs4505816 | 4 | C/G | gaaactgaagcccaacaattgggatt[c/g]tcatctctaagagaattgacttttt |
| C1RL | MRD_4110/ | 12 | A/G | atggcctcagagcccctgctggcctc[a/g]ctgatgggctgactatagttcacag |
| | rs61917913 | | | |
| C1RL | rs3742088 | 12 | G/T | tcccgctttcagatctcattcgtcgg[g/t]tcggatccaagccagttctgtggtc |
| C1RL | rs3742089 | 12 | A/G | gatggatcctcactgctgcccacacc[a/g]tctaccccaaggacagtgtttctct |
| C3 | rs2230205 | 19 | A/G | gtgctgaataagaagaacaaactgac[a/g]cagagtaaggtaagggccagtgacc |
| C4BPA | rs1126618 | 1 | C/T | tgcactgtggagaatgaaacaatagg[c/t]gtttggagaccaagccctcctacct |
| C4BPA | rs7416639 | 1 | A/G | atcaggattagtcacaccaaccatca[a/g]agtggactccttctttgccttacct |
| C4BPA | rs9943268 | 1 | G/T | ttaggattatctggtttgtaatcaca[g/t]catttcaatgattcttttacctcct |
| COL12A1 | rs554152 | 6 | A/G | cacagcagactaaagcatccttgtta[a/g]gccaaataaaaggagtcttccacca |
| COL19A1 | rs10485243 | 6 | A/T | cattcaaggtattctgagggtatttt[a/t]acaagtgatcaaatgtttcactgag |
| COL19A1 | rs10755538 | 6 | C/T | cagagtccctgctagagcacttccca[c/t]caggctgattgaatcccaggttcca |
| COL19A1 | rs737330 | 6 | A/G | caccagaggctaggagagagacctgg[a/g]agatattcctccctagtgcctttgg |
| COL19A1 | rs7757078 | 6 | C/T | cactgacacagattagttgtataatc[c/t]ctagaatttcgtataaatggaatta |
| CR1 | MRD_3980/ | 1 | A/G | cagcaacaatagaacatcttttcaca[a/g]tggaacggtggtaacttaccagtgc |
| | rs17259045 | | | |
| CR1 | rs3737002 | 1 | C/T | agagcagtttccatttgccagtccta[c/t]gatcccaattaatgactttgagttt |
| CR1L | MRD_3991/ | 1 | A/G | agtctactatataatatgaaatatct[a/g]tgagaaaatacgtcttctttatggt |
| | rs2147021 | | | |
| CR1L | MRD_3996/ | 1 | C/T | ccgttttctatcatctgcctaaaaaa[c/t]tcagtctggacaagtgctaaggaca |
| | rs34509370 | | | |
| CR1L | MRD_4008/ | 1 | A/G | ttttggctggaatggaaagcctttgg[a/g]atagcagtgttccagtgtgtgaacg |
| | rs12729569 | | | |
| ENSG00000000971 | rs1329421 | 1 | A/T | cattgttaaatttcatcttattagat[a/t]cagcttagcacataagagtctcttt |
| ENSG00000000971 | rs10801554 | 1 | C/T | catgaattaactatgttatttttctg[c/t]gcggtatcatcaaagaaaaattttt |
| (CFH) | | | | |
| ENSG00000029559 | rs17013182 | 4 | A/G | cttccccaccttttgggaaaaccacc[a/g]ccgttgaatacgagggggagtacga |
| (TBSP/ | | | | |
| integrin- | | | | |
| binding | | | | |
| sialoprotein) | | | | |
| ENSG00000148702 | rs7080536 | 10 | A/G | gatagtgagctggggcctggagtgtg[a/g]gaagaggccaggggtctacacccaa |
| ENSG00000148702 | rs11575688 | 10 | C/G | aatttcatgagcagagctttagggtg[c/g]agaagatattcaagtacagccacta |
| (HABP2) | | | | |
| ENSG00000197467 | rs3104052 | 10 | C/T | cgaaggtcagccctcctccagaaggc[c/t]gcaggtcctctgtcctctacttggc |
| ENSG00000197467 | rs3108966 | 10 | G/T | aaccccacttctcttcctccactgtg[g/t]ctttgacagcatcaaaatccttcct |
| FBLN1 | rs1985671 | 22 | A/C | cactcttcttgcccaggctggtgtgc[a/c]atactccgatctcagctcactgcaa |
| FBLN2 | rs4684148 | 3 | A/G | gggggtgggcgagctgtgggtgaccc[a/g]gcctatcctccctgcaggaagtgcg |
| FBN2 | rs331079 | 5 | C/G | gggaaatttttcctgaatccatcaaa[c/g]tgcaatttcctgaccactgtcttat |
| FCGR2A | rs11580574 | 1 | C/G | tcctcctttcccaggtgttgcgttct[c/g]tcttgggctgagtggcgaggtotct |
| FCGR2A | rs4657045 | 1 | C/G | aaatgagatcccaaatgtctcagaaa[c/g]aatgataaataattttgattggtat |
| FCN1 | rs1071583 | 9 | C/T | tgacagtcggcgtaccaccaggctcc[c/t]tggaacttctcagcacaattcgaag |
| HS3ST4 | rs11074715 | 16 | A/G | aaatgcattgattttaccagatacac[a/g]cacctagtctgggcaagagcctccc |
| HS3ST4 | rs4286111 | 16 | A/G | attgattgattgatttgttttttcca[a/g]taagtcaatatttactgagctgggc |
| HS3ST4 | rs4287571 | 16 | G/T | tggtattatttgcagtaagagtaacc[g/t]gcaagaggctaccttctgatcctgc |
| HS3ST4 | rs4441274 | 16 | A/T | cactgcctgcacagaaagatctgatg[a/t]gcagctctagctttcaatcctgttc |
| IGLC1 | rs3814997 | 22 | C/T | tgatgcgggtttgatttcagtgtttc[c/t]acatatatacttttgtattttattg |
| IGLC1 | rs6003227 | 22 | A/G | ttattcctggggctcactccagccct[a/g]gcaagtagcaagatatcctggggtt |
| ITGA6 | rs12464480 | 2 | C/T | atacaaatgaaacgatccacacacaa[c/t]aaaaagagtttccaggaaattcatg |
| MASP1 | rs698086 | 3 | A/G | tctcttgataagttcaagcatgagtg[a/g]cacgtgatagtgaagtctcaccatg |
| PPIC | rs4385206 | 5 | C/T | gaattgtttaggattttctccataga[c/t]aattatgtcatccatgaataatgac |
| RFX3 | rs2986678 | 9 | A/G | tgcttttaataaggtcacttgtgacc[a/g]cagctaaataccaagctaaaagact |
| RFX3 | rs2986679 | 9 | C/T | gtctgaatttggctttgacaaaaata[c/t]acttgcagttggaaatgggggagac |
| RFX3 | rs3012672 | 9 | C/G | tttataacaacattctgcatcttatt[c/g]caaagtagccaagaagcccaaataa |
| RFX3 | rs536485 | 9 | A/C | aaggtagacaaaacttgaagactggg[a/c]tgaggtgtccacaaatctgagcaca |
| RFX3 | rs629275 | 9 | A/G | tagttcaatagtttgataattttcca[a/g]tgagaaaacacattttgagaatctc |
| SCARB1 | rs10846744 | 12 | C/G | ataattagcttatcaggtttattgct[c/g]tccatctgtatcacctgcctggcca |
| SDC4 | rs2251252 | 20 | A/G | tgggaagtgggggagggaggaaggat[a/g]gctgtagaaaggtcaaagccagaaa |
| SPOCK | rs11948133 | 5 | G/T | ggcctatctcatctttcaatatgcct[g/t]tgtcgctaagcacgatcatttctaa |
| SPOCK | rs1859346 | 5 | A/C | caattttcttgttgttggtttgagtt[a/c]aaatgacctgtcacacacttgtccc |
| SPOCK | rs3756709 | 5 | C/T | ttgaatgcagtaggtaagaagttagt[c/t]agagtagcaactgttgagatcccga |
| SPOCK3 | rs1463611 | 4 | A/G | gtgactttggcaggatattatgactc[a/g]tgtgtgctttggtcttctcgttagt |
| SPOCK3 | rs7658246 | 4 | A/T | ataggtagcagttgtaatggtttcag[a/t]attgtatttgttgttactactgttt |
| SPOCK3 | rs9312522 | 4 | A/G | tgtgtgatgactttcaggtgaattct[a/g]ggacaaggtgattgtcctagatttt |
| TGFBR2 | rs2116142 | 3 | C/T | taataaccagacacatggacatctta[c/t]tccccctgatatgacgcactgaaga |
| TLR8 | rs3764880 | | A/G | gaatgaaaaattagaacaacagaaac[a/g]tggtaagccacttctatttctttag |
| VTN | MRD_4187/ | 17 | A/C | ggtgatgggaggatttcagaagttct[g/t]tggacacctgaaattgggcacaaaa |
| | rs2227718 | | | |

TABLE 4D
Identifying Information SNPs With MRD Designations

MRD_4094, A/G, Human NCBI Build 35, 12, 7044154, C1S, ENSG00000182326,
12: 7044154, ggagcctgcgaaggcaaaatatgtctttagaNatgtggtgcagataacctgtctggatgggtt, 7044038,
7044225,
ctatttagtaattttttcctcctgtcccaacttctgttctttcaagcaatgccctgccctaaggaagacactcccaattctgtttgggagcctgc
gaaggcaaaatatgtctttagagatgtggtgcagataacctgtctggatgggtttgaagttgtggaggtaaagtaccaccttggcttctcc
cca
MRD_4048, C/G, Human NCBI Build 35, 1, 57059265, C8A, ENSG00000157131,
1: 57059265, agcttcgatatgactccacctgtgaacgtctNtactatggagatgatgagaaatactttcgga, 57059112,
57059349,
tatttagaagctgctttgaccatgtaggtacaatatcctgacccaggaagatgctcagagtgtgtacgatgccagttattatgggggcca
gtgtgagacggtatacaatggggaatggagggagcttcgatatgactccacctgtgaacgtetctactatggagatgatgagaaatact
ttcggaaaccctacaactttctgaagtaccactttgaagtaagtctgaacagaggggct
MRD_4044, A/C, Human NCBI Build 35, 1, 57045332, C8A, ENSG00000157131,
1: 57045332, aggagagtaagacgggcagctacacccgcagNagttacctgccagctgagcaactggtcagag,
57045257, 57045416,
taaattttgcatctcaaaattgatgcatggatcttccctttctttaggagagtaagacgggcagctacacccgcagcagttacctgccagct
gagcaactggtcagagtggacagattgctttccgtgccaggacaaaaaggtgagacacttacaaccggt
MRD_3987, A/G, Human NCBI Build 35, 1, 204179829, CR1, CR1exon34, 1: 204179829,
aacttgttcttagcctgcccacatccacccaNgatccaaaacgggcattacattggaggacac, 204179791, 204180012,
tgtgtgggaacttgttcttagcctgcccacatccacccaagatccaaaacgggcattacattggaggacacgtatctctatatcttcctgg
gatgacaatcagctacatttgtgaccccggctacctgttagtgggaaagggcttcattttctgtacagaccagggaatctggagccaatt
ggatcattattgcaaaggtgacttatttcttggtattcctta
MRD_4324, C/T, Human NCBI Build 35, 3, 188461217, MASP1, ENSG00000127241,
3: 188461217, atgtggcctataagggcaatgcatacaatcaNtggtaggctctacctcggcaggtcctgttgt, 188461208,
188461403,
catacaatcattggtaggctctacctcggcaggtcctgttgtctgtgtggaggatgtagccgaagcggcaggagcagtagtagccgcc
aatgtagttgtggcagtagtggtcacaggacagctcctcgtcctccctctccttgcactcgtccacatctgtagggcaggtaaagcctct
ccatcaatacatgcatgat
Note: Each entry sets forth the following data in order, separated by commas:
External ID, Target Allele, Genome Map, Chrom Name, Chrom Position, Gene, Gene Name, Chrom Pos, SNP Flanking Sequence, Amp Min, Amp Max, Amplicon

1. A method of determining an individual's risk of abdominal aortic aneurysm(AAA) or a AAA-associated vascular disorder comprising screening the genome of the individual for the presence or absence of a genetic profile characterized by at least one polymorphism selected from Table 1A and/or Table 2A associated with increased risk for or protection against AAA, wherein the presence of a said genetic profile is indicative of the individual's relative risk of AAA.

2. The method of claim 1, wherein the risk of AAA is determined.

3. The method of claim 1, wherein the genetic profile comprises at least one polymorphism selected from Table 1A.

4-21. (canceled)

22. A method for treating AAA, the method comprising (i) identifying an individual as having a genetic profile characterized by polymorphisms indicative of risk for developing AAA, wherein the genetic profile comprises at least one polymorphism selected from Table 1A or Table 2A, and (ii) treating the individual.

23. The method of claim 22, wherein the genetic profile comprises at least one polymorphism selected from Table 1A.

24. A kit for determining an individual's risk of abdominal aortic aneurysm (AAA) or an AAA-associated vascular disorder in a human subject, the kit comprising reagents for screening, in a sample from the subject, for the presence or absence of a genetic profile in the genome of the subject characterized by at least one polymorphism selected from Table 1A and/or Table 2A associated with increased risk for or protection against AAA, wherein the presence of a said genetic profile is indicative of the individual's relative risk of AAA.


SEQUENCE LISTING (84 sequences)

SEQ ID NO: 1 - DNA, 52 bp, Artificial Sequence
  Synthetic polynucleotide
actctgctgtaagctctattttccacytgctattttcttccacactgaccca

SEQ ID NO: 2 - DNA, 52 bp, Artificial Sequence
  Synthetic polynucleotide
tgtatgtgtgtgtatgtgggcacgtgygtatatttgtgtgtgtgcatgtgca

SEQ ID NO: 3 - DNA, 52 bp, Artificial Sequence
  Synthetic polynucleotide
gattttattttaaattctaagcagatyatgttttcatttttacaaagagatt

SEQ ID NO: 4 - DNA, 52 bp, Artificial Sequence
  Synthetic polynucleotide
cctgccaccacactgtgctcacttttycctctagctcatgctactctagcca

SEQ ID NO: 5 - DNA, 52 bp, Artificial Sequence
  Synthetic polynucleotide
taaaatgctctgtgcctcttaagcagratttatatgctgaggaatatatttt

SEQ ID NO: 6 - DNA, 52 bp, Artificial Sequence
  Synthetic polynucleotide
tttagaatttgtgctcttaaccactgygtggcgctaccagaccttacaggat

SEQ ID NO: 7 - DNA, 52 bp, Artificial Sequence
  Synthetic polynucleotide
atggaaacagtcctccaagggacaggstatgtctagacgcaatccagacccc

SEQ ID NO: 8 - DNA, 52 bp, Artificial Sequence
  Synthetic polynucleotide
attgttggtgccacaattttgtgaccyttggtaaggtattaagcctctgtgt

SEQ ID NO: 9 - DNA, 52 bp, Artificial Sequence
  Synthetic polynucleotide
atccttatgtgccactgattctttaawtaccatcatatctgctgtgccatat

SEQ ID NO: 10 - DNA, 52 bp, Artificial Sequence
  Synthetic polynucleotide
gatggtgtggattgtgtcaggccttayctccttcttgcccattcagatgcac

SEQ ID NO: 11 - DNA, 52 bp, Artificial Sequence
  Synthetic polynucleotide
gcaccaggtgtcagctgggcaacgccmcgctgcaactggaggtgccagcaat

SEQ ID NO: 12 - DNA, 52 bp, Artificial Sequence
  Synthetic polynucleotide
accctcccacccggctgcatacccggscagggctcccacagagacaaggagg

SEQ ID NO: 13 - DNA, 52 bp, Artificial Sequence
  Synthetic polynucleotide
gtggaagacaccctctggtccccctgygcccccatgccaggctcatgggctc

SEQ ID NO: 14 - DNA, 52 bp, Artificial Sequence
  Synthetic polynucleotide
ggctcagggaggccgggtaactttcaraggtcacaaatcaggtgagcggctg

SEQ ID NO: 15 - DNA, 52 bp, Artificial Sequence
  Synthetic polynucleotide
gcatggttgtcctttaaacctctttcyggtgtgggaagcaggagaatatgag

SEQ ID NO: 16 - DNA, 52 bp, Artificial Sequence
  Synthetic polynucleotide
gctccgaggctggctggctccgcaggyccgctgacgtcgccgcccagatggc

SEQ ID NO: 17 - DNA, 52 bp, Artificial Sequence
  Synthetic polynucleotide
cttttataagtatttcaaatcaaattrtgggtaatgactgggaagtagttaa

SEQ ID NO: 18 - DNA, 52 bp, Artificial Sequence
  Synthetic polynucleotide
tcccatgcccattatcagttttgaaaygggtcaggaaaagctaagctagctg

SEQ ID NO: 19 - DNA, 52 bp, Artificial Sequence
  Synthetic polynucleotide
gaaactgaagcccaacaattgggattstcatctctaagagaattgacttttt

SEQ ID NO: 20 - DNA, 52 bp, Artificial Sequence
  Synthetic polynucleotide
atggcctcagagcccctgctggcctcrctgatgggctgactatagttcacag

SEQ ID NO: 21 - DNA, 52 bp, Artificial Sequence
  Synthetic polynucleotide
tcccgctttcagatctcattcgtcggktcggatccaagccagttctgtggtc

SEQ ID NO: 22 - DNA, 52 bp, Artificial Sequence
  Synthetic polynucleotide
gatggatcctcactgctgcccacaccrtctaccccaaggacagtgtttctct

SEQ ID NO: 23 - DNA, 52 bp, Artificial Sequence
  Synthetic polynucleotide
gtgctgaataagaagaacaaactgacrcagagtaaggtaagggccagtgacc

SEQ ID NO: 24 - DNA, 52 bp, Artificial Sequence
  Synthetic polynucleotide
tgcactgtggagaatgaaacaataggygtttggagaccaagccctcctacct

SEQ ID NO: 25 - DNA, 52 bp, Artificial Sequence
  Synthetic polynucleotide
atcaggattagtcacaccaaccatcaragtggactccttctttgccttacct

SEQ ID NO: 26 - DNA, 52 bp, Artificial Sequence
  Synthetic polynucleotide
ttaggattatctggtttgtaatcacakcatttcaatgattcttttacctcct

SEQ ID NO: 27 - DNA, 52 bp, Artificial Sequence
  Synthetic polynucleotide
cacagcagactaaagcatccttgttargccaaataaaaggagtcttccacca

SEQ ID NO: 28 - DNA, 52 bp, Artificial Sequence
  Synthetic polynucleotide
cattcaaggtattctgagggtattttwacaagtgatcaaatgtttcactgag

SEQ ID NO: 29 - DNA, 52 bp, Artificial Sequence
  Synthetic polynucleotide
cagagtccctgctagagcacttcccaycaggctgattgaatcccaggttcca

SEQ ID NO: 30 - DNA, 52 bp, Artificial Sequence
  Synthetic polynucleotide
caccagaggctaggagagagacctggragatattcctccctagtgcctttgg

SEQ ID NO: 31 - DNA, 52 bp, Artificial Sequence
  Synthetic polynucleotide
cactgacacagattagttgtataatcyctagaatttcgtataaatggaatta

SEQ ID NO: 32 - DNA, 52 bp, Artificial Sequence
  Synthetic polynucleotide
cagcaacaatagaacatcttttcacartggaacggtggtaacttaccagtgc

SEQ ID NO: 33 - DNA, 52 bp, Artificial Sequence
  Synthetic polynucleotide
agagcagtttccatttgccagtcctaygatcccaattaatgactttgagttt

SEQ ID NO: 34 - DNA, 52 bp, Artificial Sequence
  Synthetic polynucleotide
agtctactatataatatgaaatatctrtgagaaaatacgtcttctttatggt

SEQ ID NO: 35 - DNA, 52 bp, Artificial Sequence
  Synthetic polynucleotide
ccgttttctatcatctgcctaaaaaaytcagtctggacaagtgctaaggaca

SEQ ID NO: 36 - DNA, 52 bp, Artificial Sequence
  Synthetic polynucleotide
ttttggctggaatggaaagcctttggratagcagtgttccagtgtgtgaacg

SEQ ID NO: 37 - DNA, 52 bp, Artificial Sequence
  Synthetic polynucleotide
cattgttaaatttcatcttattagatwcagcttagcacataagagtctcttt

SEQ ID NO: 38 - DNA, 52 bp, Artificial Sequence
  Synthetic polynucleotide
catgaattaactatgttatttttctgygcggtatcatcaaagaaaaattttt

SEQ ID NO: 39 - DNA, 52 bp, Artificial Sequence
  Synthetic polynucleotide
cttccccaccttttgggaaaaccaccrccgttgaatacgagggggagtacga

SEQ ID NO: 40 - DNA, 52 bp, Artificial Sequence
  Synthetic polynucleotide
gatagtgagctggggcctggagtgtgrgaagaggccaggggtctacacccaa

SEQ ID NO: 41 - DNA, 52 bp, Artificial Sequence
  Synthetic polynucleotide
aatttcatgagcagagctttagggtgsagaagatattcaagtacagccacta

SEQ ID NO: 42 - DNA, 52 bp, Artificial Sequence
  Synthetic polynucleotide
cgaaggtcagccctcctccagaaggcygcaggtcctctgtcctctacttggc

SEQ ID NO: 43 - DNA, 52 bp, Artificial Sequence
  Synthetic polynucleotide
aaccccacttctcttcctccactgtgkctttgacagcatcaaaatccttcct

SEQ ID NO: 44 - DNA, 52 bp, Artificial Sequence
  Synthetic polynucleotide
cactcttcttgcccaggctggtgtgcmatactccgatctcagctcactgcaa

SEQ ID NO: 45 - DNA, 52 bp, Artificial Sequence
  Synthetic polynucleotide
gggggtgggcgagctgtgggtgacccrgcctatcctccctgcaggaagtgcg

SEQ ID NO: 46 - DNA, 52 bp, Artificial Sequence
  Synthetic polynucleotide
gggaaatttttcctgaatccatcaaastgcaatttcctgaccactgtcttat

SEQ ID NO: 47 - DNA, 52 bp, Artificial Sequence
  Synthetic polynucleotide
tcctcctttcccaggtgttgcgttctstcttgggctgagtggcgaggtctct

SEQ ID NO: 48 - DNA, 52 bp, Artificial Sequence
  Synthetic polynucleotide
aaatgagatcccaaatgtctcagaaasaatgataaataattttgattggtat

SEQ ID NO: 49 - DNA, 52 bp, Artificial Sequence
  Synthetic polynucleotide
tgacagtcggcgtaccaccaggctccytggaacttctcagcacaattcgaag

SEQ ID NO: 50 - DNA, 52 bp, Artificial Sequence
  Synthetic polynucleotide
aaatgcattgattttaccagatacacrcacctagtctgggcaagagcctccc

SEQ ID NO: 51 - DNA, 52 bp, Artificial Sequence
  Synthetic polynucleotide
attgattgattgatttgttttttccartaagtcaatatttactgagctgggc

SEQ ID NO: 52 - DNA, 52 bp, Artificial Sequence
  Synthetic polynucleotide
tggtattatttgcagtaagagtaacckgcaagaggctaccttctgatcctgc

SEQ ID NO: 53 - DNA, 52 bp, Artificial Sequence
  Synthetic polynucleotide
cactgcctgcacagaaagatctgatgwgcagctctagctttcaatcctgttc

SEQ ID NO: 54 - DNA, 52 bp, Artificial Sequence
  Synthetic polynucleotide
tgatgcgggtttgatttcagtgtttcyacatatatacttttgtattttattg

SEQ ID NO: 55 - DNA, 52 bp, Artificial Sequence
  Synthetic polynucleotide
ttattcctggggctcactccagccctrgcaagtagcaagatatcctggggtt

SEQ ID NO: 56 - DNA, 52 bp, Artificial Sequence
  Synthetic polynucleotide
atacaaatgaaacgatccacacacaayaaaaagagtttccaggaaattcatg

SEQ ID NO: 57 - DNA, 52 bp, Artificial Sequence
  Synthetic polynucleotide
tctcttgataagttcaagcatgagtgrcacgtgatagtgaagtctcaccatg

SEQ ID NO: 58 - DNA, 52 bp, Artificial Sequence
  Synthetic polynucleotide
gaattgtttaggattttctccatagayaattatgtcatccatgaataatgac

SEQ ID NO: 59 - DNA, 52 bp, Artificial Sequence
  Synthetic polynucleotide
tgcttttaataaggtcacttgtgaccrcagctaaataccaagctaaaagact

SEQ ID NO: 60 - DNA, 52 bp, Artificial Sequence
  Synthetic polynucleotide
gtctgaatttggctttgacaaaaatayacttgcagttggaaatgggggagac

SEQ ID NO: 61 - DNA, 52 bp, Artificial Sequence
  Synthetic polynucleotide
tttataacaacattctgcatcttattscaaagtagccaagaagcccaaataa

SEQ ID NO: 62 - DNA, 52 bp, Artificial Sequence
  Synthetic polynucleotide
aaggtagacaaaacttgaagactgggmtgaggtgtccacaaatctgagcaca

SEQ ID NO: 63 - DNA, 52 bp, Artificial Sequence
  Synthetic polynucleotide
tagttcaatagtttgataattttccartgagaaaacacattttgagaatctc

SEQ ID NO: 64 - DNA, 52 bp, Artificial Sequence
  Synthetic polynucleotide
ataattagcttatcaggtttattgctstccatctgtatcacctgcctggcca

SEQ ID NO: 65 - DNA, 52 bp, Artificial Sequence
  Synthetic polynucleotide
tgggaagtgggggagggaggaaggatrgctgtagaaaggtcaaagccagaaa

SEQ ID NO: 66 - DNA, 52 bp, Artificial Sequence
  Synthetic polynucleotide
ggcctatctcatctttcaatatgcctktgtcgctaagcacgatcatttctaa

SEQ ID NO: 67 - DNA, 52 bp, Artificial Sequence
  Synthetic polynucleotide
caattttcttgttgttggtttgagttmaaatgacctgtcacacacttgtccc

SEQ ID NO: 68 - DNA, 52 bp, Artificial Sequence
  Synthetic polynucleotide
ttgaatgcagtaggtaagaagttagtyagagtagcaactgttgagatcccga

SEQ ID NO: 69 - DNA, 52 bp, Artificial Sequence
  Synthetic polynucleotide
gtgactttggcaggatattatgactcrtgtgtgctttggtcttctcgttagt

SEQ ID NO: 70 - DNA, 52 bp, Artificial Sequence
  Synthetic polynucleotide
ataggtagcagttgtaatggtttcagwattgtatttgttgttactactgttt

SEQ ID NO: 71 - DNA, 52 bp, Artificial Sequence
  Synthetic polynucleotide
tgtgtgatgactttcaggtgaattctrggacaaggtgattgtcctagatttt

SEQ ID NO: 72 - DNA, 52 bp, Artificial Sequence
  Synthetic polynucleotide
taataaccagacacatggacatcttaytccccctgatatgacgcactgaaga

SEQ ID NO: 73 - DNA, 52 bp, Artificial Sequence
  Synthetic polynucleotide
gaatgaaaaattagaacaacagaaacrtggtaagccacttctatttctttag

SEQ ID NO: 74 - DNA, 52 bp, Artificial Sequence
  Synthetic polynucleotide
ggtgatgggaggatttcagaagttctktggacacctgaaattgggcacaaaa

SEQ ID NO: 75 - DNA, 63 bp, Artificial Sequence
  Synthetic polynucleotide
ggagcctgcgaaggcaaaatatgtctttaganatgtggtgcagataacctgtctggatgggtt

SEQ ID NO: 76 - DNA, 189 bp, Artificial Sequence
  Synthetic polynucleotide
ctatttagtaattttttcctcctgtcccaacttctgttctttcaagcaatgccctgccctaaggaagacactcccaattctgtttgggagcctgcgaaggcaaaatatgtctttagagatgtggtgcagataacctgtctggatgggtttgaagttgtggaggtaaagtaccaccttggcttctcccca

SEQ ID NO: 77 - DNA, 63 bp, Artificial Sequence
  Synthetic polynucleotide
agcttcgatatgactccacctgtgaacgtctntactatggagatgatgagaaatactttcgga

SEQ ID NO: 78 - DNA, 239 bp, Artificial Sequence
  Synthetic polynucleotide
tatttagaagctgctttgttccatgtaggtacaatatcctgacccaggaagatgctcagagtgtgtacgatgccagttattatgggggccagtgtgagacggtatacaatggggaatggagggagcttcgatatgactccacctgtgaacgtctctactatggagatgatgagaaatactttcggaaaccctacaactttctgaagtaccactttgaagtaagtctgaacagaggggct

SEQ ID NO: 79 - DNA, 63 bp, Artificial Sequence
  Synthetic polynucleotide
aggagagtaagacgggcagctacacccgcagnagttacctgccagctgagcaactggtcagag

SEQ ID NO: 80 - DNA, 161 bp, Artificial Sequence
  Synthetic polynucleotide
taaattttgcatctcaaaattgatgcatggatcttccctttctttaggagagtaagacgggcagctacacccgcagcagttacctgccagctgagcaactggtcagagtggacagattgctttccgtgccaggacaaaaaggtgagacacttacaaccggt

SEQ ID NO: 81 - DNA, 63 bp, Artificial Sequence
  Synthetic polynucleotide
aacttgttcttagcctgcccacatccacccangatccaaaacgggcattacattggaggacac

SEQ ID NO: 82 - DNA, 223 bp, Artificial Sequence
  Synthetic polynucleotide
tgtgtgggaacttgttcttagcctgcccacatccacccaagatccaaaacgggcattacattggaggacacgtatctctatatcttcctgggatgacaatcagctacatttgtgaccccggctacctgttagtgggaaagggcttcattttctgtacagaccagggaatctggagccaattggatcattattgcaaaggtgacttatttcttggtattcctta

SEQ ID NO: 83 - DNA, 63 bp, Artificial Sequence
  Synthetic polynucleotide
atgtggcctataagggcaatgcatacaatcantggtaggctctacctcggcaggtcctgttgt

SEQ ID NO: 84 - DNA, 197 bp, Artificial Sequence
  Synthetic polynucleotide
catacaatcattggtaggctctacctcggcaggtcctgttgtctgtgtggaggatgtagccgaagcggcaggagcagtagtagccgccaatgtagttgtggcagtagtggtcacaggacagctcctcgtcctccctctccttgcactcgtccacatctgtagggcaggtaaagcctctccatcaatacatgcatgat